(19) United States
(12) Patent Application Publication
(10) Pub. No.: US 2006/0115514 A1
(43) Pub. Date: Jun. 1, 2006

(54) Chelating and binding chemicals to a medical implant, medical device formed, and therapeutic applications

(76) Inventor: Stela Gengrinovitch, Merom Galil (IL)

Correspondence Address: Martin Moynihan;PRTSI, Inc., P.O. Box 16446, Arlington, VA, 22215, US

(21) Appl. No.: 11/284,832

(22) Filed: Nov. 23, 2005

Related U.S. Application Data
(60) Provisional application No. 60/630,560, filed on Nov. 26, 2004.

(51) Int. Cl. A61F 2/00; A61N 1/00

(52) U.S. Cl. 424/423; 607/2

(57) ABSTRACT

Chelating and binding chemicals to a medical implant, and therapeutic applications. Implantable metal chelated surface and chemical coated medical implant device—drug (or biological moiety) coated or drug eluting stent, prosthesis, or other, includes a medical implant component having metal surface (M) with chemical entity (X) bound via chelator (C) chelated to the metal surface in an (M)-(C)-(X) configuration. Chelator or/and chemical entity—drug (or biological moiety), linker bonded to a drug (or biological moiety), other, are bound at surface concentration greater than 100 picograms per cm². Manufacturing the implantable medical device. Medical implant system including medical implant component and delivery device for delivering and implanting medical implant component in a subject. Implanting the medical device. Preventing or/and treating medical conditions, such as restenosis or/and thrombosis, by implanting the medical device, wherein activity of bound chemical entity exhibits efficacy towards the medical condition.

### RELATED APPLICATIONS

This application claims the benefit of priority of U.S. Provisional Patent Application No. 60/630,560, filed on Nov. 26, 2004, the contents of which are incoporated herein by reference.

### FIELD AND BACKGROUND OF THE INVENTION

The present invention relates to medical devices in the form of medical implants or medical implant components to which are bound chemicals, manufacturing thereof, and therapeutic applications thereof, and more particularly, to a medical device featuring a medical implant or medical implant component having a metal surface to which is bound a chemical entity via a chelator chelated to the metal surface. The present invention further particularly relates to a method of manufacturing the medical implant device thereof, a medical implant system including the medical implant device thereof, a method of implanting the medical implant device thereof, a method of preventing or/and treating a medical condition of a subject using the medical implant device thereof, a chelate type of coordination compound including a drug or a biological moiety, and a medical device featuring a medical implant or medical implant component having a metal surface to which is chelated a chelator in a chelate configuration.

An exemplary medical implant or medical implant component having a metal surface which is particularly suitable for applying the present invention is a stent. Chemical entities which are suitable for applying the present invention are essentially any of a wide variety of different categories and types of chemical compounds, for example, a drug, a biological moiety, a linker or spacer capable of binding a drug or a biological moiety, and a linker or spacer to which a drug or a biological moiety is bound. In an exemplary preferred embodiment, the chelator is chelated to the metal surface of the medical implant or medical implant component, for example, a stent, in a form of a coating, whereupon the chemical entity (linker-drug or linker-biological moiety) bound to the metal surface via the chelator coating results in the formation of a drug (or a biological moiety) coated or drug (or a biological moiety) eluting medical implant device, for example, a drug (or a biological moiety) coated or drug (or a biological moiety) eluting stent, wherein activity of the bound chemical entity exhibits efficacy for preventing or/and treating a medical condition, disease, or ailment, such as restenosis, in general, in-stent restenosis, in particular, or/and thrombosis, in a human or animal subject.

The scope of implementation of the present invention is primarily focused toward application of a medical device in the form of a medical implant or medical implant component, for example, a stent, having a metal surface. In a non-limiting manner, the scope of implementation of the present invention clearly includes applications to various other medical devices in the form of a medical implant or medical implant component, which can have a metal surface, for example, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a prosthesis, a plate, a joint, a fin, a screw, a spike, a wire, a filament, a thread, an anchor, or a bone fixation element, among other exemplary medical devices.

Additionally, the scope of implementation of the present invention is directed toward application of the medical device for preventing or/and treating a medical condition, disease, or ailment, such as restenosis, in general, in-stent restenosis, in particular, or/and thrombosis, in a human or animal subject. In a non-limiting manner, the scope of implementation of the present invention clearly includes applications of the medical device for preventing or/and treating various other medical conditions, diseases, or ailments.

Basic principles and details relating to the chemistry, physics, and medicine, of the present invention, needed for properly understanding the present invention in an enabling manner are provided herein. Complete theoretical descriptions, details, explanations, examples, and applications of the relevant chemistry, physics, and medicine, and related subjects and phenomena, are readily available in standard references, including textbooks, articles, and the patent literature, in the fields of chemistry, physics, biology, and medicine, and sub-fields therein, for example, physical chemistry, inorganic chemistry, coordination chemistry, organic chemistry, organometallic chemistry, synthetic chemistry, biochemistry, biophysical chemistry, bio-inorganic chemistry, bio-organic chemistry, protein chemistry, pharmaceutical chemistry, pharmacology, medicinal chemistry, bio-medical science, materials science, cardiovascular medicine, pathology, medical implant technology, in general, and, stent technology, drug coated and drug eluting stent (DES) technologies, in particular.

Incompletely Solved Problem of Restenosis and In-Stent Restenosis (ISR)

As a direct result of the search for solutions to the well known problematic medical conditions of, and associated with, restenosis, in general, and in-stent restenosis (ISR) (also referred to as binary restenosis), in particular, which too often arise following treatment of intravascular ailments and diseases via interventional procedures of angioplasty and stent implantation, a plethora of prior art teachings, readily available in hardcopy and electronic forms of publication (textbooks, journals, governmental regulatory literature, and patent literature), has been, and continues to be, developed at a rapid and voluminous rate, in the areas of stent technology, in general, and drug coated and drug eluting stent (DES) technologies, in particular.

Separate from and prior to taking into account the already realized and potential results and benefits of the latest interventional procedures involving implantation of drug coated or drug eluting stents, as recently as last year, it was stated [Bhatia et al., 2003] “Much research has been done on many mechanical devices and drugs to prevent restenosis, providing the rationale for an enormous number of clinical trials, but none have been proven to be effective. Despite the use of multiple percutaneous revascularization techniques, including balloon angioplasty, repeated stenting, laser therapy, platelet inhibitors, heparin-coated stents and atheroablation, approximately half of the 30% of patients in whom restenosis occurs after coronary stenting, have recurrent restenosis”.

‘Causative’ Mechanism of Restenosis and In-Stent Restenosis

It has been well established and accepted that the main ‘causative’ mechanism of the phenomenon or condition of restenosis, in general, and in-stent restenosis, in particular, is not the progression of coronary artery disease, but rather the body's immune system response to the ‘injury’ of the interventional angioplasty or/and stent implantation.

Pathology and Biochemistry of Restenosis and In-Stent Restenosis

It has been stated that restenosis, in general, and in-stent restenosis, in particular, are “incompletely understood, biologically complex, and the Achilles' heel of endovascular treatment” [Smouse, H. Bob, 2003]. A recent description of the main events associated with the onset of restenosis, in general, and in-stent restenosis, in particular, following endovascular treatment is as follows [Smouse, H. Bob, 2003]. “Initiation of vascular wall trauma involves denuding of intima and stretching of media. This incites a cascade of molecular and cellular events, which lead to wound healing and restenosis. Wound healing occurs in three stages: (I) inflammatory phase (PLT and GT activation), (2) granulation phase (fibroblast and smooth muscle cell (SMC) migration to site of injury), and (3) remodeling phase (proteoglycan and collagen synthesis in extra cellular matrix). A cascade of the events of platelet deposition, leukocyte recruitment, VSMC migration/proliferation, and matrix deposition, leading to wound healing also leads to in-stent restenosis”.

Another recent and more detailed description of the onset of in-stent restenosis is as follows [Bhatia et al. (2003)]. “The initial events immediately after stent placement result in de-endothelialization and the deposition of a layer of platelets and fibrin at the injured site in the coronary artery. Activated platelets express adhesion molecules such as P-selectin and glycoprotein (GP) Ib [alpha], which attach to circulating leukocytes via platelet receptors such as P-selectin glycoprotein ligand and begin a process of rolling along the injured surface. Under the influence of cytokines, leukocytes bind tightly to the leukocyte integrin (i.e., Mac-1) class of adhesion molecules via direct attachment to platelet receptors such as GP Ib[alpha] and through cross-linking with fibrinogen to the GP IIb/IIIa receptor. The migration of leukocytes across the platelet-fibrin layer and into the tissue is driven by chemical gradients of cytokines released from smooth muscle cells (SMCs) and resident leukocytes. Growth factors are released from platelets, leukocytes, and SMCs, which influence the proliferation and migration of SMCs from the media into the neointima. The resultant neointima consists of SMCs, extracellular matrix (ECM), and macrophages recruited over several weeks. Over even longer periods of time, there is a shift to fewer cellular elements with greater production of extracellular matrix. In addition, there is eventual re-endothelialization of at least part of the injured vessel surface”.

Accordingly, migration and proliferation of vascular smooth muscle cells as well as reorganization of the extracellular matrix are major events in the formation of intimal lesions during atherosclerosis and restenosis following balloon angioplasty [Ross R., 1997; Coats, W. D., et al., 1997; and Batchelor, W. B., et al., 1998].

The extracellular matrix (ECM) consists mainly of fibrous proteins and structured sugars. ECM fibrous proteins are of two functional types: structural, such as collagen and elastin, and adhesive, such as fibronectin and laminine. ECM structured sugars are mainly polysaccharide glycosaminoglycans, such as hyaluronic acid, chlondroitin sulfate, dermatan sulfate, heparan sulfate, heparin, and keratan sulfate [Hay, E. D., 1981; McDonald, J. A., 1988; Piez, K. A., et al., 1984]. ECM remodeling involves a wide variety of different types of enzymes that control the process. Exemplary ECM remodeling types of enzymes are proteases, such as matrix metalloproteinases (MMPs), serine-type peptidases, threonine-type peptidases, aspartic-type peptidases, and cystein-type peptidases. Other enzymes, such as lipid or sugar degrading enzymes, also can play a role in extracellular matrix remodeling, among them enzymes that degrade structured sugars of the matrix, such as heparinase and hyaloronidase.

Major drivers that induce vascular remodeling and matrix metalloproteinase (MMP) expression and activation are: injury, inflammation, and oxidative stress. All these factors play an important role in restenosis, in general, and in-stent restenosis, in particular. Many different types of matrix metalloproteinases (MMPs) are involved in vascular remodeling and atherogenesis. MMPs that were shown to be involved in vascular remodeling are: MMP-1, MMP-2, MMP-3, MMP-7, MMP-9, MMP-12, MMP-13, and MMP-14 [Zorina, S., et al., 2002]. All of these MMPs are produced by human macrophage cells. MMP-1, 2, 3, 9, and 14, are produced by SMCs both in-vitro and in animal studies. There are animal studies that show differential expression of MMPs after stent implantation and balloon injury.

There is extensive evidence suggesting that SMCs produce plasminogen activators and MMPs in response to vessel wall injury [Clowes, A. W., 1990; Jackson., C. L., 1993; Zempo, N., et al., 1994; Reidy, M. A., et al., 1996; Shofuda, K., et al., 1998]. For example, arterial injury causes expression and activation of MMP-2 and MMP-9, and this is associated with increased migration and proliferation of SMCs [Zempo, N., et al., 1994; Bendeck, M. P., 1994]. Several other MMPs are also expressed in human atherosclerotic lesions, including stromelysin (MMP-3), interstitial collagenase (MMP-1) and type IV collagenases (MMP-2 and MMP-9) [Henney, A., et al., 1991; Galis, Z. S., et al., 1994; Brown, D. L., et al., 1995].

Intimal hyperplasia is the principal mechanism of restenosis, in general, and in-stent restenosis, in particular. Studies of MMP expression following stent implantation show over-expression of MMP-9 and activation of MMP-2 in animal models [Feldman, L. J., et al., 2001]. Neointima formation in organ cultured human Saphenous vein grafts is inhibited by simvastatin (investigational new drug (IND)), and is associated with MMP-9 reduced activity and inhibition of SMC proliferation and migration [Porter, K. E., et al., 2002]. FUT-175, a serine protease inhibitor, also inhibits neointimal formation after balloon injury in rats [Sawada, M., et al., 1999].

Many MMP substrates and inhibitors have been identified [Whittaker, M., et al., 1999]. Most of MMP substrates are native proteins of the ECM in which the specific peptide sequence that is being cleaved was identified [Netzel-Arnett, S., et al., JBC, 1991; Netzel-Arnett, S., Anal. Biochem., 1991; Niedzwiecki, L., et al., 1992].

Thrombosis Via Restenosis

Thrombosis, or blood clotting, begins with activation of factors in the blood and adhesion of platelets to vascular tissue, usually around the area of a valve. A cascade of reactions leads to the formation of a fibrin mesh to reinforce the blood clot as platelets. Such activation and reactions may take place simultaneously with, or subsequently to, onset or/and progress of restenosis, in general, and in-stent restenosis, in particular, or/and the pathological and biochemical processes associated with restenosis. Current methods of preventing or inhibiting occurrence of thrombosis resulting from processes associated with restenosis typically involves systemic administration of anti-coagulant and anti-clotting medications for at least several weeks immediately following stent implantation. Nevertheless, preventing or inhibiting thrombosis in all cases is not guaranteed. Finding ways of preventing or treating restenosis, in general, and in-stent restenosis, in particular, may lead to preventing or inhibiting occurrence of thrombosis caused or induced by restenosis. Clearly, preventing or inhibiting occurrence of thrombosis is highly desirable, in order to prevent or inhibit any number of potentially problematic side effects, phenomena, or/and conditions, such as an embolism, associated with or/and caused by thrombosis.

Preventing or/and Treating Restenosis Via Systemic and Brachytherapy Techniques

A currently well known and used therapeutic technique for attempting to prevent or/and treat restenosis, in general, and in-stent restenosis, in particular, is based on systemic pharmacological therapy combined with or immediately following stent implantation. However, as stated by Bhatia et al. (2003), “Experience with systemically administered drugs, such as antiplatelet agents, anticoagulants, calcium-channel blockers, angiotensin-converting-enzyme inhibitors, cholesterol-lowering agents, and antioxidants, has proven almost universally negative”. “These agents were previously tested in animal models and found to be beneficial.” However, “. . . therapeutic success of anti-restenotic therapies has not been achieved in human beings”. Moreover, “Similarly, the results with oral administration of an anti-proliferative agent, sirolimus, have failed to show any benefit and in fact there was a higher incidence of adverse events in the recipients of such a therapy”.

Another currently well known and used therapeutic technique for attempting to prevent or/and treat restenosis, in general, and in-stent restenosis, in particular, is based on the use of intracoronary radiation (brachytherapy). The localized irradiation of a blood vessel from within the vessel, as part of, or immediately following, angioplasty or/and stent implantation, has been found to be effective in reducing the incidence of restenosis. To date, such radiation has been locally delivered to the blood vessel via a number of different medical devices and techniques, including, for example, by guide wire, balloon, temporarily implantable wire, or permanently implantable stent. The medical device is either partially or wholly formed of radioactive material, or alternatively, is coated with a radioactive substance. Material giving off high levels of radiation may be briefly introduced into the body and then removed. Alternatively, material giving off a relatively lower level of radiation and with an appropriately short half-life may be introduced temporarily, or alternatively, left in place, for example, as with a radioactive stent or a radioactive coated stent.

As stated by Bhatia et al. (2003), “The recent introduction of intracoronary radiation has emerged as a promising modality to attenuate the intimal hyperplastic reaction. Despite the lack of benefit for preventing restenosis in de-novo lesions, brachytherapy was shown to be effective in reducing recurrent restenosis. However, larger studies and long-term follow-up showed alarming long-term sequelae such as edge restenosis and late thrombosis, raising some concerns about the potential toxicity of a cytotoxic approach”. Other unfavorable side effects, such as inhibition of healing around the stent and increased risk of cancer, lead to the conclusion that brachytherapy is currently not the best treatment for preventing or treating restenosis. Studies [Cardiac. Consult., 2001] have been performed for attempting to gain an understanding of long-term effects of radiation and the use of stent-based brachytherapy, beta radiation, and pharmachologic agents along with brachytherapy, in order to improve long-term outcomes.

Preventing or Minimizing Restenosis Via Bare Stent Design and Construction

“There is increasing evidence that stent design influences angiographic restenosis and clinical outcomes” [McClean, D. R., et al., 2002]. As stated therein, “Thus, it seems that the specific metallic composition of a stent has two ways to influence restenosis: the limits metallurgy imposes on mechanical properties affect the universe of stent geometries possible which impact on implantation injury, and the biocompatibility of the metal may affect long-term stent healing. Stent geometry, dimensions such as length and thickness, and stent surface properties (for example, microscopic roughness) appear to highly influence both thrombosis and restenosis rates. Prior to combination antiplatelet therapy, a higher metal surface area was thought to facilitate thrombus formation. In a bid to reduce the percentage metal surface area and also to improve access to side branches, stents with larger or open cells were designed”.

Furthermore, as described therein, “Studies have shown that stent geometry designed to optimize expansion and lower recoil is a prerequisite for favorable clinical outcomes. Evidence from animal models show that stent geometry and thickness can affect experimental vascular injury and neointimal proliferation. Strut thickness appears to be an important risk factor for restenosis, but changing one parameter, such as strut thickness, requires altering other design characteristics, thus altering the overall stent design. Chronic inflammation might also result from electrochemical forces on the surface of stent struts, which may also increase stent interactions with circulating proteins. Taken as a whole, these data tell us that a variety of design parameters, including cell geometry, strut thickness, acute recoil, and surface characteristics, have an important effect on clinical outcomes. Future stent designs should combine the best features of conventional stent design with special modifications to facilitate multi-agent drug elution for a variety of applications”.

Preventing or/and Treating Restenosis Via a Drug Coated/Drug Eluting Stent (DES)

As a consequence of currently known systemic pharmacological or brachytherapy techniques, as well as techniques for customizing or/and optimizing physical parameters of bare stent design and construction, failing to provide a sufficiently effective, consistent, robust, and safe, solution to restenosis, in general, and in-stent restenosis, in particular, there has been ongoing research, development, testing, and use, of alternative techniques for preventing or/and treating restenosis.

Currently, the newest development in the ongoing battle to prevent, or at least reduce, restenosis, in general, and in-stent restenosis, in particular, is what is commonly, and usually synonymously, known as a drug coated stent or drug eluting stent (DES), which is also referred to as a drug medicated stent. Although definable in slightly different ways, in general, a drug coated or drug eluting stent is a medical device in the form of a medical implant or medical implant component being a stent which has medication (at least one bioactive or pharmacological agent in the form of a drug, where, for brevity and generality, such medication is usually referred to as a drug) coated on it in order to prevent or/and inhibit the onset or/and progress of restenosis, in general, and in-stent restenosis, in particular, via interfering with one or more of the several mechanisms and processes (for example, inflammation, granulation, ECM remodeling, as described hereinabove) associated with the onset or/and progress of restenosis, in general, and in-stent restenosis, in particular.

Structural Components. Functions, and Operation of Drug Coated/Drug Eluting Stents

Currently, most drug coated or drug eluting stents feature three main structural components, and, associated functions and aspects of each: (1) the bare stent that provides host to, and carries, the medicated coating, and the type, properties, characteristics, and behavior, of the bare stent; (2) the coating that coats the bare stent and provides host to, and carries, the medication, and the type, properties, characteristics, and behavior, of the coating with respect to its physicochemical relationship and interaction with the bare stent; and (3) the medication that is carried by the coating, and the type, properties, characteristics, and behavior, of the medication with respect to its physicochemical relationship and interaction with the coating, and with the immediately surrounding media (blood vessel solids and fluids). There are teachings about drug coated or drug eluting stents which have no separate coating upon the bare stent, whereby the medication is directly coated, adhered, or adsorbed, typically, via mechanisms involving hydrophobic interaction or/and physical adsorption, onto the surface of the bare stent. The overall functionality of a drug coated or drug eluting stent, with respect to efficacy and pharmacokinetics of the drug is directly dependent upon the type, properties, characteristics, and behavior, of the mechanism(s) by which the medication (at least one drug), via the coating (if present), is eluted, delivered to, and interacts with, the immediately surrounding media (blood vessel solids and fluids).

For those types of drug coated or drug eluting stents which include a separate coating upon the bare stent, currently, the two main types of mechanisms by which a drug, via the coating, is eluted and delivered to the immediately surrounding media are matrixing and conjugation. Matrixing is primarily based on ‘physical’ mixing of a drug and a polymer coating (either bioerodable (biodegradable) or non-bioerodable (non-biodegradable)), whereby the drug is physically dispersed and embedded throughout the polymer matrix, and is controllably released from the polymer matrix and transported to the surrounding media by diffusion. Conjugation is primarily based on ‘chemical’ attachment, via covalent bonding, of a drug to a polymer coating, whereby the drug is controllably released from the polymer matrix by bioerosion (biodegradation) of the bioerodable (biodegradable) polymer via enzymatically based surface erosion.

As described in the literature [Bhatia et al., 2003], “A drug eluting stent is a device that releases single or multiple bioactive agents into the bloodstream that can deposit in or around tissues adjacent to the stent. With such drug coated stents, there is site-specific drug delivery, which reduces systemic toxicity and thus is an attractive therapeutic method to achieve an effective local concentration of a drug for a designed period. The safety and efficacy of such an approach critically depends on the delicate combination of drug, polymer, and the kinetics of release. The drug can be simply linked to the stent surface, embedded and released from within polymer materials, or surrounded by and released through a carrier. The carrier can coat (strut-adherent) or span (strut-spanning) the stent struts”.

Additionally, as described in the literature [Frake, P., et al., 2004], drug eluting stents are “ . . . designed to locally deliver drugs in order to inhibit neointimal hyperplasia without the serious effects of radiation or systemic drug administration. These coated, or drug eluting, stents used various drugs encapsulated in different polymeric and non-polymeric formulations. Drug eluting stents were found to greatly reduce restenosis and, in the short term, have been found to maintain arterial patency better than surgical interventions such as bypass grafts. This, combined with a lower incidence of side effects and a lower cost, makes drug eluting stents a viable option for treatment of coronary artery disease (CAD). Though their track record is, at this point, quite impressive, the long term efficacy and implications of drug eluting stents remains to be seen”.

Types of Bare Stents Usable in Drug Coated/Drug Eluting Stents

Prior art teaches about a vast variety of many different types of compositions of bare metal stents having a correspondingly vast variety of different physicochemical and mechanical properties, characteristics, and behavior, along with a plethora of many different geometrical configurations, shapes, forms, and dimensions, of the overall stent frame, skeleton, or scaffold, and of the cells thereof, which are usable in drug coated/drug eluting stents. The bare stents of drug coated/drug eluting stents are ordinarily composed of materials which are made of stainless steel, or/and shape memory alloy (SMA) materials or/and alloys thereof, or/and combinations thereof. Selected examples of known and used shape memory alloy materials and alloys are: Ni—Ti (Nitinol™), Co—Mo—Cr, Be—Cu, Co—Cr (Elgiloy™), Co—Cr, Co—W, Ni—Ti—V, Pt—Ir, Cu—Zn—Al, Pt—W, Co—Cr—Ni, Ni—Co—Cr—Mo, where any of these alloy materials may be metal coated or plated, for example, with a silver or/and gold metal coating or plating.

Types of Polymers Usable in Polymer Coating Based Drug Coated/Drug Eluting Stents

As described in the literature [Frake, P., 2004], “The basic mechanism of drug delivery from a polymeric scaffold involves encapsulating a drug in a polymer that either allows the drug to diffuse outward from it or that undergoes degradation in order to release the drug directly. Polymers can be subdivided into bioerodable and nonbioerodable categories. The bioerodable polymers can be further subdivided into either bulk or surface erosion. Generally, for long term applications, such as in stents, a nonerodable polymer (rather than an erodable polymer) is used. This is because the fragments that break off from the polymer coating, particularly in polymers that undergo bulk erosion, tend to be phagocytosed by macrophages and other lymphocytes. Phagocytosis of polymer fragments can trigger macrophage activation, which release inflammatory cytokines, leading to increased lymphocyte infiltration of the site leading to inflammation. Numerous polymer systems that seemed promising in vitro have subsequently been abandoned after in vivo studies demonstrated inflammatory responses to them. Generally nonbioerodable, or biostable, polymers are used in more permanent biological applications, like stents, because of the potential for the occurrence of bioincompatibilty when using erodable polymers, and due to the more gradual release of drug that nonbioerodable polymers provide”.

Types of Medications (Drugs) Usable in Drug Coated/Drug Eluting Stents

Drugs that have been clinically proven to be useful for preventing or/and inhibiting restenosis, in general, and in-stent restenosis, in particular, via drug coated/drug eluting stents, fall into four major categories: (1) anti-neoplastics (anti-inflammatories), (2) immunosupressives (anti-proliferatives), (3) migration inhibitors (ECM modulators), and (4) enhanced healing (re-endothelialization) factors. Drugs in the category of anti-platelets (anti-coagulants) are also usable in drug coated/drug eluting stents, for preventing or/and inhibiting onset or/and progress of thrombosis which may occur along with, or as a result of, restenosis.

Examples of Polymer Coating Based Drug Coated Stents

An example of a polymer coating based drug coated (non-eluting) stent is the FDA approved HEPACOAT™ drug coated stent (Cordis/Johnson & Johnson, U.S. Pat. No. 5,336,518), which is coated with a polymer coating to which is covalently bonded, either directly or via a spacer, to the anticoagulant drug heparin. This particular polymer coating based drug coated stent functions by the heparin remaining chemically secured (covalently bonded) to, without eluting from, the polymer coating, as blood vessel fluids contact it by flowing along the outer surface of the polymer coating.

As disclosed in U.S. Pat. No. 5,336,518, the metal surface of a medical device, in this case, a stent, is rendered biocompatible by coating the metal surface with a layer of heptafluorobutylmethacrylate (HFBMA) monomer to form a polymer coating on the surface, treating the polymer coating with water vapor plasma (via radiofrequency (RF) plasma deposition) to provide reactive (carboxy and hydroxy) groups thereon, and covalently bonding a biologically active agent (a drug, in this case, heparin) to the polymer coating. The HFBMA polymer coating is exposed to an aqueous heparin solution having a heparin concentration of between about 4.0 mg/ml and about 8.0 mg/ml for a period of between about 30 and about 90 minutes. Alternatively, a spacer group can be bonded to the activated HFBMA coating and the biologically active agent (heparin) can then be bonded to the spacer group. Apparently, the HFBMA coatings are durable even under severe crimping and expansion conditions, such as occurring with stent implantation. The thus formed biocompatible metallic based medical device (stent), when implanted within a blood vessel, is stated as preventing substantial thrombus from occurring on its surface while not significantly interfering with endothelialization of the metal surface, and also as preventing promotion of smooth muscle cell proliferation and therefore restenosis.

The motivation for the invention disclosed in U.S. Pat. No. 5,336,518, was to provide a medical device, such as a stent, having a biocompatible metal surface with an anti-thrombogenic agent, such as heparin, chemically secured (via chemical bonding) thereto, whereby the anti-thrombogenic agent would withstand flexure and interaction with fluids, thereby remain secured for its entire active lifetime and only minimally leach away in a wet environment, such as that encountered in a blood vessel. Accordingly, the HEPACOAT drug coated stent properly functions by the heparin remaining chemically secured to, without eluting from, the HFBMA polymer coating.

A review of clinical studies reveals that in some cases the HEPACOAT drug coated stent meaningfully prevented or inhibited occurrence of thrombosis resulting from processes associated with restenosis, whereas in other cases the occurrence of stent thrombosis was the same as that when using a non-coated bare stent, but in no case was it definitively proven that the HEPACOAT drug coated stent is suitable for preventing or treating restenosis, in general, and in-stent restenosis, in particular.

Another example of a polymer coating based drug coated (non-eluting) stent is disclosed in U.S. Pat. Pub. No. 2003/0229393 A1, by Kutryk, M. J. B., et al. As disclosed therein, the medical device, for example, in the form of a stent, is coated with a polymeric biocompatible matrix coating to which is covalently bonded, either directly or via a spacer, a protein layer. The protein layer components remaining chemically secured (covalently bonded) to, without eluting from, the polymeric biocompatible matrix coating, as blood vessel fluids contact it by flowing along the outer surface of the polymer coating. The polymeric biocompatible matrix coating may be a synthetic material, for example, a polyurethane, a segmented polyurethane-urea/heparin, a poly-L-lactic acid, cellulose ester, polyethylene glycol, polyvinyl acetate, dextran, or gelatin, or, alternatively, a naturally occurring material, for example, collagen, elastin, laminin, fibronectin, vitronectin, heparin, fibrin, cellulose, or amorphous carbon, or, alternatively, a fullerene ranging from about C₂₀ to about C₁₅₀ in the number of carbon atoms. The protein layer is preferably composed of two kinds of proteins: (1) one or more types of antibodies which recognize, bind to, or/and interact with, a progenitor cell surface antigen to immobilize and promote adherence of endothelial cells at the surface of the stent, and (2) one or more growth factors which stimulate endothelial cell growth and differentiation. A main objective is that upon implantation of the stent, the cells that adhere to the surface of the stent will transform into a mature, confluent, functional layer of endothelium on the luminal surface of the stent, whereby the presence of the confluent layer of endothelial cells on the stent will reduce the occurrence of intimal hyperplasia, restenosis, or/and thrombosis, at the site of implantation.

Examples of Polymer Coating Based Drug Eluting Stents

Two examples of FDA approved polymer coating based drug eluting stents, wherein each is based on a stent platform upon which a cytotoxic drug is matrixed and embedded throughout a non-erodable polymer coating on the surface of the stent, and is controllably released (eluted) from the polymer matrix by diffusion into the immediately surrounding media (blood vessel solids and fluids), are the CYPHER™ sirolimus-eluting stent (Cordis/Johnson & Johnson, U.S. Pat. Nos. 6,585,764; 6,273,913), and the TAXUS™ paclitaxel-eluting stent system (Boston Scientific, U.S. Pat. Nos. 6,344,028; 6,197,051; 6,179,817). The CYPHER and TAXUS drug eluting stents carry extremely low doses of drugs (typically, on the order of μgs drug per mm² stent surface area) that temporarily inactivate cells with the artery wall, keeping them from multiplying and overgrowing the stent. CYPHER uses the anti-organ-rejection (immunosuppressive) drug sirolimus (rapamycin); TAXUS uses the anti-cancer (chemotherapeutic) drug paclitaxel. Both the CYPHER and TAXUS drug eluting stents have shown significant reduction of restenosis in clinical trials and in the field as well.

The CYPHER stent is composed of three layers of polymers over a frame made of laser cut 316L stainless steel. This metal stent is electropolished and coated in a primer layer of Parylene C. A mixture of polyethylene-co-vinyl acetate (PEVA) and poly n-butyl methacrylate (PBMA) in then dissolved in THF, which is a solvent suitable for dissolving organic molecules. This copolymer has a ratio of PBMA to PEVA of about 67% PEVA, 33% PBMA. Sirolimus is then dissolved in the THF/polymer mixture and the mixture is applied to the Parylene C coated stent. Another mixture of PEVA and PBMA, without sirolimus, is dissolved in THF and applied to the stent by spraying with a fine nozzle. This outer coating prevents the so-called ‘burst effect’ which results when drug on the surface of the polymer is rapidly released following immersion in water or another solvent. A small amount of sirolimus migrates to the final layer during this step because it dissolves in the THF and precipitates in the PEVA/PBMA outer layer, causing a small but noticeable burst effect. The entire three layered coating is applied to both the luminal and abluminal sides of the stainless steel stent. Finally, the stent is placed on a delivery catheter, sterilized, and packaged.

Sirolimus is released from the CYPHER stent via the PEVA/PBMA polymeric layers into the surrounding area by diffusion. This mechanism can be described by Fick's law of diffusion, and is dependent upon concentration of drug both inside and outside the polymer matrix. The greater the difference between drug concentration inside and outside the polymer matrix, the faster the release of drug will occur. As previously stated, the outer layer of PEVA/PBMA minimizes the burst effect following stent implantation. The outer PEVA/PBMA layer also slows the rate of sirolimus diffusion allowing the drug to be released gradually over a longer period. The concentration of drug decreases with first order elimination kinetics. Approximately 50 percent of the total drug is eliminated within the first 10 days of implantation. The drug is 90 percent removed from the stent by about 60 days, and is completely removed by about 90 days following implantation. The peak drug concentration occurs about 4 hours after implantation. This release profile provides just enough drug release immediately after stent implantation to prevent neointimal hyperplasia, without any of the side effects of systemic administration.

Regarding the CYPHER drug eluting stent, as described in the literature [Bhatia et al., 2003], “The potential usefulness of immunosuppressive agents in the treatment of restenosis arises from parallels between tumor cell growth and the benign tissue proliferation, which characterizes intimal hyperplasia. Sirolimus is a natural macrocyclic lactone with potent immunosuppressive and antimitotic action, which was approved in 1999 as an anti-rejection drug in renal transplant recipients. The cellular action of rapamycin (sirolimus), a natural fermentation product produced by Streptomyces hygroscopicus, is mediated by binding to the FK506 binding protein. By inhibiting a kinase known as the target of rapamycin, it restricts the proliferation of smooth-muscle cells by blocking cell-cycle progression at the G1/S transition. The finding that rapamycin possesses both anti-proliferative and anti-migratory activity suggests that it could contribute to the control of arterial re-narrowing after percutaneous intervention”.

The TAXUS stent is constructed out of 316L stainless steel and is coated with the translute polymer [poly(styrene-b-isobutylene-b-styrene)]. This polymer functions similarly to the PEVA/PBMA copolymer used in the CYPHER stent. This polymer is also notable for its excellent vascular compatibility, which is extremely important in a system designed for long-term implementation. The pharmacokinetics of the paclitaxel release are slightly different from the CYPHER stent: burst release in the first 48 hours, slow release over the next 10 days, and no further release after 30 days.

Regarding the TAXUS drug eluting stent, paclitaxel, in the anti-neoplastic family of compounds, also inhibits the cell cycle, but works via a different mechanism than sirolimus. Paclitaxel binds to microtubules in dividing cells and causes them to assemble, thereby preventing mitosis. As further described in the literature [Bhatia et al., 2003], “The taxanes (for example, paclitaxel) are potent anti-proliferative agents used in cancer chemotherapy. Paclitaxel promotes polymerisation of the alpha and beta subunits of tubulin by reversibly and specifically binding the beta subunit of tubulin, and thus stabilizes microtubules. A stent coated with paclitaxel is also safe and effective for decreasing neointimal proliferation within the stented segment and reducing the incidence of clinically significant in-stent or edge restenosis”.

Two examples of a hydrophobic type of a polymer coating based drug eluting stent are taught about in U.S. Pat. No. 6,716,445, to Won, et al., and in U.S. Pat. No. 6,702,850, to Byun, et al. The drug eluting stent of U.S. Pat. No. 6,716,445 features the use of a hydrogel as a hydrophobic macromer entrapping the drug. The drug eluting stent of U.S. Pat. No. 6,702,850 features the use of a hydrophobic polymer that is covered with linked heparin as the outer layer of the structure. This is aimed at preventing thrombosis in addition to the anti-restenosis properties of the drug and the stent itself.

Limitations of Polymer Coating Based Drug Coated/Drug Eluting Stents

In general, polymer coating based drug coated or drug eluting stents are inherently limited due to the mere presence of the polymer coating as an integral component of the drug coated or drug eluting stent. The polymer coating serves as a temporally (time) dependent intermediary between the bare stent and the medication (drug), as well as the immediately surrounding media (blood vessel solids and fluids). Safe and efficacious construction and function of a polymer coating based drug coated or drug eluting stent are directly related to the physicochemical type, properties, characteristics, and behavior, of the polymer coating with respect to its physicochemical relationship and interaction with the bare stent, with the medication, and with the immediately surrounding media, as a function of time. Thus, there exist a relatively large number of time dependent parameters and factors directly associated with the polymer coating which need to be fully analyzed, tested, and understood, in order to provide a safe and effective design, construction, implantation, and employment, of a polymer coating based drug coated or drug eluting stent.

A particularly significant limitation associated with the polymer coating of a polymer coating based drug coated or drug eluting stent relates to safety of a subject following stent implantation. As previously stated above, generally non-bioerodable, or biostable, polymers are used in stents, because of the potential for the occurrence of bioincompatibilty when using erodable polymers, and due to the more gradual release of drug that nonbioerodable polymers provide. Ultimately, however, after a sufficient amount of time in the body of a subject, even so-called nonbioerodable or biostable polymers used in polymer coating based drug coated or drug eluting stents erode, degrade, or/and decompose, to some extent over time as long as they remain in the body, for example, due to oxidative decomposition of polymers by human macrophages or active enzymatic reactions. The polymer coating or/and erosion, degradation, or/and decomposition, products thereof, may potentially lead to any number of undesirable side effects and phenomena, such as chronic, low-grade inflammation, poor wound healing response with incomplete endothelialization, or/and intra-hemorrhage, which themselves have been proven to lead to the problematic conditions of in-stent restenosis or/and thrombosis.

Another notable safety limitation associated with the polymer coating (erodable or non-erodable type) of a polymer coating based drug coated or drug eluting stent is the always existing possibility that the polymer coating may contain potentially unsafe levels of impurities or/and contaminants, which would be introduced into the body via the polymer coating or/and further dispersed throughout the body via erosion, degradation, or/and decomposition, products thereof.

A potential functional limitation associated with the polymer coating (erodable or non-erodable type) of a polymer coating based drug coated or drug eluting stent is the always existing possibility that the polymer coating or/and erosion, degradation, or/and decomposition, products thereof, may physically or/and chemically modify or damage the matrixed or conjugated drug, leading to reduced efficacy, along with the possibility that unknown undesirable side effects, phenomena, or/and conditions, may arise.

To date, an ideally structured and functioning polymer coating of a polymer coating based drug coated or drug eluting stent, which itself, and its erosion, degradation, or/and decomposition, products, are sufficiently harmless inside the body for long periods of time, so as not to lead to, or potentially lead to, any number of undesirable side effects, phenomena, or/and conditions, which subsequently lead to in-stent restenosis or/and thrombosis, has not yet been identified.

Polymer-Free Based Drug Coated/Drug Eluting Stents and Limitations Thereof

Unless the drug itself is a polymer, a polymer-free based drug coated or drug eluting stent can be made by dipping the bare metal stent into a solution of a drug, or by applying a solution of a drug onto the luminal or/and abluminal surface of the bare stent, such that the drug itself becomes directly coated, adhered, or adsorbed, typically, via mechanisms involving hydrophobic interaction or/and physical adsorption, onto the surface of the stent. The overall functionality of such a polymer-free based drug coated or drug eluting stent, with respect to efficacy and pharmacokinetics of the drug is directly dependent upon the type, properties, characteristics, and behavior, of the drug coating with respect to its physicochemical relationship and interaction with the bare stent, and with respect to the type, properties, characteristics, and behavior, of the mechanism(s) by which the medication (at least one drug) interacts with the immediately surrounding media (blood vessel solids and fluids), and if applicable, is eluted and delivered thereto.

Recently [Gershlick, A., et al., 2004], a polymer-free based ‘paclitaxel’ drug eluting stent (V-Flex Plus coronary stent, Cook Inc.) was evaluated in Europe for safety and efficacy with respect to inhibition of in-stent restenosis. Escalating doses of paclitaxel (0.2, 0.7, 1.4, and 2.7 μg/mm² stent surface area) were directly applied to the stent, which was then implanted in the immediate vicinity of de novo lesions. Application of the paclitaxel was done by dipping or immersing the abluminal surface of the stent in an ethanolic solution of paclitaxel followed by evaporating the solvent, thereby leaving a fine residue of the paclitaxel drug that adheres to the metal surface. Compared to treatment using a bare stent alone, a dose density of 2.7 μg/mm² of the paclitaxel eluting stent reduced angiographic indicators of in-stent restenosis, without short- or medium-term side effects.

A potentially significant limitation of such a polymer-free based drug coated or drug eluting stent is that the drug is ‘physically’ coated, adhered, or adsorbed, onto the surface of the stent, and is not ‘chemically’ adsorbed or attached, via covalent bonding, to the surface of the stent. Compared to a chemisorbed layer or coating of a chemical, such as a drug, on a metal surface, as a function of time, a physisorbed layer or coating of a drug on a metal surface is usually more vulnerable, and has properties, characteristics, and behavior, which are more variable, to changes in environmental conditions and external effects, and therefore, is less likely to function in a highly predictable, consistent, and efficacious manner.

Ligands, Chelators, Coordination Compounds, Complexes, and Coordination Chemistry

Herein, consistent with prior art theories, principles, practices, and applications, well known and used in the field of chemistry, and in various sub-fields and related fields thereof, the term ‘ligand’ refers to a chemical specie (molecule, compound) having at least one coordinating group which is able to complex (coordinate) with a metal ion. A ligand has a negative charge (anionic), a zero charge (neutral), or a positive charge (cationic). The term ligand is synonymously known, and equivalently referred to, as a ‘complexing agent’. Herein, for purposes of preserving clarity and consistency in meaning, understanding, and usage, the term ‘ligand’, instead of the term ‘complexing agent’, is used throughout, unless otherwise clearly indicated.

Based on the definition of ligand, a ‘chelator’ specifically refers to a ligand (complexing agent) having more than one coordinating group in its structure. A ligand with two coordinating groups in its structure is commonly called a bidentate (two-toothed) or bifunctional ligand. A ligand with three, four, five, six, seven, or eight, coordinating groups is commonly called a terdentate (three-toothed) or trifunctional ligand, a quadridentate (four-toothed) or quadrifunctional ligand, a pentidentate (five-toothed) or pentafunctional ligand, a hexidentate (six-toothed) or hexafunctional ligand, a heptidentate (seven-toothed) or heptafunctional ligand, an octidentate (eight-toothed) or octafunctional ligand, respectively. These and higher number coordinating group multi-dentate or multifunctional ligands are generally referred to as chelators, chelating groups, or as chelating agents.

In prior art teachings, a chelator, chelating group, or chelating agent, is also generally known, and equivalently referred to, as complexing agent, with an explicit or implicit understanding that the particular complexing agent referred to has more than one coordinating group in its structure. Herein, similar to usage of the term ‘ligand’, as explained immediately above, for purposes of preserving clarity and consistency in meaning, understanding, and usage, the terms ‘chelator’, ‘chelating group’, or ‘chelating agent’, instead of the term ‘complexing agent’, are used throughout, unless otherwise clearly indicated.

A few selected examples of multi-dentate or multifunctional ligands (complexing agents), or chelators, commonly known and used in a wide variety of fields for a wide variety of different applications, and which are suitable for implementation of the present invention, are ethylenediamine (en), having two coordinating groups; propylenediamine (pn), having two coordinating groups; diethylenetriamine (dien), having three coordinating groups; triethylenetetraamine (trien), having four coordinating groups; ethylenediaminetetraaceto (EDTA), having six coordinating groups; oxalic acid, having two coordinating groups; and 8-hydroxyquinolate, having four coordinating groups. Multi-dentate or multifunctional ligands (complexing agents), or chelators, can be more complex molecules, such as peptides, polypeptides, and proteins.

Chemical reaction of a multi-dentate or multifunctional ligand (complexing agent), or chelator, with a metal ion produces a particular type of chemical complex commonly known by a variety of synonymous names and terms, in particular, as a metal complex, as a metal ion complex, as a coordination compound, as a coordination complex, as a chelate complex, as a chelate ring, or, for brevity, as a chelate, since the structure of the chemical complex so produced is typically in the form of a chelate (claw-like) ring. The type of chemical bond formed between the central metal ion (or atom) and each coordinating group of the chelator in the coordination compound or chelate is a ‘coordinate covalent bond’, which is also equivalently known as a polar covalent bond, since, in contrast to forming a ‘regular’ covalent bond, in forming a ‘coordinate’ covalent bond both electrons of the bond have been contributed by the coordinating group and the metal ion merely accepts a share in the electron pair. For brevity, and for most theoretical and practical purposes, such a coordinate covalent bond, or polar covalent bond, in a coordination compound or chelate, is commonly also referred to as a covalent bond.

A chelator involved in the formation of a coordination compound or chelate has a negative charge (anionic), a zero charge (neutral), or a positive charge (cationic), with a negative charge being most common, a zero charge less common, and a positive charge possible, and being least common and rare. In a coordination compound or chelate, the central metal ion (or atom) has a positive, zero, or negative, valued oxidation state, with a positive valued oxidation state being most common, a zero valued oxidation state less common, and a negative valued oxidation state possible, and being least common and rare.

One or more multi-dentate or multifunctional ligands, or chelators, where each chelator contains at least two coordinating groups, may combine (complex) with a single metal ion, for forming a coordination compound or chelate. A coordination compound or chelate, such as that formed between one or more chelators and a metal ion, has a combined or total zero (neutral), positive, or negative, net charge. Alternatively, a single multi-dentate or multifunctional ligand, or chelator, where the chelator contains at least two coordinating groups, may combine (complex) with more than one metal ion, also for forming a coordination compound or chelate. A coordination compound or chelate, such as that formed between a single chelator and one or more metal ions, has a combined or total zero (neutral), positive, or negative, net charge. Ordinarily, one or more chelators combine (complex) with a single metal ion, rather than a single chelator combining (complexing) with more than one metal ion, for forming a coordination compound or chelate.

Each coordinating metal ion (or atom) has a definite number of coordinating groups of the one or more chelators that it can accommodate within its coordination sphere in the eventually formed coordination compound or chelate. Accordingly, the ‘coordination number’ of the metal ion or atom is the number of (coordinate covalent) bonds formed by the metal ion or atom with the electron donor or electron acceptor coordinating groups of the one or more chelators. With respect to coordination between a metal ion and one or more chelators, involving formation of a plurality of coordinate covalent bonds with the coordinating groups of the one or more chelators in the resulting coordination compound or chelate, the coordination number of the metal ion or atom is typically six or four. Lower and higher coordination numbers, for example, three and eight, respectively, of the metal ion or atom are possible. For example, as an analogy, with respect to coordination between a metal ion or atom and a plurality of individual (non-chelator type of) unidentate (one-toothed) ligands (complexing agents), involving formation of a plurality of coordinate covalent bonds with the individual ligands in the resulting coordination compound or chelate, coordination number of the metal ion or atom in the range of between two and twelve is known, with a coordination number of six, four, and eight, in this order, being the most common.

In a given coordination compound or chelate formed between at least one chelator and a metal ion, in addition to the at least two coordinate covalent bonds formed between each chelated (complexed) chelator molecule and the metal ion, a given chelator molecule may also have the capacity, with respect to electronic configuration and affinity, for bonding to, or at least interacting in a bonding-like (affinity) manner with, another chemical entity specie (uncharged or charged atom or molecule). The bonding, or the at least bonding-like (affinity) interaction, between the metal chelated (complexed) chelator molecule and the chemical entity specie is of any type and number. The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof.

An example of the preceding chelate type of chemical configuration is described in U.S. Pat. No. 4,569,794, to Smith et al., which discloses a process for separating a biologically active polypeptide or protein in the form of its precursor, from a mixture containing the precursor and impurities. The process involves contacting the precursor with a resin containing immobilized metal ions, where the precursor is a ‘hybrid protein’ composed of the biologically active polypeptide or protein covalently linked directly or indirectly to an immobilized metal ion chelating peptide bound (chelated, complexed) to the metal ions in the resin, and selectively eluting the (hybrid protein) precursor from the resin, in particular, via chemical or enzymatic treatment of the (hybrid protein) precursor. Exemplary precursor compounds are also described.

As disclosed in U.S. Pat. No. 4,569,794, the polypeptides and proteins can be naturally occurring or synthetic, and, if synthetic, they can be produced by classical solution phase, by solid phase, or by recombinant DNA methodology. The polypeptides and proteins are preferably those produced via recombinant DNA methodology. The (hybrid protein) precursor compounds are composed of two components, a biologically active polypeptide or protein, and an immobilized metal ion chelating peptide directly or indirectly joined to the metal ion by (coordinate) covalent bonding. The ‘immobilized metal ion chelating peptide’ is defined therein as an amino acid sequence that chelates immobilized divalent metal ions of metals, for example, nickel, copper, and cobalt.

Further as disclosed therein, the essential characteristics of the metal ion chelating peptide which is an element in the precursor compounds are: (1) that it chelates an immobilized metal ion, and (2) that its chelating ability is maintained when attached to a biologically active polypeptide or protein. Many peptides will chelate metal ions under conditions in which both the ion and the peptide are free from external constraints. However, when the metal ion has been immobilized, its availability for chelation is much restricted and, moreover, when the peptide which exhibits chelating activity is also joined to another entity, i.e., an active biological moiety, such as a polypeptide or protein, the potential for chelation may be reduced. Thus, the chelating peptides that participate in the precursor compositions require both of the aforementioned properties.

Suitable preferred immobilized metal ion chelating peptides are those having at least one amino acid, for example, histidine, and cysteine. The optimal length of the immobilized metal ion chelating peptide in large part will be dependent upon the number of unoccupied coordination sites on the immobilized metal ion. Iminodiacetic acid, for example, may be tridentate. Thus, depending upon the particular metal, as many as three vacant coordination sites are available for chelating (complexing) to the metal ion. Selected dipeptides thus can serve as highly efficient tridentate ligands by providing at least three potential donor atoms for chelating (complexing) with a metal ion. Normally, chelating peptides contain at least two and up to about five amino acids. Examples of specific histidine-containing immobilized metal ion chelating peptides are those of the formula His-X, in which X is selected from the group consisting of -Gly, -His, -Tyr, -Gly, -Trp, -Val, -Leu, -Ser, -Lys, -Phe, -Met, -Ala, -Glu, -Ile, -Thr, -Asp, -Asn, -Gin, -Arg, -Cys, and -Pro.

Which immobilized metal ion chelating peptide is employed in any particular situation is, of course, dependent upon a number of factors, one of which is the identity of the metal ion. For example, histidine-containing immobilized metal ion chelating peptides which chelate with Ni(II) metal ions are typically different than those which chelate with Cu(II) metal ions.

The properly designed (hybrid protein) precursor produced, for example, by recombinant DNA methodology, contains a cleavage site at the junction of the endogenous protein portion and the desired product. The cleavage site permits generation of mature product by chemical or enzymatic treatment of the hybrid protein product. Highly useful selective cleavage sites comprise a DNA sequence which codes for an amino acid or a sequence of amino acids which can be cleaved chemically or enzymatically at its C-terminal.

Examples of chemical agents useful for cleaving proteins are cyanogen bromide, 2-(2-nitrophenylsulfenyl)-3-bromo-3′-methylindolinium (BNPS-skatole), hydroxylamine, and the like. Cyanogen bromide cleaves proteins at the C-terminal of a methionine residue. Therefore, the selective cleavage site is a methionine residue itself. Hydroxylamine cleaves at the C-terminal of the moiety -Asn-Z-, in which Z is Gly, Leu, or Ala. BNPS-skatole cleaves at the C-terminal of a tryptophan residue. Examples of enzymatic agents useful for cleaving proteins are trypsin, papain, pepsin, plasmin, thrombin, enterokinase, and the like. Each effects cleavage at a particular amino acid sequence which it recognizes. Enterokinase, for example, recognizes the amino acid sequence -(Asp)ₙ-Lys-, in which n is an integer from 2 to 4.

Chelators, Coordination Compounds, Complexes, and Coordination Chemistry, in Stent Technology

There are various prior art teachings about applying chelators, coordination compounds, complexes, and coordination chemistry, to stent technology.

Hereinbelow, it is briefly, but clearly, discussed and shown that in each prior art teaching of a chelator—medical device type configuration, the chelator or chelator containing chemical (complexing agent, coupling agent, binding agent) may be directly adsorbed, adhered, coupled, bound, or bonded, to the metal surface of the medical device, ‘however’, the adsorption, adhesion, coupling, binding, or bonding, is not via coordinate covalent bonds, and therefore, is not via chelation, between coordinating groups of the chelator (complexing agent) and metal ions of the metal surface. The only possible occurrence of multiple coordinate covalent bond formation, and therefore, of chelating, is between the coordinating groups of the chelator (complexing agent) and metal ions of a chemical entity other than, and separate from, the metal surface of the medical device. In none of the various prior art teachings is there explicit or implicit description or suggestion of the coordinating groups of the chelator (complexing agent) bonding via coordinate covalent bonds, and therefore, via chelation, with metal ions of the metal surface of a medical device.

Additionally, in none of the various prior art teachings is there explicit or implicit description or suggestion of preparing or activating (for example, by oxidizing or reducing) the metal surface of a medical device (a necessary procedure for producing metal ions (typically, cations, but possibly anions) needed for forming coordinate covalent bonds, and therefore, for forming a coordinate compound via chelation) in order to even possibly enable coordination (complexation) or chelating between metal atoms of the metal surface of the medical device and the coordinating groups of the chelator (complexing agent).

Prior art teachings exist, for example, in the field of intracoronary radiation therapy (brachytherapy) with regard to the design, preparation, and implantation, of radioactive coated stents, as a well known and used therapeutic technique for attempting to prevent or/and treat restenosis, in general, and in-stent restenosis, in particular, or/and for attempting to prevent or inhibit thrombosis.

As a first example of such prior art teachings, in U.S. Pat. No. 5,871,436, to Eury, there is disclosed an implantable medical device, for example, an expandable stent, and a method of manufacturing thereof, used for delivering a dosage of radiation to a localized site, for example, a blood vessel, within a patient. The implantable medical device features a bare medical component upon which is attached a base material layer or coating, upon which is optionally bonded a spacer layer or coating, upon which is a coating of a chelator being selected for its bonding affinity (via chelation) for a specific (metallic) radioisotope.

The base material layer, and optional spacer layer, are first applied, in the form of coatings, onto the bare medical component to provide a proper foundation for the chelator, which is applied thereafter. The chelator is selected for its bonding affinity, and subsequent coordinate covalent bonding and complexing, with a specific radioisotope, for example, Ir¹⁹². It is noted that in such a configuration, the chelator is not at all complexed or chelated to the bare medical component (i.e., of the stent).

Just prior to implantation, the chelator coated medical component is immersed in a solution of the radioisotope which enables a pre-selected amount of such radioisotope to be adsorbed and (coordinately) covalently bonded (complexed, chelated) by the chelator, whereupon a coordination compound or complex is formed between the chelator and the radioisotope. The chelator-isotope combination can be chosen such that the loading is quantitative with virtually no subsequent release of the radioactive material from the implanted stent. This approach obviates any shelf life concerns related to the chelator coated stent itself and obviates the need for special handling of the chelator coated stent prior to loading. The implantable chelator coated medical component is prepared so as to readily adsorb a pre-selected amount of radioactive material and to form a sufficiently strong (coordinate covalent) bond (complex, chelate) therewith so as to substantially minimize any subsequent loss thereof upon contact with bodily fluids.

The base material is selected to both form a strong bond with the surface of the bare stent as well as with the spacer or chelator applied thereover. The base layer may comprise gold or any organic coating that contains a nucleophile, or potential nucleophile. These sites could potentially be aliphatic, or benzylic carbons a to an ester, ketone or nitrile. Alternatively, they could be alcohols, amines, ureas or thiols. Possible base layers include polyurethane, poly (ethylene-vinyl alcohols), poly (vinyl alcohols), most hydrogels and polyarcylates.

The spacer is selected to form a strong bond with the underlying base layer as well as with the chelator and serves to impart a degree of mobility to the chelator or/and to increase the number of active sites. The spacer layer is preferably attached to the base layer by nucleophilic substitution due to the degree of control afforded by such reaction. Alternatively, radical grafting processes may be employed. Possible spacer materials include α,ω-mercaptoalkylamines, diisocyanates, diacid chlorides, dialkylamines, α,ω-hydroxyalkylamines, dihydroxyalkanes (PEO), and dimercaptoalkanes.

The chelator is selected to form a (non-coordinate) covalent bond with the underlying layer, i.e., either the spacer or the base, and for having a very high binding affinity for, via coordination covalent bonding (complexing, chelating) with, the radioisotope (and not with the medical component). Such combinations of coatings are fairly tenacious, are substantially unaffected by the disinfection processes the stent is normally subjected to and have no effect on the shelf life of the stent. Possible chelator functionalities include acetates (monocarboxylic acids), acetylacetone, benzoylacetone, citric acid, 1,2-diaminocyclohexane-N,N,N′,N′-tetraacetic acid, ethlyenediamine-N,N,N′,N′-tetraacetic acid (edetic acid, EDTA), and pyridine-2,6-dicarboxylic acid.

As a second example of such prior art teachings, in U.S. Pat. No. 6,709,693, to Dinkelborg et al., there is disclosed a radioactive stent, and a method of manufacturing thereof, used for delivering a dosage of radiation to a blood vessel within a patient. The radioactive stent features a bare stent upon which is a coating of an adhesive which is in the form of either a complexing agent (chelator) by itself, or includes a complexing agent (chelator) in its structure, that is complexed or chelated (exclusively) to a radioactive isotope of a metal, wherein the complexing agent (chelator) is not at all complexed or chelated to the bare stent. The adhesive is a complexing peptide or a peptide capable of being activated for complexing with a radioactive metal, or is a complexing fat or a fat capable of being activated for complexing with a radioactive metal, or is gold coated with a thiol-group-containing complexing agent (chelator) which is capable of complexing with a radioactive metal. In each embodiment, the adhesive or complexing agent (chelator) is in no way or manner complexed or chelated to the bare stent.

As disclosed in U.S. Pat. No. 6,709,693, a first embodiment of the process for preparing the radioactive stent features reacting a radioactive isotope with an adhesive that is a peptide, a fat, or gold used in combination with a thiol-group-containing complexing agent, followed by coating the bare stent with the radiolabeled adhesive. A second embodiment of the process for preparing the radioactive stent features coating a non-radioactive bare stent with an adhesive that is a peptide, a fat or gold used in combination with a thiol-group-containing complexing agent, followed by coating the adhesive coated stent with a radioactive isotope by placing the adhesive coated stent into a solution of the radioactive isotope. The overall process for preparing the radioactive stent is based on using techniques of chemical reduction, chemical precipitation, or, electrochemical deposition via electroplating (external electrolysis) or cementation (internal electrolysis).

It is highly notable and important to clearly point out, for the purpose of clearly distinguishing prior art teachings from teachings of the present invention, as illustratively described and claimed hereinbelow, that in each of the disclosures of U.S. Pat. No. 5,871,436 and U.S. Pat. No. 6,709,693, the chelator (complexing agent) has two specific and exclusive functions: (1) for being adsorbed, adhered, coupled, bound, or bonded (via non-coordinate covalent bonds), onto the bare (metal) surface, either by itself (U.S. Pat. No. 5,871,436; U.S. Pat. No. 6,709,693) or via a spacer compound (U.S. Pat. No. 5,871,436), for forming a non-chelate type of coating on the bare metal surface, and (2) for adsorbing an amount of a radioisotope, for forming one or more sufficiently strong (coordinate covalent) bonds therewith, for producing a coordination compound (coordination complex) between the chelator (complexing agent) and the radioisotope, thus enabling production of a radioactive implantable medical device usable for delivering a dosage of radiation to a blood vessel within a patient.

Although in each radioactive implantable medical device the chelator or chelator containing chemical (complexing agent) may be directly adsorbed, adhered, coupled, bound, or bonded, to the bare metal surface of the device, the adsorption, adhesion, coupling, binding, or bonding, is not via coordinate covalent bonds, and therefore, is not via chelation, between coordinating groups of the chelator (complexing agent) and metal ions of the bare metal surface. The only possible occurrence of multiple coordinate covalent bond formation, and therefore, of chelating, is between the coordinating groups of the chelator (complexing agent) and the metal ions of the radioisotope. Nowhere in either disclosure is there explicit or implicit description or suggestion of the coordinating groups of the chelator (complexing agent) bonding via coordinate covalent bonds, and therefore, via chelation, with metal ions of the bare metal surface of the implantable medical device.

Additionally, nowhere in either disclosure is there explicit or implicit description or suggestion of preparing or activating (for example, by oxidizing or reducing) the metal surface of the medical device (a necessary procedure for producing metal ions (typically, cations, but possibly anions) needed for forming coordinate covalent bonds, and therefore, for forming a coordinate compound via chelation) in order to even possibly enable coordination (complexation) or chelating between metal atoms of the bare metal surface of the medical device and the coordinating groups of the chelator (complexing agent).

In PCT Int'l. Pat. Appl. Pub. No. WO 2004/037120, published May 6, 2004, entitled “Implantable Medical Devices Using Zinc”, there are disclosed teachings of an implantable medical device, for example, a stent, a graft, or a stent-graft, which is coupled (bound) with at least one zinc-containing component (e.g., elemental zinc, ionic zinc, zinc compound, zinc complex, zinc chelate, zinc-containing matrix or gel, combinations thereof, or any other zinc-containing component or substance), and methods using thereof, to inhibit plaque formation, enhance elastin production, or the like. Disclosed therein are numerous different chemical or/and physical techniques, means, and embodiments, for ‘coupling’ or ‘binding’ the zinc-containing component to a surface of an implantable medical device made of metal, non-metal (e.g., plastic, ceramic), or a combination thereof (e.g., a composite material).

Among those, in one embodiment (as illustratively described therein, with particular reference to FIGS. 7 and 8, and Example 1, therein), coupling (binding) the zinc-containing component to the implantable medical device includes coupling (binding) a zinc chelator (coupling or binding agent) to the surface of the medical device and releasably coupling (binding, via ‘chelating’) the zinc-containing component (e.g., via zinc cations) to the chelator. Optionally, the method may further include polymerizing the chelator, for the purpose of increasing absolute or relative amount of the zinc-containing component releasably coupled or bound to the medical device.

For preparing the preceding embodiment, allylamine is first bound to the surface of the medical device, in order to generate reactive amines (upon the surface), which are then coupled to aspartate via an amide linkage. Each aspartate then serves as the coupling (binding, complexing, chelating) agent to couple (bind, complex, chelate) the zinc-containing component (e.g., zinc cations).

In WO 2004/037120, fundamentally, the same as in the prior art teachings of the disclosures of U.S. Pat. No. 5,871,436 and U.S. Pat. No. 6,709,693, in the zinc chelator—medical device type configuration, the chelator or chelator containing chemical (complexing agent, coupling agent, binding agent), e.g., aspartate, may be directly adsorbed, adhered, coupled, bound, or bonded, to the metal surface of the medical device, ‘however’, the adsorption, adhesion, coupling, binding, or bonding, is not via coordinate covalent bonds, and therefore, is not via chelation, between coordinating groups of the chelator (complexing agent), e.g., aspartate, and metal ions of the metal surface. The only possible occurrence of multiple coordinate covalent bond formation, and therefore, of chelating, is between the coordinating groups of the chelator (complexing agent), e.g., aspartate, and metal ions of a chemical entity, in particular, zinc ions (cations), other than, and separate from, the metal surface of the medical device.

Moreover, nowhere in WO 2004/037120 is there explicit or implicit description or suggestion of the coordinating groups of the chelator (complexing agent), e.g., aspartate, bonding via coordinate covalent bonds, and therefore, via chelation, with metal ions of the metal surface of the medical device. Additionally, nowhere in WO 2004/037120 is there explicit or implicit description or suggestion of preparing or activating (for example, by oxidizing or reducing) the metal surface of the medical device (a necessary procedure for producing metal ions (typically, cations, but possibly anions) needed for forming coordinate covalent bonds, and therefore, for forming a coordinate compound via chelation) in order to even possibly enable coordination (complexation) or chelating between metal atoms of the metal surface of the medical device and the coordinating groups of the chelator (complexing agent).

In U.S. Pat. No. 6,264,596, to Weadock, there are disclosed devices and methods for rendering an intravascular stent radioactive in-situ, after stent placement, in particular, for inhibiting restenosis of blood vessels. A stent is provided having a tubular body and a first substance immobilized on the body. The first substance preferably has a high and selective affinity for a second substance which can be radioactive, cytotoxic, or thrombolytic. Therein, it is stated that “one complementary binding pair of substances suitable for use with the present invention is the avidin/biotin pair”, and that the “biotin can be immobilized on a metallic stent by chelating agents which have affinity for metals, silanes, or other forms of molecular grafting known by those skilled in the art”. It is also stated that “another complementary pair of substances suitable for practicing the present invention is the protamine/heparin pair”, and that “protamine can be immobilized on a metallic stent through use of chelating agents having an affinity for the metal and protamine or through plasma deposition”.

Again, however, fundamentally, the same as in the prior art teachings in the disclosures of U.S. Pat. No. 5,871,436, U.S. Pat. No. 6,709,693, and WO 2004/037120, nowhere in U.S. Pat. No. 6,264,596 is there description or suggestion regarding coordinating groups of the chelator (complexing agent), e.g., biotin or protamine, bonding via coordinate covalent bonds, and therefore, via chelation, with metal ions of the metal surface of the stent. Additionally, nowhere in U.S. Pat. No. 6,264,596 is there description or suggestion of preparing or activating the metal surface of the stent in order to even possibly enable coordination (complexation) or chelating between metal atoms of the metal surface of the stent and the coordinating groups of the chelator (complexing agent), i.e., biotin or protamine.

Based upon the above described limitations and shortcomings of prior art teachings of systemic pharmacological techniques, brachytherapy techniques, and various other, techniques for customizing or/and optimizing physical parameters of bare stent design and construction, as well as drug coated stent and drug eluting stent technologies, failing to provide a sufficiently effective, consistent, robust, and safe, solution to restenosis, in general, and in-stent restenosis, in particular, there is a strong need for continuing research, development, testing, and use, of new techniques for preventing or/and treating restenosis, as well as for preventing or/and treating thrombosis associated with restenosis.

Currently, there is clearly an international widespread consensus of the need for further research, development, clinical testing and long term follow-up investigational studies thereof, of all the relevant aspects and parameters relating to drug coated and drug eluting stents, in particular, and relating to drug coated and drug eluting medical implants or medical implant components, in general. Particular aspects which need to be focused on have to do with the types and physicochemical properties, characteristics, and behaviors, of coatings coated onto medical implants or medical implant components, such as stents, as an important part of producing drug coated or drug eluting medical implant components, devices, and systems. Especially, regarding possible alternatives or substitutions, such as ‘polymer-free’ based types of coatings, to currently known and applied ‘polymer’ based types of coatings.

There is thus a need for, and it would be highly advantageous to have a medical device featuring a medical implant or medical implant component having a metal surface to which is bound a chemical entity via a chelator chelated to the metal surface. Moreover, there is further need for having a method of manufacturing the medical implant device thereof, a medical implant system including the medical implant device thereof, a method of implanting the medical implant device thereof, a method of preventing or/and treating a medical condition of a subject using the medical implant device thereof, a chelate type of coordination compound including a drug or a biological moiety, and a medical device featuring a medical implant having a metal surface to which is chelated a chelator in a chelate configuration.

### SUMMARY OF THE INVENTION

The present invention relates to a medical device featuring a medical implant or medical implant component having a metal surface to which is bound a chemical entity via a chelator chelated to the metal surface. The present invention further particularly relates to a method of manufacturing the medical implant device thereof, a medical implant system including the medical implant device thereof, a method of implanting the medical implant device thereof, a method of preventing or/and treating a medical condition of a subject using the medical implant device thereof, a chelate type of coordination compound including a drug, and a medical device featuring a medical implant or medical implant component having a metal surface to which is chelated a chelator in a chelate configuration.

An exemplary medical implant or medical implant component having a metal surface which is particularly suitable for applying the present invention is a stent. Chemical entities which are suitable for applying the present invention are essentially any of a wide variety of different categories and types of chemical compounds, for example, a drug, a biological moiety, a linker or spacer capable of binding a drug or a biological moiety, and a linker or spacer to which a drug or a biological moiety is bound. Exemplary biological moiety type chemical entity species of the chemical entity which are suitable for implementing the present invention are proteins, lipids (fats), sugars, nucleic acids, antibodies, cells, cellular structures, cellular components, and combinations thereof.

In an exemplary preferred embodiment, the chelator is chelated to the metal surface of the medical implant or medical implant component, for example, a stent, in a form of a coating, whereupon the chemical entity (linker-drug or linker-biological moiety) bound to the metal surface via the chelator coating results in the formation of a drug (or a biological moiety) coated or drug (or a biological moiety) eluting medical implant device, for example, a drug (or a biological moiety) coated or drug (or a biological moiety) eluting stent, wherein activity of the bound chemical entity exhibits efficacy for preventing or/and treating a medical condition, disease, or ailment, such as restenosis, in general, in-stent restenosis, in particular, or/and thrombosis, in a human or animal subject.

Thus, according to the present invention, there is provided a medical device comprising a medical implant component having a metal surface (M) to which is bound a chemical entity (X) via a chelator (C) chelated to the metal surface in an (M)-(C)-(X) configuration.

According to another aspect of the present invention, there is provided a medical device comprising a medical implant component having a surface to which is bound a chemical at a surface concentration of greater than 100 picograms (pg) per cm².

According to another aspect of the present invention, there is provided a method of manufacturing a medical device comprising binding to a metal surface (M) of a medical implant component a chemical entity (X) via a chelator (C) in an (M)-(C)-(X) configuration.

According to another aspect of the present invention, there is provided a medical implant system comprising: (a) a medical implant component having a metal surface (M) to which is bound a chemical entity (X) via a chelator (C) chelated to the metal surface in an (M)-(C)-(X) configuration; and (b) a delivery device for delivering the medical implant component to a pre-determined position in a subject.

According to another aspect of the present invention, there is provided a method of implanting a medical device comprising, implanting in a subject in need thereof a medical device which comprises a medical implant component having a metal surface (M) to which is bound a chemical entity (X) via a chelator (C) chelated to the metal surface in an (M)-(C)-(X) configuration.

According to another aspect of the present invention, there is provided a method of implanting a medical device comprising, implanting in a subject in need thereof a medical device which comprises a medical implant component having a surface to which is bound a chemical at a surface concentration of greater than 100 picograms (pg) per cm².

According to another aspect of the present invention, there is provided a method of preventing or/and treating a medical condition of a subject, comprising implanting in the subject a medical device which comprises a medical implant component having a metal surface (M) to which is bound a chemical entity (X) via a chelator (C) chelated to the metal surface in an (M)-(C)-(X) configuration, such that activity of the bound chemical entity exhibits an efficacy for preventing or/and treating the medical condition. According to another aspect of the present invention, there is provided a chelate type of coordination compound comprising a structure of general formula: (C)-(Y), wherein (C) is a chelator and (Y) is selected from the group consisting of (i) a drug chelated to the chelator or a biological moiety chelated to the chelator, and, (ii) a linker having a first part chelated to the chelator and having a second part bonded to a drug or a biological moiety.

According to another aspect of the present invention, there is provided a medical device comprising a medical implant component having a metal surface (M) to which is chelated a chelator (C).

### BRIEF DESCRIPTION OF THE DRAWINGS

The present invention is herein described, by way of example only, with reference to the accompanying drawings. With specific reference now to the drawings in detail, it is stressed that the particulars shown are by way of example and for purposes of illustrative description of the preferred embodiments of the present invention only, and are presented in the cause of providing what is believed to be the most useful and readily understood description of the principles and conceptual aspects of the present invention. In this regard, no attempt is made to show structural details of the present invention in more detail than is necessary for a fundamental understanding of the invention, the description taken with the drawings making apparent to those skilled in the art how the several forms of the invention may be embodied in practice. In the drawings:

FIG. 1 is a conceptual ‘micro (atomic, molecular, compound)/macro (coating) level’ schematic diagram illustrating a cut-away side view of characteristic features of exemplary preferred embodiments of the ‘metal chelated surface’ medical implant device, featuring a metal surface (M) of a medical implant or medical implant component to which is bound a chemical entity (X) via a chelator (C) chelated to the metal surface in an (M)-(C)-(X) configuration, and of an exemplary application thereof for selectively cleaving or breaking the various different types of bonding or bonding-like (affinity) interaction, leading to release (elution) and migration of one or more bound chemical species (d1, d2, d3, d4), such as anti-restenosis or/and anti-thrombosis drugs, in a stented blood vessel, in accordance with the present invention;

FIG. 2 is essentially the same as FIG. 1, but illustrating different alternative types of configurations of coordinate covalent bonding, between chelator molecules of the chelator (C) and metal ions or atoms of the metal surface (M), in the chelate type of coordination compound configurations, in accordance with the present invention;

FIG. 3 is the same as FIG. 1, additionally illustrating the selective release (elution) and migration of the one or more bound chemical species (d1, d2, d3, d4), such as anti-restenosis or/and anti-thrombosis drugs, in the stented blood vessel, in accordance with the present invention; and

FIG. 4 is a conceptual ‘micro (atomic, molecular, compound)/macro (coating) level’ schematic diagram illustrating a cut-away side view of characteristic features of an exemplary preferred embodiment of the ‘metal chelated surface’ medical implant device, featuring a metal surface (M) of a medical implant or medical implant component to which is chelated a chelator (C) in an (M)-(C) configuration, and of an exemplary application thereof for selectively binding, via chelation (complexation), of ‘free’ metal ions (w1 and w2) involved in the onset or/and progress of restenosis or/and thrombosis processes, which are flowing through a stented blood vessel, in accordance with the present invention.

### DESCRIPTION OF THE PREFERRED EMBODIMENTS

The present invention relates to a medical device featuring a medical implant or medical implant component having a metal surface to which is bound a chemical entity via a chelator chelated to the metal surface. The present invention further particularly relates to a method of manufacturing the medical implant device thereof, a medical implant system including the medical implant device thereof, a method of implanting the medical implant device thereof, a method of preventing or/and treating a medical condition of a subject using the medical implant device thereof, a chelate type of coordination compound including a drug, and a medical device featuring a medical implant or medical implant component having a metal surface to which is chelated a chelator in a chelate configuration.

An exemplary medical implant or medical implant component having a metal surface which is particularly suitable for applying the present invention is a stent. Chemical entities which are suitable for applying the present invention are essentially any of a wide variety of different categories and types of chemical compounds, for example, a drug, a biological moiety, a linker or spacer capable of binding a drug or a biological moiety, and a linker or spacer to which a drug or a biological moiety is bound. Exemplary biological moiety type chemical entity species of the chemical entity which are suitable for implementing the present invention are proteins, lipids (fats), sugars, nucleic acids, antibodies, cells, cellular structures, cellular components, and combinations thereof.

In an exemplary preferred embodiment, the chelator is chelated to the metal surface of the medical implant or medical implant component, for example, a stent, in a form of a coating, whereupon the chemical entity (linker-drug or linker-biological moiety) bound to the metal surface via the chelator coating results in the formation of a drug (or a biological moiety) coated or drug (or a biological moiety) eluting medical implant device, for example, a drug (or a biological moiety) coated or drug (or a biological moiety) eluting stent, wherein activity of the bound chemical entity exhibits efficacy for preventing or/and treating a medical condition, disease, or ailment, such as restenosis, in general, in-stent restenosis, in particular, or/and thrombosis, in a human or animal subject.

A first main aspect of novelty and inventiveness of the present invention is provision of an implantable medical device characterized by including a medical implant component having a metal surface (M) to which is bound a chemical entity (X) via a chelator (C) chelated to the metal surface in an (M)-(C)-(X) configuration.

A second main aspect of the present invention is provision of an implantable medical device characterized by including a medical implant component having a surface to which is bound a chemical at a surface concentration of greater than 100 picograms (pg) per cm².

A third main aspect of the present invention is provision of a method of manufacturing an implantable medical device characterized by including the step of binding to a metal surface (M) of a medical implant component a chemical entity (X) via a chelator (C) in an (M)-(C)-(X) configuration.

A fourth main aspect of the present invention is provision of a medical implant system characterized by including: (a) a medical implant component having a metal surface (M) to which is bound a chemical entity (X) via a chelator (C) chelated to the metal surface in an (M)-(C)-(X) configuration; and (b) a delivery device for delivering the medical implant component to a pre-determined position in a subject.

A fifth main aspect of the present invention is provision of a method of implanting a medical device characterized by including the step of implanting in a subject in need thereof a medical device which includes a medical implant component having a metal surface (M) to which is bound a chemical entity (X) via a chelator (C) chelated to the metal surface in an (M)-(C)-(X) configuration.

A sixth main aspect of the present invention is provision of a method of implanting a medical device characterized by including the step of implanting in a subject in need thereof a medical device which includes a medical implant component having a surface to which is bound a chemical at a surface concentration of greater than 100 picograms (pg) per cm².

A seventh main aspect of the present invention is provision of a method of preventing or/and treating a medical condition of a subject characterized by including the step of implanting in the subject a medical device which includes a medical implant component having a metal surface (M) to which is bound a chemical entity (X) via a chelator (C) chelated to the metal surface in an (M)-(C)-(X) configuration, such that activity of the bound chemical entity exhibits an efficacy for preventing or/and treating the medical condition.

An eighth main aspect of the present invention is provision of a chelate type of coordination compound characterized by having a configuration of general formula: (C)-(Y), wherein (C) is a chelator and (Y) is selected from the group consisting of (i) a drug chelated to the chelator or a biological moiety chelated to the chelator, and, (ii) a linker having a first part chelated to the chelator and having a second part bonded to a drug or a biological moiety.

A ninth main aspect of the present invention is provision of a medical device characterized by including a medical implant component having a metal surface (M) to which is chelated a chelator (C) in a chelate configuration.

Accordingly, the present invention includes several aspects of novelty and inventiveness over the relevant prior art in the field of medical implant technology, in general, and especially in the sub-fields of drug coated stent and drug eluting stent technologies, relating to the need for finding and providing a sufficiently effective, consistent, robust, and safe, solution to restenosis, in general, and in-stent restenosis, in particular. More particularly, with respect to aspects focusing on the types and physicochemical properties, characteristics, and behaviors, of coatings coated onto medical implants or medical implant components, such as stents, as an important part of producing drug coated or drug eluting medical implant components, devices, and systems. Especially, regarding possible alternatives or substitutions, such as ‘polymer-free’ based types of coatings, to currently known and applied ‘polymer’ based types of coatings.

It is to be understood that the present invention is not limited in its application to the details of type, composition, construction, arrangement, order, and number, of structures, components, elements, and configurations, and, peripheral equipment, utilities, accessories, chemical reagents, and materials, or to the details of the order or sequence, number, of procedures, steps, and sub-steps, of operation, of the various preferred embodiments set forth in the following illustrative description, accompanying drawings, and examples, unless otherwise specifically stated herein.

For example, in the following description, implementation of the present invention is exemplified by way of application to a medical device in the form of a medical implant or medical implant component, for example, a stent, having a metal surface to which is bound a chemical entity, for example, a drug, or a biological moiety, a linker or spacer capable of binding a drug or a biological moiety, or/and, a linker or spacer to which a drug or a biological moiety is bound, via a chelator chelated to the metal surface, wherein activity of the bound chemical entity exhibits efficacy for preventing or/and treating a medical condition, disease, or ailment, such as restenosis, in general, in-stent restenosis, in particular, or/and thrombosis, in a human or animal subject. In a non-limiting manner, the scope of implementation of the present invention clearly includes applications to various other medical devices in the form of a medical implant or medical implant component, which can have a metal surface, for example, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a prosthesis, a plate, a joint, a fin, a screw, a spike, a wire, a filament, a thread, an anchor, or a bone fixation element, among other exemplary medical devices.

Additionally, in the following description, implementation of the present invention is exemplified by way of application of the medical device for preventing or/and treating a medical condition, disease, or ailment, such as restenosis, in general, in-stent restenosis, in particular, or/and thrombosis, in a human or animal subject. In a non-limiting manner, the scope of implementation of the present invention clearly includes applications of the medical device for preventing or/and treating various other medical conditions, diseases, or ailments.

Accordingly, the present invention is capable of other embodiments and of being practiced or carried out in various ways. Although structures, components, and elements, and, peripheral equipment, utilities, accessories, chemical reagents, and materials, and, procedures, steps, sub-steps, similar or equivalent to those illustratively described herein can be used for practicing or testing the present invention, suitable structures, components, and elements, and, peripheral equipment, utilities, accessories, chemical reagents, and materials, and procedures, steps, sub-steps, are illustratively described herein.

It is also to be understood that all technical and scientific words, terms, or/and phrases, used herein throughout the present disclosure have either the identical or similar meaning as commonly understood by one of ordinary skill in the art to which this invention belongs, unless otherwise specifically defined or stated herein. Phraseology, terminology, and, notation, employed herein throughout the present disclosure are for the purpose of description and should not be regarded as limiting. For example, throughout the disclosure of the present invention, reference is made to a medical implant or medical implant component having a metal surface (M) to which is bound a chemical entity (X) via a chelator (C) chelated to the metal surface in an (M)-(C)-(X) configuration, in order to illustrate implementation of the present invention.

It is to be fully understood that throughout the disclosure of the present invention, the term ‘medical implant’ generally corresponds to an entire or whole medical implant, for example, an entire or whole stent, or, an entire or whole prosthesis, and the term ‘medical implant component’ generally corresponds to ‘at least’ a section of at least a part or component of an entire or whole medical implant. Accordingly, in a non-limiting manner, a medical implant component may also correspond to an entire or whole medical implant. Thus it is to be fully understood that the present invention is clearly applicable to at least a section of at least a part or component of an entire or whole medical implant, as well as to an entire or whole medical implant.

Moreover, all technical and scientific words, terms, or/and phrases, introduced, defined, described, or/and exemplified, in the above Background section, are equally or similarly applicable in the illustrative description of the preferred embodiments, examples, and appended claims, of the present invention. Additionally, as used herein, the term ‘about’ refers to ±10% of the associated value. Additionally, as used herein, the phrase ‘room temperature’ refers to a temperature in a range of between about 20° C. and about 25° C.

Components, elements, and configurations, and, peripheral equipment, utilities, accessories, chemical reagents, and materials, procedures, steps, sub-steps, as well as operation, and implementation, of exemplary preferred embodiments, alternative preferred embodiments, specific configurations, and, additional and optional aspects, characteristics, or features, thereof, according to the present invention, are better understood with reference to the following illustrative description and accompanying drawings. Throughout the following illustrative description and accompanying drawings, same reference numbers or/and letters refer to same structures, components, elements, and configurations.

In the following illustrative description of the present invention, included are main or principal structures, components, elements, and configurations, and, peripheral equipment, utilities, accessories, chemical reagents, and materials, and functions thereof, and, main or principal procedures, steps, and sub-steps, needed for sufficiently understanding proper ‘enabling’ utilization and implementation of the disclosed invention. Accordingly, description of various possible preliminary, intermediate, minor, or/and optional, structures, components, elements, and configurations, and, peripheral equipment, utilities, accessories, chemical reagents, and materials, or/and functions thereof, or/and, procedures, steps, or/and sub-steps, which are readily known by one of ordinary skill in the art, which are available in the prior art or/and technical literature relating to the present invention, are at most only briefly indicated herein.

According to a main aspect of the present invention, there is provided an implantable medical device characterized by including a medical implant or medical implant component having a metal surface (M) to which is bound a chemical entity (X) via a chelator (C) chelated to the metal surface in an (M)-(C)-(X) configuration, herein, also referred to as an (M)-(C)-(X) chelate type of coordination compound configuration.

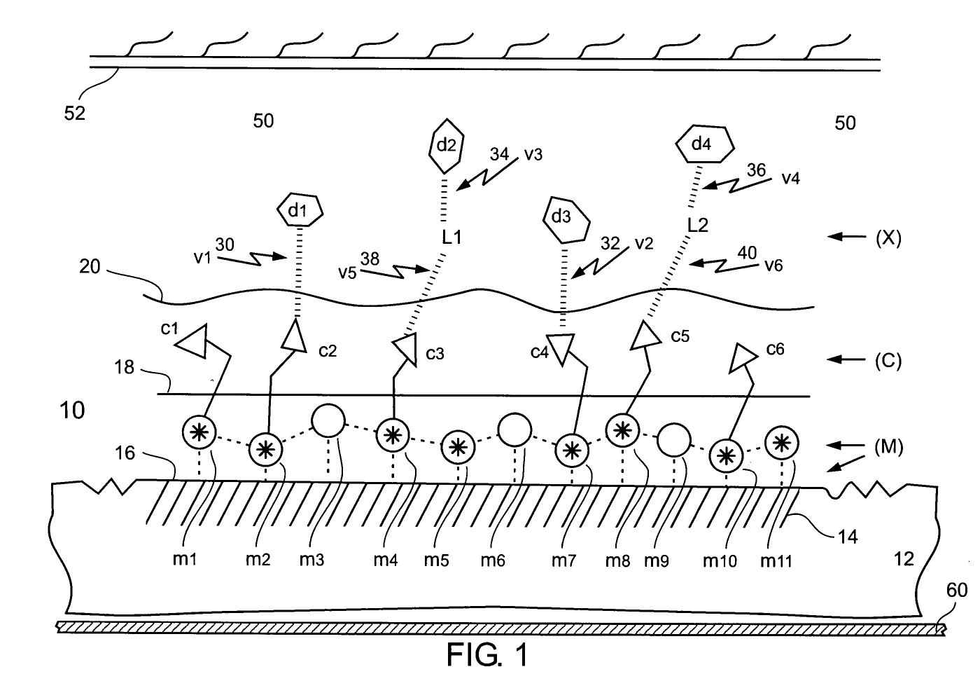

Referring now to the drawings, FIG. 1 is a conceptual ‘micro (atomic, molecular, compound)/macro (coating) level’ schematic diagram illustrating a cut-away side view of characteristic features of exemplary preferred embodiments of the ‘metal chelated surface’ medical implant device, herein, equivalently referred to as medical implant device 10, or as ‘metal chelated surface’ medical device 10, or for brevity, as medical device 10, featuring a metal surface (M) of a medical implant component 12 to which is bound a chemical entity (X) via a chelator (C) chelated to the metal surface (M) in an (M)-(C)-(X) configuration.

In ‘metal chelated surface’ medical device 10, as illustrated in FIG. 1, medical implant component 12 generally corresponds to, and is generally representative of, at least a section of at least a part or component having a metal surface (M), of an entire or whole medical implant, such as a stent or a prosthesis.

An exemplary part or component having a metal surface (M), of a stent, is a metal wire, a metal filament, or a metal thread, or, alternatively, a metal film, a metal plating, or a metal coating, deposited upon at least a section of another non-metal or metal part or component of the stent. Accordingly, as an example, medical implant component 12 can generally correspond to, and be generally representative of, at least a section of at least a metal wire, a metal filament, or a metal thread, of a stent, or, alternatively, at least a section of a metal film, a metal plating, or a metal coating, deposited upon at least a section of another non-metal or metal part or component of a stent.

An exemplary part or component having a metal surface (M), of a prosthesis, is a metal plate, a metal joint, a metal fin, a metal screw, a metal spike, a metal wire, a metal filament, a metal thread, a metal anchor, or another metallic bone fixation element, or, a metal film, a metal plating, or a metal coating, deposited upon at least a section of another non-metal or metal part or component of the prosthesis. Accordingly, as an example, medical implant component 12 can generally correspond to, and be generally representative of, at least a section of at least a metal plate, a metal joint, a metal fin, a metal screw, a metal spike, a metal wire, a metal filament, a metal thread, a metal anchor, or another metallic bone fixation element, of a prosthesis, or alternatively, at least a section of at least a metal film, a metal plating, or a metal coating, deposited upon at least a section of another non-metal or metal part or component of a prosthesis.

Within the scope of the present invention, in a non-limiting manner, it is to be fully understood that medical implant component 12 may also generally correspond to, and be representative of, an entire or whole part or component having a metal surface (M), of a medical implant, such as a stent or a prosthesis, or, alternatively, an entire or whole medical implant having a metal surface (M), such as an entire or whole stent having a metal surface (M), or an entire or whole prosthesis having a metal surface (M).

Additionally, in a non-limiting manner, it is to be fully understood that metal surface (M) represents an external (outer) side or/and an internal (inner) side of medical implant component 12. For example, in the case that medical implant component 12 represents at least a section of a metal wire, a metal filament, or a metal thread, of a stent (for example, which is deliverable to, and implantable at, a pre-determined position in a subject, for example, inside the cavity of a blood vessel, for being longitudinally extended along the side of the blood vessel wall), having an external (outer or abluminal) side (for example, facing the blood vessel wall), and an internal (inner or luminal) side (for example, facing the hollow inside or lumen of the stent), then metal surface (M) represents an external (outer or abluminal) side or/and an internal (inner or luminal) side of at least a section of the metal wire, metal filament, or metal thread, of the stent. For illustrative purposes only, in a non-limiting manner, as an example, as shown illustrated in FIG. 1, metal surface (M) of medical implant component 12 represents an external (outer or abluminal) side of at least a section of a metal wire, a metal filament, or a metal thread, of a stent (for example, which is deliverable to, and implantable at, a pre-determined position in a subject, for example, inside the cavity, for example, cavity 50, of a blood vessel, for being longitudinally extended along the side of the blood vessel wall, for example, blood vessel wall 52), having an external (outer or abluminal) side facing blood vessel wall 52.

Metal surface (M) of medical implant component 12 of ‘metal chelated surface’ medical device 10, includes uppermost or exposed surface metal ions and atoms (in FIG. 1, schematically represented by star containing circles and empty circles m1-m11), immediately beneath which is a sub-surface region 14 of metal atoms. An interface layer or continuum 16 lies between exposed surface metal ions and atoms m1-m11 and sub-surface region 14 of metal atoms. Various types of ion-ion, atom-atom, and ion-atom, inter-ionic and inter-atomic electronic interactions (in FIG. 1, generally, schematically represented by dashed lines in metal surface (M)), including, for example, metallic bonding, dipole-dipole interactions, attraction (affinity), repulsion, polarization, and combinations thereof, exist among exposed surface metal ions and atoms m1-m11 themselves, as well as among exposed surface metal ions and atoms m1-m11 and metal atoms of sub-surface region 14.

For a given set of parameters of previous and current physicochemical treatments or/and conditions, in the current or instant population of exposed surface metal ions and atoms m1-m11, of metal surface (M), there exists a sub-population of exposed surface metal ions and atoms (star containing circles m1, m2, m4, m7, m8, and m10) each of which is charged (cationic or anionic), uncharged (neutral), or polarized, and is chelated (complexed) to a chelator molecule (in FIG. 1, schematically represented by empty triangles c1, c2, c3, c4, c5, and c6, respectively) of chelator (C), in the form of a chelate type of coordination compound (metal complex, metal ion complex, coordination complex, chelate complex, chelate ring, or, chelate) configuration. Such metal surface (M)-chelator (C) chelate type of coordination compound configurations can be symbolized as m1-c1, m2-c2, m4-c3, m7-c4, m8-c5, and m10-c6, respectively.

Chelator (C) is composed of essentially any single type of molecules, or combination of two or more single types of molecules, wherein each molecule has at least two coordinating (complexing, chelating) groups in its structure and functions as a multi-dentate or multifunctional ligand for complexing or chelating to (coordinating with) at least a single metal ion (or atom) of a metal surface, such as metal surface (M) of medical implant component 12 of ‘metal chelated surface’ medical device 10, via at least two coordinate covalent bonds, for forming a chelate type of coordination compound.

Each metal surface (M)-chelator (C) chelate type of coordination compound configuration, m1-c1, m2-c2, m4-c3, m7-c4, m8-c5, and m10-c6, is characterized by having at least two coordinate covalent bonds between the corresponding chelated (complexed) exposed surface metal ion or atom m1, m2, m4, m7, m8, or m10, respectively, of metal surface (M), and at least two coordinating groups of the corresponding chelator molecule c1, c2, c3, c4, c5, or c6, respectively, of chelator (C). For each chelate type of coordination compound configuration, m1-c1, m2-c2, m4-c3, m7-c4, m8-c5, and m10-c6, the at least two coordinate covalent bonds are generally represented by a single bi-directional or angled line extending between each chelated (complexed) exposed surface metal ion or atom m1, m2, m4, m7, m8, or m10, of metal surface (M), and each corresponding chelator molecule c1, c2, c3, c4, c5, or c6, respectively, of chelator (C).

There also exists a sub-population of non-chelated (non-complexed) exposed surface metal ions (star containing circles m5 and m11) of metal surface (M), each of which is charged (cationic or anionic) and not chelated (complexed) to any chelator molecule, for example, c1-c6, of chelator (C). There also exists a sub-population of non-chelated (non-complexed) exposed surface metal atoms (empty circles m3, m6, and m9) each of which is uncharged (neutral) or at most polarized and not chelated (complexed) to any chelator molecule, for example, c1-c6, of chelator (C). Within the scope of the present invention, in a non-limiting manner, it is to be fully understood that for a different given set of parameters of previous and current physicochemical treatments or/and conditions, metal surface (M) of medical implant component 12 of ‘metal chelated surface’ medical device 10, can include any number of different types of sub-populations and configurations of chelated (complexed) and non-chelated exposed surface metal ions and atoms. An interface layer or continuum 18 lies between chelator molecules c1-c6 of chelator (C) and exposed surface metal ions and atoms m1-m11 of metal surface (M).

Accordingly, as just illustratively described with reference to FIG. 1, ‘metal chelated surface’ medical device 10 includes a medical implant component 12 having a metal surface (M) to which is chelated a chelator (C) in an (M)-(C) chelate type of coordination compound configuration. Thus, in accordance with another main aspect of the present invention, there is provision of a medical device characterized by including a medical implant component having a metal surface (M) to which is chelated a chelator (C).

In the current or instant population of metal chelated (complexed) chelator molecules c1-c6 of chelator (C), there exists a sub-population of metal chelated (complexed) chelator molecules, c2, c3, c4, and c5, each of which is bonded to, or at least interacts in a bonding-like (affinity) manner with, a chemical entity specie (uncharged or charged atom or molecule) (in FIG. 1, schematically represented by hexagonal d1, L1, hexagonal d3, or L2, respectively) of chemical entity (X). Such metal surface (M)-chelator (C)-chemical entity (X) chelate type of coordination compound configurations can be symbolized as m2-c2-X (wherein X=d1), m4-c3-X (wherein X=L1), m7-c4-X (wherein X=d3), and m8-c5-X (wherein X=L2), respectively.

For each metal surface (M)-chelator (C)-chemical entity (X) chelate type of coordination compound configuration, m2-c2-X (wherein X d1), m4-c3-X (wherein X=L1), m7-c4-X (wherein X=d3), and m8-c5-X (wherein X=L2), the bonding, or the at least bonding-like (affinity) interaction, between the corresponding metal chelated (complexed) chelator molecule c2, c3, c4, and c5, respectively, of chelator (C), and the corresponding chemical entity specie d1, L1, d3, and L2, respectively, of chemical entity (X), is of any type and number. The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof. For each metal surface (M)-chelator (C)-chemical entity (X) chelate type of coordination compound configuration, m2-c2-d1, m4-c3-L1, m7-c4-d3, and m8-c5-L2, such bonding, or at least bonding-like (affinity) interaction, is generally represented by a single (sideless) ladder of dashes extending between the corresponding metal chelated (complexed) chelator molecule c2, c3, c4, and c5, respectively, of chelator (C), and the corresponding chemical entity specie d1, L1, d3, and L2, respectively, of chemical entity (X).

There also exists a sub-population of metal chelated (complexed) chelator molecules, c1 and c6, each of which is not bonded to any chemical entity specie, for example, d1, L1, d3, or L2, of chemical entity (X). An interface layer or continuum 20 lies between chemical entity species d1, L1, d3, and L2, of chemical entity (X), and metal chelated (complexed) chelator molecules c1-c6 of chelator (C).

In the current or instant population of (chelator bonded or interacting) chemical entity species d1, L1, d3, and L2, of chemical entity (X), there exists a sub-population of (chelator bonded or interacting) chemical entity species, L1 and L2, each of which is additionally bonded to, or at least interacts in a bonding-like (affinity) manner with, another chemical entity specie (uncharged or charged atom or molecule) (in FIG. 1, schematically represented by hexagonal d2 and hexagonal d4, respectively) of chemical entity (X). Such metal surface (M)-chelator (C)-chemical entity (X) chelate type of coordination compound configurations can be symbolized as m4-c3-X (wherein X=L1-d2), and m8-c5-X (wherein X=L2-d4), respectively.

For each metal surface (M)-chelator (C)-chemical entity (X) chelate type of coordination compound configuration, m4-c3-X (wherein X=L1-d2), and m8-c5-X (wherein X=L2-d4), the bonding, or the at least bonding-like (affinity) interaction, between the corresponding (chelator bonded or interacting) chemical entity specie L1 and L2, respectively, and the corresponding additional chemical entity specie d2 and d4, respectively, of chemical entity (X), is of any type and number. The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof. For each metal surface (M)-chelator (C)-chemical entity (X) chelate type of coordination compound configuration, m4-c3-L1-d2, and m8-c5-L2-d4, such bonding, or at least bonding-like (affinity) interaction, is generally represented by a single (sideless) ladder of dashes extending between the (chelator bonded or interacting) chemical entity specie L1 and L2, and the corresponding additional chemical entity specie d2 and d4, respectively, of chemical entity (X).

There also exists a sub-population of (chelator bonded or interacting) chemical entity species, d1 and d3, each of which is not bonded to any other chemical entity specie of chemical entity (X). Within the scope of the present invention, in a non-limiting manner, it is to be fully understood that for a different given set of parameters of previous and current physicochemical treatments or/and conditions, metal surface (M) of medical implant component 12 of ‘metal chelated surface’ medical device 10, can include any number of different types of sub-populations and configurations of chelated (complexed) chelator molecules of chelator (C) which are bonded or non-bonded to chemical entity species of chemical entity (X), or/and include any number of different types of sub-populations and configurations of chemical entity species of chemical entity (X) which are bonded to chelated (complexed) chelator molecules of chelator (C), or/and which are bonded to other chemical entity species of chemical entity (X).

Accordingly, as just illustratively described with reference to FIG. 1, ‘metal chelated surface’ medical device 10 includes a metal surface (M) of a medical implant component 12 to which is bound a chemical entity (X) via a chelator (C) chelated to the metal surface (M) in an (M)-(C)-(X) chelate type of coordination compound configuration. Thus, in accordance with the previously stated main aspect of the present invention, there is provision of an implantable medical device characterized by including a medical implant component having a metal surface (M) to which is bound a chemical entity (X) via a chelator (C) chelated to the metal surface in an (M)-(C)-(X) configuration.

Additionally, with reference to FIG. 1, since ‘metal chelated surface’ medical device 10 features a metal surface (M) of a medical implant component 12 to which is bound a chemical entity (X) via a chelator (C) chelated to the metal surface (M) in an (M)-(C)-(X) chelate type of coordination compound configuration, then, by deduction, medical implant component 12 of ‘metal chelated surface’ medical device 10 can also be characterized by including, as a sub-combination, a chelate type of coordination compound characterized by having a structure of general formula: (C)-(X), wherein (C) is a chelator and (X) is a chemical entity chelated to the chelator in a chelate type of coordination compound configuration.

Each chelate type of coordination compound characterized by having a structure of general formula: (C)-(X), wherein (C) is a chelator and (X) is a chemical entity chelated to the chelator in a chelate type of coordination compound configuration, includes a chelator molecule, for example, c2, c3, c4, or c5, of chelator (C), which is chelated (complexed) to a chemical entity specie (uncharged or charged molecule), for example, d1, L1, d3, or L2, respectively, of chemical entity (X). Such chelator (C)-chemical entity (X) chelate type of coordination compounds can be symbolized as c2-X (wherein X=d1), c3-X (wherein X=L1), c4-X (wherein X=d3), and c5-X (wherein X=L2), respectively.

Each chelator (C)-chemical entity (X) chelate type of coordination compound configuration, c2-d1, c3-L1, c4-d3, and c5-L2, is characterized by having at least two coordinate covalent bonds between at least two coordinating groups of the corresponding chelator molecule c2, c3, c4, and c5, respectively, of chelator (C), and the corresponding chelated (complexed) chemical entity molecule d1, L1, d3, and L2, respectively, of chemical entity (X). For each chelate type of coordination compound configuration, c2-d1, c3-L1, c4-d3, and c5-L2, the at least two coordinate covalent bonds are generally represented by the single (sideless) ladder of dashes extending between each chelator molecule c2, c3, c4, and c5, of chelator (C), and the corresponding chelated (complexed) chemical entity molecule d1, L1, d3, and L2, respectively, of chemical entity (X).

In a similar manner as was previously illustratively described for the metal surface (M)-chelator (C)-chemical entity (X) chelate type of coordination compound configurations of medical implant component 12 of ‘metal chelated surface’ medical device 10, in the current or instant population of chelated (complexed) chemical entity species d1, L1, d3, and L2, of chemical entity (X), of chelator (C)-chemical entity (X) chelate type of coordination compound configurations, there exists a sub-population of chelated (complexed) chemical entity species, L1 and L2, each of which is additionally bonded to, or at least interacts in a bonding-like (affinity) manner with, another chemical entity specie (uncharged or charged atom or molecule), d2 and d4, respectively, of chemical entity (X). Such chelator (C)-chemical entity (X) chelate type of coordination compound configurations can be symbolized as c3-X (wherein X=L1-d2), and c5-X (wherein X=L2-d4), respectively.

For each chelator (C)-chemical entity (X) chelate type of coordination compound configuration, c3-X (wherein X=L1-d2), and c5-X (wherein X=L2-d4), the bonding, or the at least bonding-like (affinity) interaction, between the corresponding chelated (complexed) chemical entity molecule L1 and L2, respectively, of chemical entity (X), and the corresponding additional chemical entity specie d2 and d4, respectively, of chemical entity (X), is of any type and number. The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof. For each chelator (C)-chemical entity (X) chelate type of coordination compound configuration, c3-L1-d2, and c5-L2-d4, such bonding, or at least bonding-like (affinity) interaction, is generally represented by a single (sideless) ladder of dashes extending between the chelated (complexed) chemical entity molecule L1 and L2, and the corresponding additional chemical entity specie d2 and d4, respectively, of chemical entity (X).

A special case of the immediately preceding illustratively described chelator (C)-chemical entity (X) chelate type of coordination compounds of medical implant component 12 of ‘metal chelated surface’ medical device 10, is where chemical entity (X) is a chemical entity (Y) selected from the group consisting of (i) a drug (uncharged or charged molecule) or a biological moiety (uncharged or charged molecule), for example, chemical entity specie as drug or biological moiety molecule d1 or d3, chelated (complexed) to the chelator (C), for example, chelator molecule c2 or c4, respectively, and, (ii) a linker or spacer (uncharged or charged molecule), for example, chemical entity specie as linker molecule L1 or L2, having a first part chelated (complexed) to the chelator (C), for example, chelator molecule c3 or c5, respectively, and having a second part bonded to a drug (uncharged or charged atom or molecule) or a biological moiety (uncharged or charged atom or molecule), for example, chemical entity specie as drug molecule or biological moiety molecule d2 or d4, respectively. For sub-group (i), such chelator (C)-chemical entity (X) chelate type of coordination compound configurations can be symbolized as c2-X (wherein X=Y=d1), and c4-X (wherein X=Y=d3), respectively. For sub-group (ii), such chelator (C)-chemical entity (X) chelate type of coordination compound configurations can be symbolized as c3-X (wherein X=Y=L1-d2), and c5-X (wherein X=Y=L2-d4), respectively.

For sub-group (i) type of chelator (C)-chemical entity (X=Y=drug or biological moiety) chelate type of coordination compound configurations, each corresponding drug or biological moiety molecule d1 and d3, respectively, is chelated (complexed) to the corresponding chelator molecule c2 and c4, respectively, of chelator (C), in the form of a chelate type of coordination compound (metal complex, metal ion complex, coordination complex, chelate complex, chelate ring, or, chelate). Such specific embodiments of chelator (C)-chemical entity (X=Y=drug or biological moiety) chelate type of coordination compound configurations can be symbolized as c2-d1 and c4-d3, respectively.

Each chelator (C)-chemical entity (X=drug or biological moiety) chelate type of coordination compound configuration, c2-d1 and c4-d3, is characterized by having at least two coordinate covalent bonds between at least two coordinating groups of the corresponding chelator molecule c2 and c4, respectively, of chelator (C), and the corresponding chelated (complexed) drug or biological moiety molecule d1 and d3, respectively, of chemical entity (X=Y=drug). For each chelate type of coordination compound configuration, c2-d1 and c4-d3, the at least two coordinate covalent bonds are generally represented by a single (sideless) ladder of dashes extending between the corresponding chelator molecule c2 and c4 of chelator (C) and the corresponding chelated (complexed) drug or biological moiety molecule d1 and d3, respectively, of chemical entity (X=Y=drug or biological moiety).

For sub-group (ii) type of chelator (C)-chemical entity (X=Y=linker-drug, or linker-biological moiety) chelate type of coordination compound configurations, each corresponding linker molecule L1 and L2, respectively, has a first part chelated (complexed) to the corresponding chelator molecule c3 and c5, respectively, of the chelator (C), in the form of a coordination compound (metal complex, metal ion complex, coordination complex, chelate complex, chelate ring, or, chelate), and has a second part bonded to the corresponding drug or biological moiety molecule d2 and d4, respectively. Such specific embodiments of chelator (C)-chemical entity (X=Y=linker-drug, or linker-biological moiety) chelate type of coordination compound configurations can be symbolized as c3-L1-d2 and c5-L2-d4, respectively.

Each chelator (C)-chemical entity (X=Y=linker-drug, or linker-biological moiety) chelate type of coordination compound configuration, c3-L1-d2 and c5-L2-d4, is characterized by having at least two coordinate covalent bonds between at least two coordinating groups of the corresponding chelator molecule c3 and c5, respectively, of chelator (C), and the first part of the corresponding chelated (complexed) linker molecule L1 and L2, respectively, of chemical entity (X=Y=linker-drug, or linker-biological moiety). The at least two coordinate covalent bonds are generally represented by a single (sideless) ladder of dashes extending between the chelator molecule c3 and c5 of chelator (C), and the corresponding chelated (complexed) linker molecule L1 and L2, respectively, of chemical entity (X=Y=linker-drug, or linker-biological moiety).

Additionally, each chelator (C)-chemical entity (X=Y=linker-drug, or linker-biological moiety) chelate type of coordination compound configuration, c3-L1-d2 and c5-L2-d4, is further characterized by having at least one bond between the second part of the corresponding chelated (complexed) linker molecule L1 and L2, respectively, and the corresponding drug or biological moiety molecule d2 and d4, respectively, of chemical entity (X=Y=linker-drug, or linker-biological moiety). The bonding between the second part of the corresponding chelated (complexed) linker molecule L1 and L2, respectively, and the corresponding drug or biological moiety molecule d2 and d4, respectively, of chemical entity (X=Y=linker-drug, or linker-biological moiety), is of any type and number. The bonding can be at least one covalent bond, at least one ionic bond, at least one hydrogen bond, at least one van der Waals bond, at least one coordinate covalent bond, or a combination thereof. Such bonding is generally represented by a single (sideless) ladder of dashes extending between the chelated (complexed) linker molecule L1 and L2, and the corresponding drug or biological moiety molecule d2 and d4, respectively, of chemical entity (X=Y=linker-drug, or linker-biological moiety).

Regarding notation, based on the immediately preceding illustrative description of this special case of the chelator (C)-chemical entity (X) chelate type of coordination compounds (wherein chemical entity (X) is a chemical entity (Y)), then, it is fully understood that a chelator (C)-chemical entity (X) chelate type of coordination compound can be equivalently referred to as a chelator (C)-chemical entity (Y) chelate type of coordination compound, along with the full understanding that the just described structure and function of the components of the chelator (C)-chemical entity (X) chelate type of coordination compounds are equivalently applicable for describing structure and function of the components of the chelator (C)-chemical entity (Y) chelate type of coordination compounds.

Accordingly, as just illustratively described with reference to FIG. 1, the chelator (C)-chemical entity (X) chelate type of coordination compounds of medical implant component 12 of ‘metal chelated surface’ medical device 10, can be characterized by having a specific configuration or embodiment wherein chemical entity (X) is a chemical entity (Y) selected from the group consisting of (i) a drug chelated to the chelator or a biological moiety chelated to the chelator (C), and, (ii) a linker having a first part chelated to the chelator (C) and having a second part bonded to a drug or a biological moiety. Thus, in accordance with another main aspect of the present invention, the present invention also includes, as a sub-combination, provision of a chelate type of coordination compound characterized by having a structure of general formula: (C)-(Y), wherein (C) is a chelator and (Y) is a chemical entity selected from the group consisting of (i) a drug chelated to the chelator or a biological moiety chelated to the chelator, and, (ii) a linker having a first part chelated to the chelator and having a second part bonded to a drug or a biological moiety.

Charged States and Bonding Configurations of Metal Surface (M), Chelator (C), and Chemical Entity (X)

Following are additional details of the physicochemical properties, characteristics, and behavior, regarding the charged states and bonding configurations of metal surface (M), chelator (C), and chemical entity (X), singly, in combination, and in sub-combination, of the exemplary embodiment of ‘metal chelated surface’ medical device 10, of the present invention.

Regarding the specific electronic (charged (cationic or anionic), uncharged (neutral), or polarized) state of exposed surface metal ions and atoms m1-m11 of metal surface (M), for the exemplary embodiment or configuration of metal surface (M) of medical implant component 12 illustrated in FIG. 1, according to given parameters of previous and current physicochemical treatments or/and conditions, there exists a sub-population of exposed surface metal ions and atoms m1, m2, m4, m5, m7, m8, m10, and m11, of metal surface (M), each of which is charged (cationic or anionic), uncharged (neutral), or polarized, and is either chelated (complexed) (m1, m2, m4, m7, m8, and m10) or not chelated (m5 and m11) to a chelator molecule, for example, c1-c6, of chelator (C).

Within the sub-population of exposed surface metal ions and atoms m1, m2, m4, m5, m7, m8, m10, and m11, of metal surface (M), each chelated (complexed) exposed surface metal ion or atom m1, m2, m4, m7, m8, and m10, which is chelated (complexed) to a corresponding chelator molecule c1, c2, c3, c4, c5, and c6, respectively, of chelator (C), in the form of a previously illustratively described metal surface (M)-chelator (C) chelate type of coordination compound configurations, m1-c1, m2-c2, m4-c3, m7-c4, m8-c5, and m10-c6, respectively, was previously made charged (cationic or anionic) as a result of previously having been subjected to a metal surface activation procedure (for example, oxidation or reduction), as a necessary metal surface preparatory step prior to participating in the metal surface chelation reaction for forming the coordination compounds or chelates. Any chelated (complexed) exposed surface metal atom which is uncharged or only polarized, from within the group of all the chelated (complexed) exposed surface metal ions and atoms m1, m2, m4, m7, m8, and m10, became uncharged or only polarized as a result of participating in the metal surface chelation reaction for forming a coordination compound or chelate.

Additionally, within the sub-population of exposed surface metal ions and atoms m1, m2, m4, m5, m7, m8, m10, and m11, of metal surface (M), each non-chelated (non-complexed) exposed surface metal ion m5 and m11 is charged (cationic or anionic), as a result of previously being subjected to the above stated metal surface activation procedure (oxidation or reduction), but, for one or more reasons, did not participate in the metal surface chelation reaction for forming a coordination compound or chelate.

Additionally, in the current or instant population of exposed surface metal ions and atoms m1-m11, of metal surface (M), there exists a sub-population of non-chelated (non-complexed) exposed surface metal atoms m3, m6, and m9, each of which is uncharged (neutral) or at most polarized and not chelated (complexed) to any chelator molecule, for example, c1-c6, of chelator (C), apparently as a result of not participating in the metal surface activation procedure, and consequently, of not participating in the metal surface chelation reaction for forming a coordination compound or chelate.

In metal surface (M) of medical implant component 12 of ‘metal chelated surface’ medical device 10, illustrated in FIG. 1, each chelated (complexed) exposed surface metal ion or atom m1, m2, m4, m7, m8, and m10, which is chelated (complexed) to a corresponding chelator molecule c1, c2, c3, c4, c5, and c6, respectively, of chelator (C), in the form of a metal surface (M)-chelator (C) chelate type of coordination compound configuration, m1-c1, m2-c2, m4-c3, m7-c4, m8-c5, and m10-c6, respectively, has a positive, zero, or negative, valued oxidation state, with a positive valued oxidation state being most common, a zero valued oxidation state less common, and a negative valued oxidation state possible, and being least common and rare. Each chelator molecule c1, c2, c3, c4, c5, and c6, of chelator (C), involved in the formation of the corresponding metal surface (M)-chelator (C) chelate type of coordination compound configuration, m1-c1, m2-c2, m4-c3, m7-c4, m8-c5, and m10-c6, respectively, has a negative charge (anionic), a zero charge (neutral), or a positive charge (cationic), with a negative charge being most common, a zero charge less common, and a positive charge possible, and being least common and rare. Each metal surface (M)-chelator (C) chelate type of coordination compound configuration, m1-c1, m2-c2, m4-c3, m7-c4, m8-c5, and m10-c6, formed between each corresponding exposed surface metal ion or atom m1, m2, m4, m7, m8, and m10, respectively, of metal surface (M), and the corresponding chelator molecule c1, c2, c3, c4, c5, and c6, of chelator (C), has a combined or total zero (neutral), positive, or negative, net charge.

In metal surface (M) of medical implant component 12 of ‘metal chelated surface’ medical device 10, illustrated in FIG. 1, each chelated (complexed) exposed surface metal ion or atom m1, m2, m4, m7, m8, and m10, which is chelated (complexed) to a corresponding chelator molecule c1, c2, c3, c4, c5, and c6, respectively, of chelator (C), in the form of a metal surface (M)-chelator (C) chelate type of coordination compound configuration, m1-c1, m2-c2, m4-c3, m7-c4, m8-c5, and m10-c6, respectively, has a definite number of coordinating groups of the corresponding chelator molecule c1, c2, c3, c4, c5, and c6, respectively, of chelator (C), that it can accommodate within its coordination sphere in the eventually formed coordination compound or chelate. Accordingly, the ‘coordination number’ of each chelated (complexed) exposed surface metal ion or atom m1, m2, m4, m7, m8, and m10, of metal surface (M), is the number of (coordinate covalent) bonds formed by each chelated (complexed) exposed surface metal ion or atom m1, m2, m4, m7, m8, and m10, with the electron donor or electron acceptor coordinating groups of the corresponding chelator molecule c, c2, c3, c4, c5, and c6, respectively, of chelator (C).

The coordination number of each chelated (complexed) exposed surface metal ion or atom m1, m2, m4, m7, m8, and m10, of metal surface (M), is typically six or four. Lower and higher coordination numbers, for example, three and eight, respectively, of each metal ion or atom are possible. For example, as an analogy, with respect to coordination between a metal ion or atom and a plurality of individual (non-chelator) unidentate (one-toothed) ligands (complexing agents), involving formation of a plurality of coordinate covalent bonds with the individual ligands in the resulting (non-chelate) coordination compound, coordination number of the metal ion or atom in the range of between two and twelve is known, with a coordination number of six, four, and eight, in this order, being the most common.

Within the scope of the present invention, in a non-limiting manner, it is to be fully understood that for a ‘different’ given set of parameters of previous and current physicochemical treatments or/and conditions, in addition to the characteristic features of exemplary preferred embodiments of the ‘metal chelated surface’ medical device of the present invention illustratively described hereinabove with reference to FIG. 1, there are many numerous additional exemplary preferred embodiments, alternative preferred embodiments, specific configurations, and, additional and optional aspects, characteristics, or features, thereof, of the present invention, which may also be provided separately or in any suitable sub-combination. In particular, regarding ‘metal chelated surface’ medical device 10 including a medical implant component 12 having a metal surface (M) to which is chelated a chelator (C) in an (M)-(C) chelate type of coordination compound configuration. In particular, regarding ‘metal chelated surface’ medical device 10 including a metal surface (M) of a medical implant component 12 to which is bound a chemical entity (X) via a chelator (C) chelated to the metal surface (M) in an (M)-(C)-(X) chelate type of coordination compound configuration. In particular, regarding chelator (C)-chemical entity (X) chelate type of coordination compound configurations being characterized by having a specific configuration or embodiment wherein chemical entity (X) is a chemical entity (Y) selected from the group consisting of (i) a drug chelated to the chelator (C) or a biological moiety chelated to the chelator (C), and, (ii) a linker having a first part chelated to the chelator (C) and having a second part bonded to a drug or a biological moiety.

A few selected examples of different general and specific embodiments and configurations of the present invention, provided in combinations or/and in suitable sub-combinations, are illustratively described hereinbelow.

For example, regarding the metal surface (M)-chelator (C) chelate type of coordination compound configurations, and the metal surface (M)-chelator (C)-chemical entity (X) chelate type of coordination compound configurations, of ‘metal chelated surface’ medical device 10 illustrated in FIG. 1, in general, any ‘two or more’ chelator molecules selected from among c1-c6 of chelator (C), where each chelator molecule contains at least two coordinating groups, can be chelated (complexed) to a ‘single’ exposed surface metal ion selected from among m1, m2, m4, m5, m7, m8, m10, and m11, of metal surface (M), in the form of a chelate type of coordination compound configuration. Such a chelate type of coordination compound configuration formed between the two or more chelator molecules and the single metal ion or atom has a combined or total zero (neutral), positive, or negative, net charge.

Alternatively, for example, in general, any ‘single’ chelator molecule selected from among c1-c6 of chelator (C), where the single chelator molecule contains at least two coordinating groups, can be chelated (complexed) to ‘two or more’ exposed surface metal ions selected from among m1, m2, m4, m7, m8, and m10, of metal surface (M), in the form of a chelate type of coordination compound configuration. Such a chelate type of coordination compound configuration formed between the single chelator molecule and the two or more metal ions or atoms has a combined or total zero (neutral), positive, or negative, net charge. Ordinarily, one or more chelator molecules is chelated (complexed) with a single metal ion, rather than a single chelator molecule being chelated (complexed) with more than one metal ion, in the form of a chelate type of coordination compound configuration.

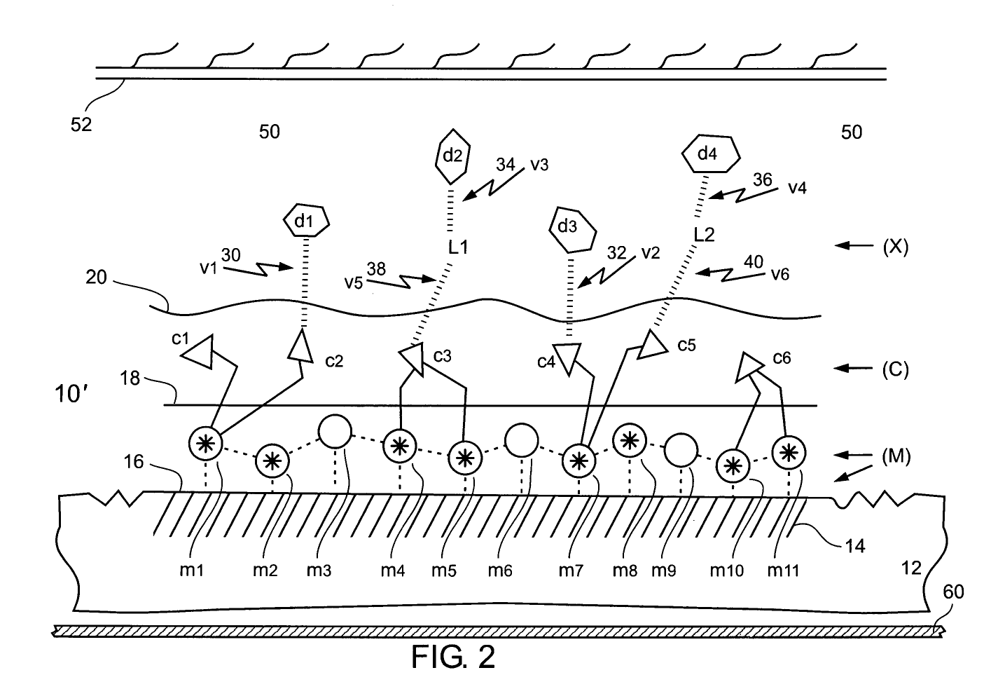

The above two specific examples are clearly illustrated in FIG. 2. ‘Metal chelated surface’ medical device 10′ illustrated in FIG. 2 is essentially the same as ‘metal chelated surface’ medical device 10 illustrated in FIG. 1, but includes different alternative types of configurations of coordinate covalent bonding between chelator molecules c1-c6 of chelator (C), and metal ions or atoms m1, m2, m4, m5, m7, m8, m10, and m11, of metal surface (M), in the chelate type of coordination compound configurations.

In FIG. 2, exemplary illustration of the immediately above first example is where two chelator molecules c1 and c2 of chelator (C) are both chelated (complexed) to a single exposed surface metal ion or atom m1 of metal surface (M), and where two chelator molecules c4 and c5 of chelator (C) are both chelated (complexed) to a single exposed surface metal ion or atom m7 of metal surface (M). Such metal surface (M)-chelator (C) chelate type of coordination compound configurations can be symbolized as c1-m1-c2, and c4-m7-c5, respectively. Exemplary illustration of the immediately above second example is where a single chelator molecule c3 of chelator (C) is chelated (complexed) to two exposed surface metal ions or atoms m4 and m5 of metal surface (M), and where a single chelator molecule c6 of chelator (C) is chelated (complexed) to two exposed surface metal ions or atoms m10 and m11 of metal surface (M). Such metal surface (M)-chelator (C) chelate type of coordination compound configurations can be symbolized as m4-c3-m5, and m10-c6-m11, respectively.

It is to be fully understood that, except for the different alternative types of configurations of coordinate covalent bonding between chelator molecules c1-c6 of chelator (C) and metal ions or atoms m1, m2, m4, m5, m7, m8, m10, and m11, of metal surface (M), in the chelate type of coordination compound configurations, all physicochemical structural and functional aspects, characteristics, and features, which were previously illustratively described for ‘metal chelated surface’ medical device 10 illustrated in FIG. 1, are clearly fully applicable for illustratively describing the same for ‘metal chelated surface’ medical device 10′ illustrated in FIG. 2.

Additionally, for example, regarding the metal surface (M)-chelator (C) chelate type of coordination compound configurations, and the metal surface (M)-chelator (C)-chemical entity (X) chelate type of coordination compound configurations, of ‘metal chelated surface’ medical device 10 and 10′ illustrated in FIGS. 1 and 2, respectively, in general, any ‘single’ metal chelated (complexed) chelator molecule, for example, selected from among c1-c6, of chelator (C), can be bonded to, or at least interact in a bonding-like (affinity) manner with, ‘two or more’ chemical entity species (uncharged or charged atoms or molecules), for example, selected from among d1, L1, d3, and L2, of chemical entity (X). Alternatively, for example, in general, any ‘two or more’ metal chelated (complexed) chelator molecules, for example, selected from among c1-c6, of chelator (C), can be bonded to, or at least interact in a bonding-like (affinity) manner, with a ‘single’ chemical entity specie (uncharged or charged atom or molecule), for example, selected from among d1, L1, d3, and L2, of chemical entity (X).

Additionally, for example, regarding the (sub-combination) chelator (C)-chemical entity (X) chelate type of coordination compound configurations of ‘metal chelated surface’ medical device 10 and 10′ illustrated in FIGS. 1 and 2, respectively, in general, any ‘single’ chelator molecule, for example, selected from among c1-c6, of chelator (C), can be chelated (complexed) to ‘two or more’ chemical entity species (uncharged or charged molecules), for example, selected from among d1, L1, d3, and L2, of chemical entity (X), in the form of a chelate type of coordination compound configuration. Alternatively, for example, in general, any ‘two or more’ chelator molecules, for example, selected from among c1-c6, of chelator (C), can be chelated (complexed) to a ‘single’ chemical entity specie (uncharged or charged molecule), for example, selected from among d1, L1, d3, and L2, of chemical entity (X), in the form of a chelate type of coordination compound configuration.

Additionally, for example, regarding the (sub-combination) chelator (C)-chemical entity (X) chelate type of coordination compound configurations of ‘metal chelated surface’ medical device 10 and 10′ illustrated in FIGS. 1 and 2, respectively, in general, a ‘single’ chelated (complexed) chemical entity specie (uncharged or charged molecule), for example, selected from among L1 and L2, of chemical entity (X), which is chelated (complexed) to at least one chelator molecule, for example, selected from among c1-c6, of chelator (C), can be additionally bonded to, or at least interact in a bonding-like (affinity) manner with, ‘two or more’ other chemical entity species (uncharged or charged atoms or/and molecules), for example, selected from among d2 and d4, of chemical entity (X).

Alternatively, for example, in general, any ‘two or more’ chelated (complexed) chemical entity species (uncharged or charged molecules), for example, selected from among L1 and L2, of chemical entity (X), which are chelated (complexed) to a ‘single’ chelator molecule, for example, selected from among c1-c6, of chelator (C), can be additionally bonded to, or at least interact in a bonding-like (affinity) manner with, another ‘single’ chemical entity specie (uncharged or charged atom or molecule), for example, selected from among d2 and d4, of chemical entity (X).

Stability, and Selective Cleavage or Breakage, of the Bonding or Bonding-Like (Affinity) Interaction in Metal Surface (M)-Chelator (C)-Chemical Entity (X) Configurations

Following are details regarding stability, and selective cleavage or breakage, of the various different types of bonding or bonding-like (affinity) interaction in the metal surface (M)-chelator (C)-chemical entity (X) chelate type of coordination compound configurations, and in the (sub-combination) chelator (C)-chemical entity (X) chelate type of coordination compound configurations, of ‘metal chelated surface’ medical device 10 and 10′ illustrated in FIGS. 1 and 2, respectively.

For each metal surface (M)-chelator (C)-chemical entity (X) chelate type of coordination compound configuration, m2-c2-X (wherein X=d1), m4-c3-X (wherein X=L1), m7-c4-X (wherein X=d3), and m8-c5-X (wherein X=L2), the bonding (at least one covalent bond, at least one ionic bond, at least one hydrogen bond, at least one van der-Waals bond, at least one coordinate covalent bond, or a combination thereof), or the at least bonding-like (affinity) interaction (dipole-dipole type, hydrophilic type, hydrophobic type, or a combination thereof), between the corresponding metal chelated (complexed) chelator molecule c2, c3, c4, and c5, respectively, of chelator (C), and the corresponding chemical entity specie d1, L1, d3, and L2, respectively, of chemical entity (X) (generally represented by the single (sideless) ladder of dashes extending between the corresponding metal chelated (complexed) chelator molecule c2, c3, c4, and c5, respectively, of chelator (C), and the corresponding chemical entity specie d1, L1, d3, and L2, respectively, of chemical entity (X)) is either stable (that is, not ordinarily cleavable or breakable), or is selectively cleavable or breakable via an appropriate bond or bond-like cleaving or breaking mechanism (for example, enzymatic reaction, or chemical reaction) and an appropriately corresponding bond or bond-like cleaving or breaking agent (for example, an enzyme, or other chemical type bond or bond-like cleaving or breaking agent).

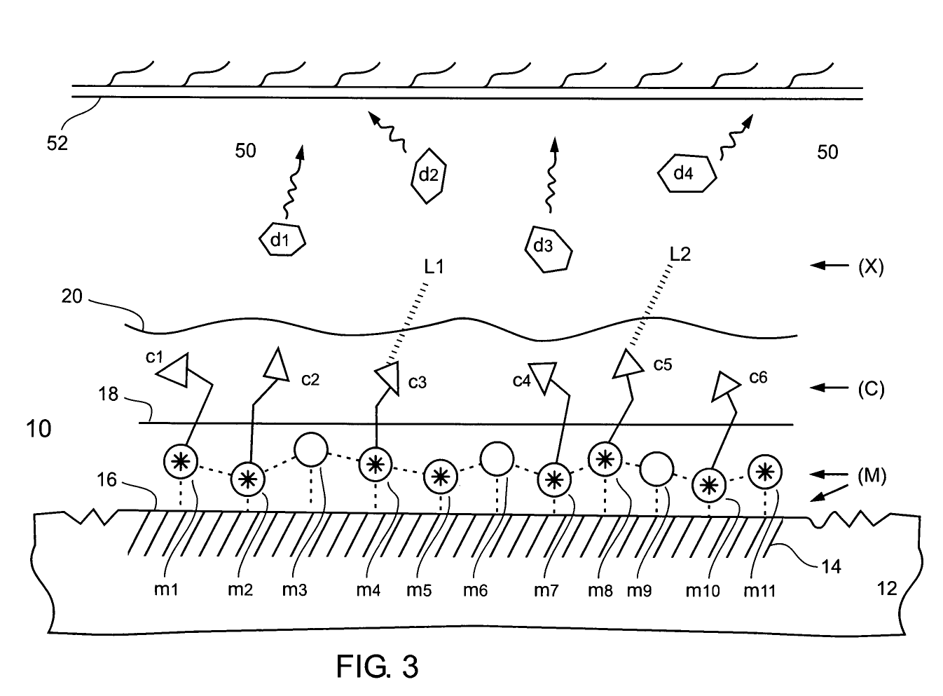

For example, in each metal surface (M)-chelator (C)-chemical entity (X) chelate type of coordination compound configuration, m2-c2-d1 and m7-c4-d3, the bonding, or the at least bonding-like (affinity) interaction, between the corresponding metal chelated (complexed) chelator molecule c2 and c4, respectively, of chelator (C), and the corresponding chemical entity specie d1 and d3, respectively, of chemical entity (X), is either stable (not ordinarily cleavable or breakable), or is selectively cleavable or breakable via bond or bond-like cleaving or breaking mechanism (curved arrows) 30 and 32, respectively, and an appropriately corresponding bond or bond-like cleaving or breaking agent v1 and v2, respectively, as illustrated in FIGS. 1 and 2. Such bond or bond-like cleavage or breakage results in separation, release or elution, and subsequent migration, of the corresponding chemical entity specie d1 and d3, respectively, of chemical entity (X), away from the corresponding metal chelated (complexed) chelator molecule c2 and c4, respectively, of chelator (C), as illustrated in FIG. 3, and further described hereinbelow.

For each metal surface (M)-chelator (C)-chemical entity (X) chelate type of coordination compound configuration, m4-c3-X (wherein X=L1-d2), and m8-c5-X (wherein X=L2-d4), the bonding, or the at least bonding-like (affinity) interaction, between the corresponding (chelator bonded or interacting) chemical entity specie L1 and L2, respectively, and the corresponding additional chemical entity specie d2 and d4, respectively, of chemical entity (X) (generally represented by a single (sideless) ladder of dashes extending between the (chelator bonded or interacting) chemical entity specie L1 and L2, and the corresponding additional chemical entity specie d2 and d4, respectively, of chemical entity (X)) is either stable (not ordinarily cleavable or breakable), or is selectively cleavable or breakable via an appropriate bond or bond-like cleaving or breaking mechanism (for example, enzymatic reaction, or chemical reaction) and an appropriately corresponding bond or bond-like cleaving or breaking agent (for example, an enzyme, or other chemical type bond or bond-like cleaving or breaking agent).

For example, in each metal surface (M)-chelator (C)-chemical entity (X) chelate type of coordination compound configuration, m4-c3-L1-d2, and m8-c5-L2-d4, the bonding, or the at least bonding-like (affinity) interaction, between the corresponding (chelator bonded or interacting) chemical entity specie L1 and L2, respectively, and the corresponding additional chemical entity specie d2 and d4, respectively, of chemical entity (X), is either stable (not ordinarily cleavable or breakable), or is selectively cleavable or breakable via bond or bond-like cleaving or breaking mechanism (curved arrows) 34 and 36, respectively, and an appropriately corresponding bond or bond-like cleaving or breaking agent v3 and v4, respectively, as illustrated in FIGS. 1 and 2. Such bond or bond-like cleavage or breakage results in separation, release or elution, and subsequent migration, of the corresponding additional chemical entity specie d2 and d4, respectively, away from the corresponding (chelator bonded or interacting) chemical entity specie L1 and L2, respectively, of chemical entity (X), as illustrated in FIG. 3, and further described hereinbelow.

Alternatively, for example, in each metal surface (M)-chelator (C)-chemical entity (X) chelate type of coordination compound configuration, m4-c3-L1-d2, and m8-c5-L2-d4, the bonding, or the at least bonding-like (affinity) interaction, between the corresponding metal chelated (complexed) chelator molecule c3 and c5, respectively, of chelator (C), and the corresponding chemical entity specie L1 and L2, respectively, of chemical entity (X), is either stable (not ordinarily cleavable or breakable), or is selectively cleavable or breakable via bond or bond-like cleaving or breaking mechanism (curved arrows) 38 and 40, respectively, and an appropriately corresponding bond or bond-like cleaving or breaking agent v5 and v6, respectively, as illustrated in FIGS. 1 and 2. Such bond or bond-like cleavage or breakage results in separation, release or elution, and subsequent migration, of the corresponding chemical entity specie L1-d2 and L2-d4, respectively, of chemical entity (X), away from the corresponding metal chelated (complexed) chelator molecule c3 and c5, respectively, of chelator (C).

In a similar manner, for each chelator (C)-chemical entity (X) chelate type of coordination compound configuration, c2-X (wherein X=d1), c3-X (wherein X=L1), c4-X (wherein X=d3), and c5-X (wherein X=L2), the bonding, or the at least bonding-like (affinity) interaction, between the corresponding chelator molecule c2, c3, c4, and c5, respectively, of chelator (C), and the corresponding chemical entity specie d1, L1, d3, and L2, respectively, of chemical entity (X), is either stable (not ordinarily cleavable or breakable), or is selectively cleavable or breakable via an appropriate bond or bond-like cleaving or breaking mechanism (for example, enzymatic reaction, or chemical reaction) and an appropriately corresponding bond or bond-like cleaving or breaking agent (for example, an enzyme, or other chemical type bond or bond-like cleaving or breaking agent).

For example, in each chelator (C)-chemical entity (X) chelate type of coordination compound configuration, c2-d1 and c4-d3, the bonding, or the at least bonding-like (affinity) interaction, between the corresponding chelator molecule c2 and c4, respectively, of chelator (C), and the corresponding chemical entity specie d1 and d3, respectively, of chemical entity (X), is either stable (not ordinarily cleavable or breakable), or is selectively cleavable or breakable via bond or bond-like cleaving or breaking mechanism 30 and 32, respectively, and an appropriately corresponding bond or bond-like cleaving or breaking agent v1 and v2, respectively, as illustrated in FIGS. 1 and 2. Such bond or bond-like cleavage or breakage results in separation, release or elution, and subsequent migration, of the corresponding chemical entity specie d1 and d3, respectively, of chemical entity (X), away from the corresponding chelator molecule c2 and c4, respectively, of chelator (C), as illustrated in FIG. 3, and further described hereinbelow.

In a similar manner, for each chelator (C)-chemical entity (X) chelate type of coordination compound configuration, c3-X (wherein X=L1-d2), and c5-X (wherein X=L2-d4), the bonding, or the at least bonding-like (affinity) interaction, between the corresponding (chelator bonded or interacting) chemical entity specie L1 and L2, respectively, and the corresponding additional chemical entity specie d2 and d4, respectively, of chemical entity (X), is either stable (not ordinarily cleavable or breakable), or is selectively cleavable or breakable via an appropriate bond or bond-like cleaving or breaking mechanism (for example, enzymatic reaction, or chemical reaction) and an appropriately corresponding bond or bond-like cleaving or breaking agent (for example, an enzyme, or other chemical type bond or bond-like cleaving or breaking agent).

For example, in each chelator (C)-chemical entity (X) chelate type of coordination compound configuration, c3-L1-d2, and c5-L2-d4, the bonding, or the at least bonding-like (affinity) interaction, between the corresponding (chelator bonded or interacting) chemical entity specie L1 and L2, respectively, and the corresponding additional chemical entity specie d2 and d4, respectively, of chemical entity (X), is either stable (not ordinarily cleavable or breakable), or is selectively cleavable or breakable via bond or bond-like cleaving or breaking mechanism 34 and 36, respectively, and an appropriately corresponding bond or bond-like cleaving or breaking agent v3 and v4, respectively, as illustrated in FIGS. 1 and 2. Such bond or bond-like cleavage or breakage results in separation, release or elution, and subsequent migration, of the corresponding additional chemical entity specie d2 and d4, respectively, away from the corresponding (chelator bonded or interacting) chemical entity specie L1 and L2, respectively, of chemical entity (X), as illustrated in FIG. 3, and further described hereinbelow.

In a similar manner, alternatively, for example, in each chelator (C)-chemical entity (X) chelate type of coordination compound configuration, c3-L1-d2, and c5-L2-d4, the bonding, or the at least bonding-like (affinity) interaction, between the corresponding chelator molecule c3 and c5, respectively, of chelator (C), and the corresponding chemical entity specie L1 and L2, respectively, of chemical entity (X), is either stable (not ordinarily cleavable or breakable), or is selectively cleavable or breakable via bond or bond-like cleaving or breaking mechanism 38 and 40, respectively, and an appropriately corresponding bond or bond-like cleaving or breaking agent v5 and v6, respectively, as illustrated in FIGS. 1 and 2. Such bond or bond-like cleavage or breakage results in separation, release or elution, and subsequent migration, of the corresponding chemical entity specie L1-d2 and L2-d4, respectively, of chemical entity (X), away from the corresponding chelator molecule c3 and c5, respectively, of chelator (C).

Within the scope of the present invention, in a non-limiting manner, it is to be fully understood that many additional general and specific embodiments and configurations of the present invention, regarding stability and selective cleavage or breakage of the various different types of bonding or bonding-like (affinity) interaction, in the metal surface (M)-chelator (C)-chemical entity (X) chelate type of coordination compound configurations, and in the (sub-combination) chelator (C)-chemical entity (X) chelate type of coordination compound configurations, of ‘metal chelated surface’ medical device 10 and 10′ illustrated in FIGS. 1 and 2, respectively, are possible.

Selective binding, Via Chelation (Complexation), of ‘Free’ Metal Ions by Metal Chelated (Complexed) Chelator Molecules in Metal Surface (M)-Chelator (C)-Chemical Entity (X) Configurations

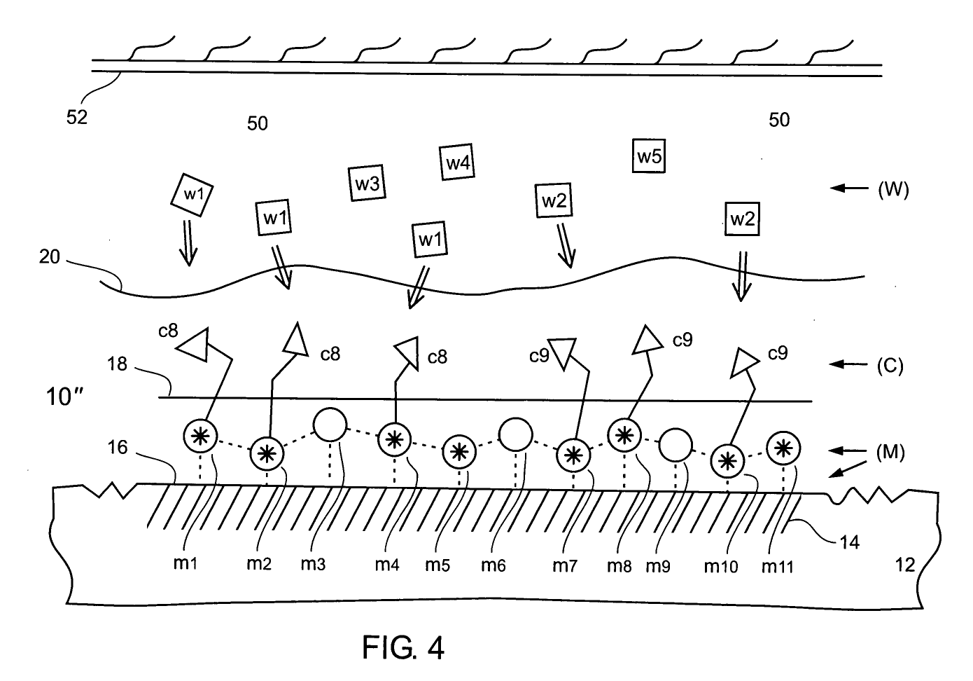

Following are details regarding selective binding, via chelation (complexation), of ‘free’ metal ions by metal chelated (complexed) chelator molecules in an exemplary ‘alternative’ preferred embodiment of the metal surface (M)-chelator (C) chelate type of coordination compound configurations of ‘metal chelated surface’ medical device 10 and 10′ illustrated in FIGS. 1 and 2, respectively, as illustrated in FIG. 4.

With reference to ‘metal chelated surface’ medical device 10″ illustrated in FIG. 4, for a given set of parameters of previous and current physicochemical treatments or/and conditions, in the current or instant population of exposed surface metal ions and atoms m1-m11, of metal surface (M), there exists a first sub-population of exposed surface metal ions and atoms m1, m2, and m4, each of which is charged (cationic or anionic), uncharged (neutral), or polarized, and is chelated (complexed) to a first type of chelator molecule c8 of chelator (C), in the form of a chelate type of coordination compound configuration, and there exists a second sub-population of exposed surface metal ions and atoms m7, m8, and m10, each of which is charged (cationic or anionic), uncharged (neutral), or polarized, and is chelated (complexed) to a second type of chelator molecule c9 of chelator (C), in the form of a chelate type of coordination compound configuration. Such metal surface (M)-chelator (C) chelate type of coordination compound configurations can be symbolized as m1-c8, m2-c8, m4-c8, and, m7-c9, m8-c9, m10-c9, respectively.

In each of the first and second sub-populations of metal surface (M)-chelator (C) chelate type of coordination compound configuration, m1-c8, m2-c8, m4-c8, and, m7-c9, m8-c9, m10-c9, respectively, the corresponding metal chelated (complexed) chelator molecule c8 and c9, respectively, of chelator (C), has the bonding potential (affinity) and capacity for selectively binding, via chelating (complexing), at least one ‘free’ metal ion, for example, free metal ions (squares) w1, w2, w3, w4, or/and w5, which originate not from metal surface (M) or from chemical entity (X), but rather, from a free metal ion source (W) which is totally separate from, and external to, ‘metal chelated surface’ medical device 10″, for potentially forming a metal surface (M)-chelator (C)-metal ion/atom (W) chelate type of coordination compound configuration. An exemplary free metal ion source (W) corresponds to free metal ions, for example, free metal ions (squares) w1, w2, w3, w4, or/and w5, which are mobile throughout a fluid, for example, blood, circulating through a cavity, for example, cavity 50, of a blood vessel having a blood vessel wall 52, as illustrated in FIG. 4.

As exemplified in FIG. 4, each metal chelated (complexed) chelator molecule c8 and c9 of chelator (C), is shown as having a preferred bonding potential (affinity) and capacity for selectively binding, via chelating (complexing), a single ‘free’ metal ion w1 or w2, respectively, from all free metal ions w1-w5, of free metal ion source (W), for potentially forming a metal surface (M)-chelator (C)-metal ion/atom (W) chelate type of coordination compound configuration. In FIG. 4, this preferred bonding potential (affinity) is schematically represented by a double-tailed arrow extending in the direction from each free metal ion w1 and w2, toward a corresponding metal chelated (complexed) chelate molecule c8 and c9, respectively. Such potentially formed metal surface (M)-chelator (C)-metal ion/atom (W) chelate type of coordination compound configurations can be symbolized as m1-c8-W, m2-c8-W, m4-c8-W, wherein W=w1, and, m7-c9-W, m8-c9-W, m10-c9-W, wherein W=w2, respectively.

Accordingly, each potentially formed metal surface (M)-chelator (C)-metal ion/atom (W) chelate type of coordination compound configuration, m1-c8-w1, m2-c8-w1, m4-c8-w1, and, m7-c9-w2, m8-c9-w2, m10-c9-w2, is firstly characterized by having at least two coordinate covalent bonds between the corresponding chelated (complexed) exposed surface metal ion or atom m1, m2, m4, and, m7, m8, m10, respectively, of metal surface (M), and at least two coordinating groups of the corresponding chelator molecule c8 and c9, respectively, of chelator (C), and is secondly characterized by having at least two coordinate covalent bonds between at least two coordinating groups of the corresponding metal chelated (complexed) chelator molecule c8 and c9, respectively, of chelator (C), and the corresponding chelated (complexed) metal ion (or atom) w1 and w2, respectively, previously being ‘free’ metal ions w1 and w2, respectively, from free metal ion source (W).

Within the scope of the present invention, in a non-limiting manner, it is to be fully understood that many additional general and specific embodiments and configurations of the present invention, regarding selective binding, via chelation (complexation), of ‘free’ metal ions by metal chelated (complexed) chelator molecules in the metal surface (M)-chelator (C) chelate type of coordination compound configurations of ‘metal chelated surface’ medical device 10″ illustrated in FIG. 4, are possible.

According to another main aspect of the present invention, there is provided an implantable medical device characterized by including a medical implant component having a surface to which is bound a chemical at a surface concentration of greater than 100 picograms (pg) per cm².

An important physicochemical property and characteristic of the present invention concerns the extent or amount of surface coverage by, and surface concentration of, the chemical or chemicals, chelator (C) or/and chemical entity (X), singly, in combination, or in sub-combination, in any of the hereinabove illustratively described metal surface (M)-chelator (C) chelate type of coordination compound configurations, or metal surface (M)-chelator (C)-chemical entity (X) chelate type of coordination compound configurations, of ‘metal chelated surface’ medical device 10, 10′, and 10″ (FIGS. 1-4), which is (are) bound on metal surface (M) of medical implant component 12 of ‘metal chelated surface’ medical device 10, 10′, or 10″, respectively. This extent or amount of surface coverage or surface concentration of a chemical or of chemicals bound to metal surface (M) corresponds to the bound chemical or chemicals being in the form of a coating on metal surface (M).

Accordingly, metal surface (M) is either partly, or entirely, coated by one or more chemicals which are bound on metal surface (M). Partial or entire coating, herein, also referred to as ‘surface coating’, of metal surface (M) of medical implant component 12 of ‘metal chelated surface’ medical device 10, 10′, or 10″ (FIGS. 1-4), is represented by the region, herein, also referred to as ‘surface coating region’, which extends from between interface layer or continuum 18 of exposed surface metal ions and atoms m1-m11 of metal surface (M) and surface bound metal chelated (complexed) chelator molecules c1-c6 of chelator (C), and above interface layer or continuum 20 of metal chelated (complexed) chelator molecules c1-c6 of chelator (C) and chemical entity species d1, L1, d3, and L2, of chemical entity (X). The extent or amount of surface coverage or surface concentration of the chemical or chemicals bound to metal surface (M) in the form of a surface coating in the surface coating region can be expressed in appropriate quantitative terms.

In general, the extent or amount of surface coverage or surface concentration of any of the above illustratively described various components of chelator (C) or/and chemical entity (X), singly, in combination, or in sub-combination, which is bound on metal surface (M) of medical implant component 12, in the form of a surface coating in the surface coating region, is defined in terms of appropriate mass (weight) and molar quantities, and ranges thereof, of the single component, of the combination of components, or/and of the sub-combination of a component, of chelator (C) or/and chemical entity (X), which is bound on metal surface (M), with respect to (per) an appropriate unit of surface area of metal surface (M) of medical implant component 12.

The appropriate mass (weight) and molar quantities, and ranges thereof, of any of the above illustratively described single component, combination of components, or/and sub-combination of a component, of chelator (C) or/and chemical entity (X), which is bound on metal surface (M), and the appropriate unit of surface area of metal surface (M) of medical implant component 12, are determined by that which is appropriate for describing, illustrating, and understanding, implementation of the relevant aspects and parameters relating to the extent or amount of surface coverage by, and surface concentration of, the single component, the combination of components, or/and the sub-combination of a component, of chelator (C) or/and chemical entity (X), which is bound on metal surface (M), within the field and scope of the present invention.

As previously stated above, the present invention relates to medical devices in the form of medical implants or medical implant components to which are bound chemicals, manufacturing thereof, and applications thereof, and more particularly, to a medical device featuring a medical implant or medical implant component having a metal surface to which is bound a chemical entity via a chelator chelated to the metal surface, manufacturing thereof, and applications thereof. An exemplary medical implant or medical implant component having a metal surface which is particularly suitable for applying the present invention is a stent. Chemical entities which are suitable for applying the present invention are essentially any of a wide variety of different categories and types of chemical compounds, for example, a drug or a biological moiety, a linker or spacer capable of binding a drug or a biological moiety, and a linker or spacer to which a drug or a biological moiety is bound. In an exemplary preferred embodiment of the present invention, the chelator is chelated to the metal surface of the medical implant or medical implant component in a form of a coating, whereupon the chemical entity (linker-drug) bound to the metal surface via the chelator coating results in the formation of a drug coated or drug eluting medical implant device, for example, a drug coated or drug eluting stent, wherein the bound chemical entity exhibits efficacy for preventing or/and treating a medical condition, disease, or ailment, such as restenosis, in general, in-stent restenosis, in particular, or/and thrombosis, in a human or animal subject.

As previously illustratively described hereinabove, medical implant component 12 generally corresponds to, and is generally representative of, at least a section of at least a part or component having a metal surface (M), of an entire or whole medical implant, such as a stent or a prosthesis. As an example, medical implant component 12 can generally correspond to, and be generally representative of, at least a section of at least a metal wire, a metal filament, or a metal thread, of a stent, or, alternatively, at least a section of a metal film, a metal plating, or a metal coating, deposited upon at least a section of another non-metal or metal part or component of a stent. As an example, medical implant component 12 can generally correspond to, and be generally representative of, at least a section of at least a metal plate, a metal joint, a metal fin, a metal screw, a metal spike, a metal wire, a metal filament, a metal thread, a metal anchor, or another metallic bone fixation element, of a prosthesis, or alternatively, at least a section of at least a metal film, a metal plating, or a metal coating, deposited upon at least a section of another non-metal or metal part or component of a prosthesis. Alternatively, medical implant component 12 may also generally correspond to, and be representative of, an entire or whole part or component having a metal surface (M), of a medical implant, such as a stent or a prosthesis, or, alternatively, an entire or whole medical implant having a metal surface (M), such as an entire or whole stent having a metal surface (M), or an entire or whole prosthesis having a metal surface (M).

Within the field and scope of the present invention, regarding the manufacture and use of implantable medical devices such as stents and prostheses, for defining the extent or amount of surface coverage or surface concentration, the appropriate mass and molar quantities, and ranges thereof, of any of the above illustratively described single component, combination of components, or/and sub-combination of a component, of chelator (C) or/and chemical entity (X), which is bound on an appropriate unit of surface area of metal surface (M) of medical implant component 12, in the surface coating region in the form of a surface coating, are of the order of magnitude of micrograms (μg) and micromoles (μmol), respectively, however, in general, implementation of the present invention can be performed wherein mass and molar quantities, and ranges thereof, are as low as picograms (pg) and picomoles (pmol), respectively.

Within the field and scope of the present invention, regarding the manufacture and use of implantable medical device such as stents and prostheses, for defining the extent or amount of surface coverage or surface concentration, at the micro (atomic, molecular, compound) level, appropriate units of surface area of metal surface (M) are, for example, a square Angstrom (Å²), a square nanometer (nm²), and a square micron (μm²). At the macro (coating) level, appropriate units of surface area of metal surface (M) are, for example, a square millimeter (mm²) and a square centimeter (cm²).

These orders of magnitude are based on the following empirical data regarding actual surface concentrations or dosage levels of drugs that have been used for attempting to prevent or/and inhibit onset or/and progression of restenosis, in general, and in-stent restenosis, in particular. As a first example, as previously cited hereinabove in the Background, in Gershlick, A., et al., 2004, a polymer-free based ‘paclitaxel’ drug eluting stent (V-Flex Plus coronary stent, Cook Inc.) was evaluated in Europe for safety and efficacy with respect to inhibition of in-stent restenosis. Escalating doses of paclitaxel (0.2, 0.7, 1.4, and 2.7 μg/mm² stent surface area) were directly applied, via a dipping procedure, to the abluminal surface of the stent, which was then implanted in the immediate vicinity of de novo lesions. As a second example, among the four main categories (anti-neoplastic (anti-inflammatory) drugs, immunosupressive (anti-proliferative) drugs, migration inhibitor (ECM modulator) drugs, and enhanced healing (re-endothelialization) drugs) of types of drugs that are currently used for attempting to prevent or/and inhibit onset or/and progression of restenosis, in general, and in-stent restenosis, in particular, and which are suitable for implementing the present invention, in the enhanced healing category are very potent growth factor drugs, such as VEGF (vascular endothelial growth factor) and FGF (fibroblast growth factor), where the working concentration in solution is in the range of between about 1 and 10 ng/ml (1000 and 10,000 pg/ml, respectively).

Thus, for implementing the present invention, for defining the extent or amount of surface coverage or surface concentration, appropriate mass and molar quantities, and ranges thereof, of any of the above illustratively described single component, combination of components, or/and sub-combination of a component, of chelator (C) or/and chemical entity (X), in any of the hereinabove illustratively described metal surface (M)-chelator (C) chelate type of coordination compound configurations, or metal surface (M)-chelator (C)-chemical entity (X) chelate type of coordination compound configurations, of ‘metal chelated surface’ medical device 10, 10′, or 10″ (FIGS. 1-4), which is bound on metal surface (M) in the form of a surface coating in the surface coating region, with respect to (per) an appropriate unit of surface area of metal surface (M) of medical implant component 12, are of the order of magnitude of micrograms (pg) and micromoles (pmol), respectively, of the single component, the combination of components, or/and the sub-combination of the component, per square millimeter (mm²) or per square centimeter (cm²) of metal surface (M). However, in general, implementation of the present invention can be performed wherein mass and molar quantities, and ranges thereof, are as low as picograms (pg) and picomoles (pmol), respectively, of the single component, the combination of components, or/and the sub-combination of the component, per square millimeter (mm²) or per square centimeter (cm²) of metal surface (M).

Thus, for implementing the present invention, for defining the extent or amount of surface coverage or surface concentration, the minimal or lower limit mass and molar quantities, and ranges thereof, of any of the above illustratively described single component, combination of components, or/and sub-combination of a component, of chelator (C) or/and chemical entity (X), in any of the hereinabove illustratively described metal surface (M)-chelator (C) chelate type of coordination compound configurations, or metal surface (M)-chelator (C)-chemical entity (X) chelate type of coordination compound configurations, of ‘metal chelated surface’ medical device 10, 10′, or 10″ (FIGS. 1-4), which is bound on metal surface (M), in the surface coating region in the form of a surface coating, with respect to (per) an appropriate unit of surface area of metal surface (M) of medical implant component 12, are greater than 100 picograms (pg) and greater than 1 picomole (pmol), respectively, of the single component, the combination of components, or/and the sub-combination of the component, per square centimeter (cm²) of metal surface (M).

Thus, in accordance with another main aspect of the present invention, there is provision of a medical device, in particular, ‘metal chelated surface’ medical device 10, 10′, or 10″ (FIGS. 1-4), characterized by including a medical implant component, in particular, medical implant component 12, having a surface, for example, a metal surface, in particular, metal surface (M), to which is bound a chemical, at a surface concentration of greater than 100 picograms (pg) per cm². The chemical can be any of the above illustratively described single component, combination of components, or/and sub-combination of a component, of chelator (C) or/and chemical entity (X), in any of the hereinabove illustratively described metal surface (M)-chelator (C) chelate type of coordination compound configurations, or metal surface (M)-chelator (C)-chemical entity (X) chelate type of coordination compound configurations, of ‘metal chelated surface’ medical device 10, 10′, or 10″ (FIGS. 1-4).

Additional Properties, Characteristics, and Aspects, of Metal Surface (M)

As previously illustratively described hereinabove, in a non-limiting manner, it is to be fully understood that in ‘metal chelated surface’ medical device 10, 10′, or 10″ (FIGS. 1-4), medical implant component 12 generally corresponds to, and is generally representative of, at least a section of at least a part or component having a metal surface (M), of an entire or whole medical implant, such as a stent or a prosthesis, or, alternatively, generally corresponds to, and is generally representative of, at least a section of a metal film, a metal plating, or a metal coating, deposited upon at least a section of another non-metal or metal part or component of an entire or whole medical implant, such as a stent or a prosthesis. Moreover, metal surface (M) represents an external (outer) side or/and an internal (inner) side of medical implant component 12.

For example, in the case that medical implant component 12 represents at least a section of a metal wire, a metal filament, or a metal thread, of a stent (for example, which is deliverable to, and implantable at, a pre-determined position in a subject, for example, inside the cavity of a blood vessel, for longitudinally extending along the side of the blood vessel wall), having an external (outer or abluminal) side (for example, facing the blood vessel wall), and an internal (inner or luminal) side (for example, facing the hollow inside or lumen of the stent), then metal surface (M) represents an external (outer or abluminal) side or/and an internal (inner or luminal) side of at least a section of the metal wire, metal filament, or metal thread, of the stent. For illustrative purposes only, in a non-limiting manner, as an example, as shown illustrated in FIG. 1, metal surface (M) of medical implant component 12 represents an external (outer or abluminal) side of at least a section of a metal wire, a metal filament, or a metal thread, of a stent (for example, which is deliverable to, and implantable at, a pre-determined position in a subject, for example, inside the cavity, for example, cavity 50, of a blood vessel and longitudinally extending along the side of the blood vessel wall, for example, blood vessel wall 52), having an external (outer or abluminal) side facing blood vessel wall 52.

Accordingly, for example, in the case that medical implant component 12 represents at least a section of a metal wire, a metal filament, or a metal thread, of a stent having an external (outer or abluminal) side facing a blood vessel wall and an internal (inner or luminal) side facing the hollow inside or lumen of the stent, then the above illustratively described and quantified extent or amount of surface coverage or surface concentration of a chemical or of chemicals bound to metal surface (M) corresponds to the bound chemical or chemicals being in the form of a surface coating on at least a section of a metal wire, a metal filament, or a metal thread, of an external (outer or abluminal) side or/and an internal (inner or luminal) side, of the section of the metal wire, metal filament, or metal thread, of the stent.

For illustrative purposes only, in a non-limiting manner, as an example, as shown illustrated in FIGS. 1-4, for metal surface (M) of medical implant component 12 representing an external (outer or abluminal) side of at least a section of a metal wire, a metal filament, or a metal thread, of a stent, facing a blood vessel wall, for example, blood vessel wall 52, then the above illustratively described and quantified extent or amount of surface coverage or surface concentration of a chemical or of chemicals bound to metal surface (M) corresponds to the bound chemical or chemicals being in the form of a surface coating on an external (outer or abluminal) side of at least a section of a metal wire, a metal filament, or a metal thread, of a stent, facing a blood vessel wall, for example, blood vessel wall 52.

Metal surface (M) of medical implant component 12 of ‘metal chelated surface’ medical device 10, 10′, or 10″ (FIGS. 1-4), is composed of a material which includes at least one metal element whose atoms can be ionized, in particular, by oxidation or reduction, such that a given ion (cation or anion) so formed on the surface, for example, exposed surface metal ion m1, m2, m4, m7, m8, or/and m10, of metal surface (M), is capable of being chelated (complexed) to at least two coordinating groups of at least a single chelator (chelating group, chelating agent, or complexing agent) molecule, for example, chelator molecule c1, c2, c3, c4, c5, c6, c8, or/and c9, of chelator (C), for forming a chelate type of coordination compound (metal complex, metal ion complex, coordination complex, chelate complex, chelate ring, or, chelate) configuration.

Metal surface (M) of medical implant component 12 is composed of a material selected from the group consisting of a metallic material, a semi-metallic material (metalloid), and a combination thereof. Such a material includes at least one metal element, at least one metal alloy each of two or more metal elements, or a combination thereof. In general, the metallic material or semi-metallic (metalloid) material includes at least one metal element selected from all the metal elements of the periodic table of elements, singly or in combination. Preferably, the at least one metal element is selected from the group consisting of transition metal elements, where the transition metal elements are scandium [Sc], titanium [Ti], vanadium [V], chromium [Cr], manganese [Mn], iron [Fe], cobalt [Co], nickel [Ni], copper [Cu], zinc [Zn], yttrium [Y], zirconium [Zr], niobium [Nb], molybdenum [Mo], technetium [Tc], ruthenium [Ru], rhodium [Rh], palladium [Pd], silver [Ag], cadmium [Cd], lutetium [Lu], hafnium [Hf], tantalum [Ta], tungsten [W], rhenium [Re], osmium [Os], iridium [Ir], platinum [Pt], and gold [Au].

Alternatively, or additionally, the at least one metal element includes at least one non-transition metal selected from the group consisting of beryllium [Be], aluminum [Al], indium [In], tin [Sn], and antimony [Sb]. An exemplary semi-metallic material (metalloid) is tellurium [Te].

Preferably, the at least one metal element which is/are suitable for being included in the metallic or semi-metallic material composing metal surface (M) of medical implant component 12 is/are is selected from those metal elements which are taught about and known in the art for being used as a metal surface of at least part of a medical device, in the form of a medical implant or medical implant component. In a non-limiting manner, such metal elements are selected from the group consisting of titanium [Ti], vanadium [V], chromium [Cr], iron [Fe], cobalt [Co], nickel [Ni], copper [Cu], zinc [Zn], niobium [Nb], molybdenum [Mo], rhodium [Rh], palladium [Pd], silver [Ag], tantalum [Ta], tungsten [W], rhenium [Re], osmium [Os], iridium [Ir], platinum [Pt], gold [Au], beryllium [Be], and aluminum [Al].

In a non-limiting manner, the at least one metal alloy which is/are suitable for being included in the metallic or semi-metallic material composing metal surface (M) of medical implant component 12 is/are selected from the group consisting of a shape memory alloy (SMA), a stainless steel alloy, a nickel-titanium [Ni—Ti] alloy (e.g., Nitinol™), a cobalt-molybdenum-chromium [Co—Mo—Cr] alloy, a beryllium-copper [Be—Cu] alloy, a cobalt-chromium [Co—Cr] alloy (e.g., Elgiloy™), a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] (TAV) alloy, and a titanium-aluminum-nickel [Ti—Al—Ni] (TAN) alloy.

Additional Properties, Characteristics, and Aspects, of Chelator (C)

Chelator (C), as part of the previously defined surface coating region in the form of a surface coating, is composed of any single type of molecule, or combination of two or more single types of molecules, for example, chelator molecules c1-c6, c8, c9, wherein each chelator molecule has at least two coordinating (complexing, chelating) groups in its structure and functions as a multi-dentate or multifunctional ligand for complexing or chelating to (coordinating with) at least a single metal ion (or atom) of a metal surface, such as metal surface (M) of medical implant component 12 of ‘metal chelated surface’ medical device 10, 10′, or 10″ (FIGS. 1-4), via at least two coordinate covalent bonds, for forming a chelate type of coordination compound (metal complex, metal ion complex, coordination complex, chelate complex, or chelate ring) configuration.

As previously described hereinabove, along with reference to FIGS. 1 and 2, preferably, each metal chelated (complexed) chelator molecule, for example, c2, c3, c4, and c5, of chelator (C), has the potential for bonding to, or at least interacting in a bonding-like (affinity) manner with, a chemical entity specie (uncharged or charged atom or molecule), for example, d1, L1, d3, and L2, respectively, of chemical entity (X). The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof. In an exemplary preferred embodiment of the present invention, the bonding or the at least bonding-like (affinity) interaction, is either stable (that is, not ordinarily cleavable or breakable), or is cleavable or breakable via an appropriate bond or bond-like cleaving or breaking mechanism (for example, enzymatic reaction or chemical reaction) and an appropriately corresponding bond or bond-like cleaving or breaking agent (for example, an enzyme, or other chemical type bond or bond-like cleaving or breaking agent), resulting in separation, release or elution, and subsequent migration, of the corresponding chemical entity specie d1, L1, d3, and L2, respectively, of chemical entity (X), away from the corresponding metal chelated (complexed) chelator molecule c2, c3, c4, and c5, respectively, of chelator (C), as previously illustratively described and exemplified with reference to FIGS. 1, 2, and 3.

As previously described hereinabove, along with reference to FIG. 4, in an exemplary preferred embodiment of the present invention, a metal chelated (complexed) chelator molecule, for example, c8 or c9, of chelator (C), has the bonding potential (affinity) and capacity for selectively binding, via chelating (complexing), one or more ‘free’ metal ions, for example, free metal ions w1, w2, w3, w4, or/and w5, which originate not from metal surface (M) or from chemical entity (X), but rather, from a free metal ion source (W) which is totally separate from, and external to, ‘metal chelated surface’ medical device 10″, for potentially forming a metal surface (M)-chelator (C)-metal ion/atom (W) chelate type of coordination compound configuration. Moreover, each such metal chelated (complexed) chelator molecule c8 and c9 of chelator (C), can have a preferred bonding potential (affinity) and capacity for selectively binding, via chelating (complexing), a single ‘free’ metal ion, for example, w1 or w2, respectively, from all free metal ions, for example, w1-w5, of the free metal ion source (W), for potentially forming a metal surface (M)-chelator (C)-metal ion/atom (W) chelate type of coordination compound configuration.

In a non-limiting manner, exemplary chelator (C) compounds, and molecules thereof, which are suitable for implementing the present invention, are selected from the group consisting of bifunctional acids, amino acids, peptides, proteins, ethylenediamine (en), propylenediamine (pn), diethylenetriamine (dien), triethylenetetraamine (trien), ethylenediaminetetraacetic acid (EDTA), ethyleneglycol bis(aminoethylether) tetraacetic acid (EGTA), hydroxyquinolates (for example, 8-hydroxyquinolate), hydroxyquinones, aminoquinones, phenanthroline, acetylacetone, oxalic acid, bifunctional acids such as citric acid, ascorbic acid, succinic acid; 4,5-dihydroxy-naphthalene disulfonic acid; N-nitrosophenylhydroxyamine ammonium salt; diantipyrylmethane, 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP); and combinations thereof.

For implementing the present invention, preferably, for a given specific material composition (as described hereinabove) of metal surface (M) of medical implant component 12 of ‘metal chelated surface’ medical device 10, 10′, or 10″ (FIGS. 1-4), preferably, there is identifying or/and testing, for example, by using standard prior art techniques, and subsequently using in the present invention, one or more specific types of chelator (C) compounds which are known, or are expected, to have structure and function as a multi-dentate or multifunctional ligand for complexing or chelating to (coordinating with) metal ions (or atoms) of that material composition of metal surface (M).

As a first example, for a material composition (as described hereinabove) including titanium in metal surface (M) of medical implant component 12, among the entire hereinabove list of chelator (C) compounds, each of the following: 4,5-dihydroxy-naphthalene disulfonic acid; N-nitrosophenylhydroxyamine ammonium salt;

diantipyrylmethane; 8-hydroxyquinoline; 2′,4′,5,7-tetrahydroxy-3,4-di-flavone; and 3,5-pyrocatecholdisulfonic acid, is known to have structure and function as a multi-dentate or multifunctional ligand for complexing or chelating to (coordinating with) titanium metal ions (or atoms) of the titanium containing material composition of metal surface (M).

Among these, diantipyrylmethane and 2′,4′,5,7-tetrahydroxy-3,4-di-flavone are particularly known and used as chemical reagents in a wide variety of applications for identifying titanium.

As a second example, for a material composition (as described hereinabove) including nickel in metal surface (M) of medical implant component 12, among the entire hereinabove list of chelator (C) compounds, each of the following: nitrilotriacetic acid (NTA); diethylenetriamine-penta-acetic acid (DTPA); quinoline-2-carboxylate; histidine (amino acid); and 6His (6 histidine peptide), is known to have structure and function as a multi-dentate or multifunctional ligand for complexing or chelating to (coordinating with) nickel metal ions (or atoms) of the nickel containing material composition of metal surface (M). An exemplary material composition including nickel in metal surface (M) of medical implant component 12, is nickel-titanium [Ni—Ti] alloy (e.g., Nitinol™).

As a third example, for a material composition (as described hereinabove) including copper in metal surface (M) of medical implant component 12, among the entire hereinabove list of chelator (C) compounds, each of the following: N-acetylcystein amide (amino acid); D-penicillamine; RGD (peptide); Cu/Zn superoxide dismutase (protein); Atoxl (protein); and hemoplexin (protein), is known to have structure and function as a multi-dentate or multifunctional ligand for complexing or chelating to (coordinating with) copper metal ions (or atoms) of the copper containing material composition of metal surface (M). An exemplary material composition including copper in metal surface (M) of medical implant component 12, is a copper-containing stainless steel alloy. Each of N-acetylcystein amide; D-penicillamine; and RGD-peptide, is particularly known to have structure and function as a multi-dentate or multifunctional ligand for complexing or chelating to (coordinating with) copper metal ions (or atoms) of a copper-containing stainless steel alloy material composition of metal surface (M).

As a fourth example, for a material composition (as described hereinabove) including cobalt in metal surface (M) of medical implant component 12, among the entire hereinabove list of chelator (C) compounds, quinoline-2-carboxylate, is known to have structure and function as a multi-dentate or multifunctional ligand for complexing or chelating to (coordinating with) cobalt metal ions (or atoms) of the cobalt containing material composition of metal surface (M).

As a fifth example, for a material composition (as described hereinabove) including a heavy metal in metal surface (M) of medical implant component 12, among the entire hereinabove list of chelator (C) compounds, each of the following: 2,3-dimercapto-1-propansulfonic acid (DMPS); mecaptosuccinic acid (DMSA); S-cystaminyl-EDTA; amino tris methylenephosphoric acid (ATMA); and 1-hydroxyethylidene-1-bisphosphonate (HEBP), is known to have structure and function as a multi-dentate or multifunctional ligand for complexing or chelating to (coordinating with) the heavy metal metal ions (or atoms) of the heavy metal containing material composition of metal surface (M).

Within the scope of the present invention, in a non-limiting manner, it is to be fully understood that, in addition to the above list of chelator (C) compounds, many other types of chelator (C) compounds bound in the surface coating region in the form of a surface coating, are suitable for implementing the present invention.

Additional Properties, Characteristics, and Aspects, of Chemical Entity (X)

Chemical entity (X), also as part of the surface coating region in the form of a surface coating, is composed of any single type, or combination of two or more single types, of species (uncharged or charged atoms or molecules), for example, d1, L1, d2, d3, L2, and d4, as illustrated in FIGS. 1 and 2. Each chemical entity specie, for example, d1, L1, d3, and L2, of chemical entity (X), has the potential for bonding to, or at least interacting in a bonding-like (affinity) manner with, a metal chelated (complexed) chelator molecule, for example, c2, c3, c4, and c5, of chelator (C). The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof. In an exemplary preferred embodiment of the present invention, the bonding or the at least bonding-like (affinity) interaction, is either stable (that is, not ordinarily cleavable or breakable), or is cleavable or breakable via an appropriate bond or bond-like cleaving or breaking mechanism (for example, enzymatic reaction) and an appropriately corresponding bond or bond-like cleaving or breaking agent (for example, an enzyme), resulting in separation, release or elution, and subsequent migration, of the corresponding chemical entity specie d1, L1, d3, and L2, respectively, of chemical entity (X), away from the corresponding metal chelated (complexed) chelator molecule c2, c3, c4, and c5, respectively, of chelator (C), as previously illustratively described and exemplified with reference to FIGS. 1, 2, and 3.

Preferably, each chemical entity specie, for example, L1 and L2, of chemical entity (X), which is chelated (complexed) to a chelator molecule, for example, c3 and c5, respectively, of chelator (C), has the potential for bonding to, or at least interacting in a bonding-like (affinity) manner with, an additional chemical entity specie, for example, d2 and d4, respectively, of chemical entity (X), as illustrated in FIGS. 1 and 2. The bonding can be at least one covalent bond, at least one ionic bond, at least one hydrogen bond, at least one van der Waals bond, at least one coordinate covalent bond, or a combination thereof. The bonding-like (affinity) interaction can be of a dipole-dipole type, a hydrophilic type, a hydrophobic type, or a combination thereof. In an exemplary preferred embodiment of the present invention, the bonding or the at least bonding-like (affinity) interaction, is either stable (that is, not ordinarily cleavable or breakable), or is cleavable or breakable via an appropriate bond or bond-like cleaving or breaking mechanism (for example, enzymatic reaction, or chemical reaction) and an appropriately corresponding bond or bond-like cleaving or breaking agent (for example, an enzyme, or other chemical type bond or bond-like cleaving or breaking agent), resulting in separation, release or elution, and subsequent migration, of the corresponding additional chemical entity specie d2 and d4, respectively, away from the corresponding (chelator bonded or interacting) chemical entity specie L1 and L2, respectively, of chemical entity (X), as previously illustratively described and exemplified with reference to FIGS. 1, 2, and 3.

Consistent with the hereinabove illustrative description of the present invention, in general, chemical entities which are suitable for applying the present invention are essentially any of a wide variety of different categories and types of chemical compounds. Exemplary chemical entities are a drug, a biological entity, a linker or spacer capable of binding a drug or a biological entity, and a linker or spacer to which a drug or a biological entity is bound.

Chemical Entity (X) as a Drug

An exemplary specific type of chemical entity specie of chemical entity (X), which is suitable for implementing the present invention, is a drug (uncharged or charged molecule) (hereinabove, exemplified and referred to in the text and in FIGS. 1, 2, and 3, as chemical entity species d1, d2, d3, and d4). A preferred exemplary type of drug is a drug used for preventing or/and treating a medical condition, such as a cardiovascular type of medical condition, of a subject. Exemplary cardiovascular types of a medical condition of a subject are restenosis, in general, and in-stent restenosis, in particular, and thrombosis. Accordingly, an exemplary type of drug used for preventing or/and treating a cardiovascular type of medical condition of a subject is a cardiovascular drug. An exemplary type of cardiovascular drug is a drug that prevents or/and inhibits onset or/and progression of restenosis, in general, and in-stent restenosis, in particular. Another exemplary type of cardiovascular drug is a drug that prevents or/and inhibits onset or/and progression of thrombosis.

Examples of cardiovascular drugs which are suitable for implementing the present invention, are: (I) alpha-adrenergic blocking drugs (alpha blocking drugs), for example, doxazosin (Cardura), and iabetolol (Normodyne, Trandate); (2) angiotensin converting enzyme (ace) inhibitor drugs, for example, captopril (Capoten), enalapril (Vasotec), and lisinopril (Prinivil, Zestril); (3) antiarrhythmic drugs, for example, amiodarone (Cordarone), digoxin (Lanoxin), disopyramide phosphate (Norpace), flecainide (Tambocor), lidocaine (Xylocalne), mexiletine (Mexitil), procainamide (Procan SR, Pronestyl, Pronestyl SR), quinidine gluconate (Duraquin, Quinaglute Dura-Tabs, Quinalan Sustained-Release) quinidine sulfate (Quinidex Extentabs), and tocainide (Tonocard); (4) anticoagulant and antiplatelet (anticlotting) drugs, for example, acetylsalicylic acid or aspirin (Alka-Seltzer, Anacin, Ascriptin, Bayer, Bufferin, Easprin, Ecotrin, St. Josephs, Zorprin), dipyridamole (Persantine), warfarin (Coumadin, Panwarfin), thienopyridines (Ticlopidine, Clopidogrel), and glycoprotein IIb/IIIa receptor inhibitors or antagonists (Abciximab, Eptifibatide, Tirofiban); (5) antithrombotic or thrombin inhibitor drugs (Heparin, Hirudin, Bivalirudin, Lepirudin, Argatroban); (6) beta-adrenergic blocking drugs (beta blocking drugs), for example, acebutolol (Sectral), atenolol (Tenormin), metoprolol (Lopressor), nadolol (Corgard), pindolol (Visken), and propranolol (Inderal); (7) calcium channel blocking drugs, for example, diltiazem (Cardizem), nicardipine (Cardene), nifedipine (Procardia, Procardia XL), nimodipine (Nimotop), and verapamil (Calan, Isoptin, Verelan); (8) centrally acting drugs, for example, clonidine (Catapres, Catapres-TTS), guanabenz (Wytensin), guanfacine (Tenex), and methyldopa (Aldomet); (9) cholesterol lowering agent drugs, for example, cholestyramine (Questran, Questran Light) colestipol (Colestid), gemfibrozil (Lopid), lovastatin (Mevacor), nicotinic acid, niacin (Nia-Bid, Niacels, Niacor, Niaplus, Nicolar, Nicobid, Slo-Niacin), and probucol (Lorelco); (10) digitalis drugs, for example, digoxin (Lanoxicaps, Lanoxin), and digitoxin (Crystodigin, Purodigin); (11) diuretic drugs, for example, chlorthalidone (Hygroton), hydrochlorothiazide (Esidrix, Hydrodiuril, Oretic), metolazone (Diulo, Mykrox, Zaroxolyn), bumetamide (Bumex), furosemide (Lasix), amiloride (Midamor), spironolactone (Aldactone), and triamterene (Dyrenium); (12) nitrate drugs, for example, nitroglycerin (Deponit NTG, Minitran, Nitro-Bid, Nitrogard, Nitroglyn, Nitrol, Nitrolingual, Nitrong, Nitrostat, Transderm-Nitro, Tridil), and isosorbide dinitrate (Dilatrate-SR, Iso-Bid, Isordil, Sorbitrate, Sorbitrate SA); (13) peripheral adrenergic antagonist drugs, for example, reserpine (Serpasil); (14) vasodilator drugs, for example, hydralazine (Apresoline), and minoxidil (Loniten); (15) combination drugs, for example, amiloride-hydrochlorothiazide (Moduretic), atenolo-chlorthalidone (Tenoretic), captopril-hydrochlorothiazide (Capozide), clonidine-chlorthalidone (Combipres), chlorthalidone-reserpine (Demi-Regroton, Regroton), enalapril-hydrochlorothiazide (Vaseretic), hydralazine-hydrochlorothiazide (Apresazide), hydrochlorothiazide-reserpine (Hydropres), labetolol-hydrochlorothiazide (Normozide, Trandate HCT), lisinopril-hydrochlorothiazide (Zestoretic), methyldopa-hydrochlorothiazide (Aldoril), propranolol-hydrochlorothiazide (Inderide, Inderide LA), reserpine-hydralazinel hydrochlorothiazide (Ser-Ap-Es), spironolactone-hydrochlorothiazide (Aldactazide), and triamterene-hydrochlorothiazide (Dyazide, Maxzide), and (16) combination drugs thereof.

Examples of drugs that prevent or/and inhibit onset or/and progression of restenosis, in general, and in-stent restenosis, in particular, and which are suitable for implementing the present invention, are: (1) anti-neoplastic (anti-inflammatory) drugs, for example, dexamethasone, m-prednisolone, interferon gamma-1b, leflunomide, sirolimus (and analogs), tacrolimus, mycophenolic acid, mizoribine, cyclosporine, Tranilast, and Biorest; (2) immunosupressive (anti-proliferative) drugs, for example, QP-2, taxol, actinomycin, methothrexate, angiopeptin, vincristine, mitomycine, statins, ‘c myc c myc’ antisense antisense, sirolimus (and analogs), restenASE 2-chlorodeoxyadenosine, and PCNA ribozyme; (3) migration inhibitor (ECM modulator) drugs, for example, batimastat, prolyl hydroxylase inhibitors, halofuginone, C-proteinase inhibitors, and probucol; and (4) enhanced healing (re-endothelialization) drugs, for example, BCP671, VEGFs (vascular endothelial growth factors), FGFs (fibroblast growth factors), estradiols, NO donors, EPC (endothelial progenitor cells), antibodies, Biorest, and advanced coatings.

Within the scope of the present invention, in a non-limiting manner, it is to be fully understood that, in addition to the above list of drug type chemical entity species of chemical entity (X), many other drug type chemical entity species of chemical entity (X), are suitable for implementing the present invention.

Chemical Entity (X) as a Biological Moiety

Another exemplary specific type of chemical entity specie of chemical entity (X), which is suitable for implementing the present invention, is a biological moiety (uncharged or charged molecule) (hereinabove, alternatively exemplified and referred to in the text and in FIGS. 1, 2, and 3, as chemical entity species d1, d2, d3, and d4). Herein, a biological moiety refers to a part or portion (of indefinite size or/and structure) of a biological entity, wherein a biological entity refers to an entity, a material, a substance, or a structure, originating or derived from a biological (human, animal, or plant) organism.

Exemplary biological moiety type chemical entity species of chemical entity (X) which are suitable for implementing the present invention are selected from the group consisting of proteins, lipids (fats), sugars, nucleic acids, antibodies, cells, cellular structures, cellular components, and combinations thereof.

Exemplary proteins are selected from the group consisting of enzymes, growth factors, hormones, cytokines, and combinations thereof. Exemplary enzymes are selected from the group consisting of serine protease, matrix metalloproteinases, aspartic proteinases, and combinations thereof. Exemplary growth factors are selected from the group consisting of vascular endothelial growth factors (VEGFs), platelet derived growth factors (PDGFs), bone morphogenetic proteins (BMPs), and combinations thereof. Exemplary hormones are selected from the group consisting of Interleukin-1, Interleukin-2, growth hormones, and combinations thereof. Exemplary cytokines are selected from the group consisting of growth related oncogenes (GROs), interferon-inducible protein-10 (IP-10), neutrophil activating protein-2 (NAP-2), and combinations thereof.

Exemplary lipids (fats) are selected from the group consisting of phospholipids, glycolipids, steroids, and combinations thereof.

Exemplary sugars are selected from the group consisting of heparin, chondritin, glycogen, and combinations thereof.

Exemplary nucleic acids are selected from the group consisting of deoxoribonucleic acid (DNA), ribonucleic acid (RNA), peptide nucleic acid (PNA), and combinations thereof.

Exemplary antibodies are selected from the group consisting of polyclonal antibodies, monoclonal antibodies, Fab fragments, and combinations thereof.

An exemplary biological entity (material, substance, or structure), from which any of the above stated biological moieties originates or is derived, is a cell, a cellular structure, or a cellular component. In a non-limiting manner, exemplary cells are embryonic stem cells, fetal stem cells, and adult stem cells. Such stem cells originate or are derived from essentially any biological source or organ. In a non-limiting manner, exemplary stem cells are hematopoietic stem cells, liver stem cells, mesenchymal stem cells.

Adult stem cells usually divide to generate progenitor or precursor cells, which then differentiate or develop into ‘mature’ cell types that have characteristic shapes and specialized functions, e.g., muscle cell contraction or nerve cell signaling. Exemplary sources of adult stem cells are bone marrow, blood, the cornea and the retina of the eye, the brain, skeletal muscle, dental pulp, liver, skin, the lining of the gastrointestinal tract, and pancreas.

Any of the above indicated types of cells can be selected so as to produce and secrete one or more biological moieties, e.g., any one or more of the hereinabove stated types of biological moieties, i.e., selected from the group consisting of proteins, lipids (fats), sugars, nucleic acids, antibodies, and combinations thereof. Moreover, any of the above indicated types of cells can be either naive and naturally produce and secrete one or more biological moieties, or the cell can be transformed or converted in such a manner as to produce and secrete one or more biological moieties.

Within the scope of the present invention, in a non-limiting manner, it is to be fully understood that, in addition to the above indicated biological moiety type chemical entity species of chemical entity (X), many other biological moiety type chemical entity species of chemical entity (X), are suitable for implementing the present invention.

Chemical Entity (X) as a Linker or Spacer

Another exemplary specific type of chemical entity specie of chemical entity (X), which is suitable for implementing the present invention, is a linker (uncharged or charged atom or molecule), also known and referred to as a spacer (herein, exemplified and referred to in the text and in FIGS. 1, 2, and 3, as chemical entity species or linkers L1 and L2). The linker or spacer is either biodegradable or non-biodegradable. A preferred type of linker or spacer is selected from the group consisting of peptides, lipids, and sugars. An exemplary peptide, lipid, or sugar, type of linker or spacer is a peptide, lipid, or sugar, respectively, that is a substrate to, and is cleavable or breakable by, at least one type of an enzyme (protease, lipase, or sugar degrading enzyme, respectively) whose activity is induced or expressed during onset of restenosis, in general, and in-stent restenosis, in particular, or/and thrombosis, which typically occur to varying extents following treatment of intravascular ailments and diseases via interventional procedures of angioplasty and stent implantation.

As previously stated in the Background section, regarding the pathology and biochemistry of restenosis, in general, and in-stent restenosis, in particular, the extracellular matrix (ECM) consists mainly of fibrous proteins and structured sugars. ECM fibrous proteins are of two functional types: structural, such as collagen and elastin, and adhesive, such as fibronectin and laminine. ECM structured sugars are mainly polysaccharide glycosaminoglycans, such as hyaluronic acid, chlondroitin sulfate, dermatan sulfate, heparan sulfate, heparin, and keratan sulfate [Hay, E. D., 1981; McDonald, J. A., 1988; Piez, K. A., et al., 1984]. ECM remodeling involves a wide variety of different types of enzymes that control the process. Exemplary ECM remodeling types of enzymes are proteases, such as matrix metalloproteinases (MMPs), serine-type peptidases, threonine-type peptidases, aspartic-type peptidases, and cystein-type peptidases. Other enzymes, such as lipid or sugar degrading enzymes, also can play a role in extracellular matrix remodeling, among them enzymes that degrade structured sugars of the matrix, such as heparinase and hyaloronidase.

Major drivers that induce vascular remodeling and matrix metalloproteinase (MMP) expression and activation are: injury, inflammation, and oxidative stress. All these factors play an important role in restenosis, in general, and in-stent restenosis, in particular. Many different types of matrix metalloproteinases (MMPs) are involved in vascular remodeling and atherogenesis. MMPs that were shown to be involved in vascular remodeling are: MMP-1, MMP-2, MMP-3, MMP-7, MMP-9, MMP-12, MMP-13, and MMP-14 [Zorina, S., et al., 2002]. All of these MMPs are produced by human macrophage cells. MMP-1, 2, 3, 9, and 14, are produced by SMCs both in-vitro and in animal studies. There are animal studies that show differential expression of MMPs after stent implantation and balloon injury.

There is extensive evidence suggesting that SMCs produce plasminogen activators and MMPs in response to vessel wall injury [Clowes, A. W., 1990; Jackson., C. L., 1993; Zempo, N., et al., 1994; Reidy, M. A., et al., 1996; Shofuda, K., et al., 1998]. For example, arterial injury causes expression and activation of MMP-2 and MMP-9, and this is associated with increased migration and proliferation of SMCs [Zempo, N., et al., 1994; Bendeck, M. P., 1994]. Several other MMPs are also expressed in human atherosclerotic lesions, including stromelysin (MMP-3), interstitial collagenase (MMP-1) and type IV collagenases (MMP-2 and MMP-9) [Henney, A., et al., 1991; Galis, Z. S., et al., 1994; Brown, D. L., et al., 1995].

Intimal hyperplasia is the principal mechanism of restenosis, in general, and in-stent restenosis, in particular. Studies of MMP expression following stent implantation show over-expression of MMP-9 and activation of MMP-2 in animal models [Feldman, L. J., et al., 2001]. Neointima formation in organ cultured human Saphenous vein grafts is inhibited by simvastatin (investigational new drug (IND)), and is associated with MMP-9 reduced activity and inhibition of SMC proliferation and migration [Porter, K. E., et al., 2002]. FUT-175, a serine protease inhibitor, also inhibits neointimal formation after balloon injury in rats [Sawada, M., et al., 1999].

Many MMP substrates and inhibitors have been identified [Whittaker, M., et al., 1999]. Most of MMP substrates are native proteins of the ECM in which the specific peptide sequence that is being cleaved was identified [Netzel-Arnett, S., et al., JBC, 1991; Netzel-Amett, S., Anal. Biochem., 1991; Niedzwiecki, L., et al., 1992].

Accordingly, for implementing the present invention, preferably, the peptide type of linker or spacer is a peptide that is a substrate to, and is cleavable by, a matrix metalloproteinase (MMP) protease type of enzyme, whose activity is induced or expressed during onset of restenosis, in general, and in-stent restenosis, in particular, or/and thrombosis. An exemplary type of peptide linker or spacer, which is suitable for implementing the present invention, is a matrix metalloproteinase (MMP) substrate selected from the group consisting of (1) a substrate of MMP-9, for example, Pro-Arg-Ser/Thr-Hy (Ala, Leu, Ile, Met, Val, Phe)-Ser/Thr [Kridel, S. J., 2001]; (2) a substrate of MMP-2, for example, Pro-Leu-Ala-Nva-Dpa-Ala-Arg [Murphy, G., et. al., 1994]; (3) a substrate of MMP-3, for example, Pro-Tyr-Ala-Tyr-Trp-Met-Arg [Netzel-Arnett, S., et. al., 1991, 195]; (4) a substrate of MMP-14, for example, Pro-Leu-Ala-Cys-Trp-Ala-Arg [Mucha, A., et. al., 1998]; and (5) a substrate of MMP-1, for example, Pro-Leu-Gly-Met-Trp-Ser-Arg [Netzel-Amett, S., et. al., 1993].

Additional exemplary types of peptide linkers or spacers, which are suitable for implementing the present invention, are peptides that are substrates to, and cleavable by, a type of peptidase selected from the group consisting of serine-type peptidases, threonine-type peptidases, aspartic-type peptidases, and cystein-type peptidases.

Exemplary types of lipid linkers or spacers, which are suitable for implementing the present invention, are lipids selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-minoheptanoic acid, 6-aminohexanoic acid, and 4-aminobutyric acid.

Exemplary types of sugar linkers or spacers, which are suitable for implementing the present invention, are sugars selected from the group consisting of polysaccharide glycosaminoglycans (for example, hyaluronic acid), chlondroitin sulfate, dermatan sulfate, heparan sulfate, heparin, and keratan sulfate.

Another preferred exemplary type of a linker or spacer is a biocompatible synthetic polymer. Preferably, the biocompatible synthetic polymer type of linker or spacer is a biocompatible synthetic polymer that is a substrate to, and is cleavable or breakable by, at least one type of a chemical (for example, an oxidative agent, such as nitric oxide, that can cleave or break disulfide (S—S) bonds in a synthetic polymer) whose activity is induced or expressed during onset of restenosis, in general, and in-stent restenosis, in particular, or/and thrombosis. Exemplary types of biocompatible synthetic polymer linkers or spacers, which are suitable for implementing the present invention, are biocompatible synthetic polymers selected from the group consisting of synthetic polyethylene glycols (PEGs). Preferred exemplary synthetic polyethylene glycols are polyethylene glycol 400 (PEG-400), polyethylene glycol 200 (PEG-200), polyethylene glycol-distearoylphosphatidylethanolamine (PEG-DSPE), polyethylene glycol-caprolactone/trimethylenecarbonate (PEG-CAP/TMC) copolymers, polyethylene glycol-(poly-lactic acid) (PEG-PLA), S-nitrosylated polyethylene glycol (SNO-polyethylene glycol), methoxy-polyethylene glycol (MeO-PEG), and dimyristoylphosphatidylethanolamine-N-[methoxy(polyethylene glycol)] (DMPE-PEG).

Another preferred exemplary type of a linker or spacer is a biocompatible synthetic bi-functional cross-linker. As used herein, a bi-functional cross-linker is a type of cross-linker whose molecules each have two reactive ends (with the same or different functionalities) that specifically react with functional groups, such as primary amines, sulphydryls, carboxyls, etc., present in other molecular species, attaching to those functional groups via a covalently bonded bridge type configuration.

Preferably, the biocompatible synthetic bi-functional cross-linker type of linker or spacer is a biocompatible synthetic bi-functional cross-linker that is a substrate to, and is cleavable or breakable by, at least one type of a chemical (for example, an oxidative agent, such as nitric oxide, that can cleave or break disulfide (S—S) bonds in a synthetic bi-functional cross-linker) whose activity is induced or expressed during onset of restenosis, in general, and in-stent restenosis, in particular, or/and thrombosis. Exemplary types of biocompatible synthetic bi-functional cross-linker linkers or spacers, which are suitable for implementing the present invention, are biocompatible synthetic bi-functional cross-linkers selected from the group consisting of synthetic m-maleimido-N-hydroxysuccinimide (BMS), bis[beta-(4-azidosalicylamido)ethyl]disulfide (BASED), bis-maleimidohexane (BMH), and sulfosuccinimidyl-[perfluoroazidobenzamido]-ethyl-1,3-dinitropropionate (SFAD).

Within the scope of the present invention, in a non-limiting manner, it is to be fully understood that, in addition to the above list of linker or spacer type chemical entity species of chemical entity (X), many other linker or spacer type chemical entity species of chemical entity (X), are suitable for implementing the present invention.

Within the scope of the present invention, in a non-limiting manner, it is to be fully understood that in any given embodiment or configuration of ‘metal chelated surface’ medical device 10, 10′, or 10″ (FIGS. 1, 2, and 3, respectively), of the present invention, the hereinabove listed exemplary chelator compounds of chelator (C), and the above listed chemical entity species of chemical entity (X), are potentially interchangeable or substitutable by each other, whereby a given chelator compound of chelator (C) may exhibit structure, function, and behavior, of a chemical entity specie of chemical entity (X), and vice versa, whereby a given chemical entity specie of chemical entity (X) may exhibit structure, function, and behavior, of a chelator compound of chelator (C). Moreover, for example, any of the hereinabove listed exemplary drug or biological moiety type of chemical entity specie of chemical entity (X) may exhibit structure, function, and behavior, of a linker or spacer type of chemical entity specie of chemical entity (X), and vice versa, any of the above listed linker or spacer type of chemical entity specie of chemical entity (X), may exhibit structure, function, and behavior, of a drug or a biological moiety type of chemical entity specie of chemical entity (X).

According to another main aspect of the present invention, there is provided a method of manufacturing an implantable medical device characterized by including the step of binding to a metal surface (M) of a medical implant component a chemical entity (X) via a chelator (C) in an (M)-(C)-(X) configuration. Accordingly, the method of manufacturing an implantable medical device, for example, ‘metal chelated surface’ medical device, 10 or 10′ (FIGS. 1 and 2, respectively), of the present invention, is characterized by including the step of binding to metal surface (M) of medical implant component 12, chemical entity (X), via chelator (C), in an (M)-(C)-(X) configuration.

In this step of the method for manufacturing the ‘metal chelated surface’ medical device, 10 or 10′ (FIGS. 1 and 2, respectively), of the present invention, there is binding to metal surface (M) of medical implant component 12, chemical entity species (uncharged or charged atoms or/and molecules), for example, d1, L1, d2, d3, L2, and d4, of chemical entity (X), singly or/and in combination, via metal chelated (complexed) chelator molecules, for example, c2, c3, c4, and c5, respectively, of chelator (C), in an (M)-(C)-(X) configuration, for forming, for example, metal surface (M)-chelator (C)-chemical entity (X) chelate type of coordination compound configurations m2-c2-d1, m4-c3-L1, m7-c4-d3, and m8-c5-L2, or/and, for forming, for example, metal surface (M)-chelator (C)-chemical entity (X) chelate type of coordination compound configurations m4-c3-L1-d2, and m8-c5-L2-d4, as illustrated in FIGS. 1 and 2.

As previously stated hereinabove, within the scope of the present invention, in a non-limiting manner, it is to be fully understood that for a given set of parameters of previous and current physicochemical treatments or/and conditions, metal surface (M) of medical implant component 12 of ‘metal chelated surface’ medical device 10 or 10′ (FIGS. 1 and 2, respectively), can include any number of different possible types of sub-populations and configurations of exposed surface metal ions and atoms of metal surface (M) which are charged (cationic or anionic), uncharged (neutral), or polarized, and which are either chelated (complexed) or not chelated to a chelator molecule of chelator (C).

Additionally, metal surface (M) of medical implant component 12 can include any number of different possible types of sub-populations and configurations of chelated (complexed) chelator molecules of chelator (C) which are bonded or non-bonded to chemical entity species of chemical entity (X), or/and include any number of different types of sub-populations and configurations of chemical entity species of chemical entity (X) which are bonded to chelated (complexed) chelator molecules of chelator (C), or/and which are bonded to other chemical entity species of chemical entity (X).

Additionally, there is a wide variety of different possible types of chemical entity species (uncharged or charged atoms or/and molecules) of chemical entity (X), including for example, drug, biological moiety, or other types of chemical entity species, for example, d1, d2, d3, and d4, and including, for example, linker or spacer (uncharged or charged atom or molecule) types of chemical entity species, for example, L1 and L2, wherein each of such type of chemical entity species is bonded to, or at least interacts in a bonding-like (affinity) manner with, the chelator molecules of chelator (C), or/and with each other, and wherein the various types of bonding can be at least one covalent bond, at least one ionic bond, at least one hydrogen bond, at least one van der Waals bond, at least one coordinate covalent bond, or a combination thereof, and wherein the various type of bonding-like (affinity) interaction can be of a dipole-dipole type, a hydrophilic type, a hydrophobic type, or a combination thereof.

Accordingly, there is a correspondingly wide variety of different possible sub-steps, and, sequences and orders thereof, for performing this step of binding to metal surface (M) of medical implant component 12, chemical entity species of chemical entity (X), singly or/and in combination, via metal chelated (complexed) chelator molecules of chelator (C), in an (M)-(C)-(X) configuration, for forming the metal surface (M)-chelator (C)-chemical entity (X) chelate type of coordination compound configurations.

For the objective of illustrating implementation of the present invention, in a non-limiting manner, there is illustratively described herein, an exemplary preferred embodiment of the method for manufacturing the ‘metal chelated surface’ medical device, 10 or 10′ (FIGS. 1 and 2, respectively), of the present invention, wherein the step of binding to metal surface (M) of medical implant component 12, chemical entity species of chemical entity (X), via metal chelated (complexed) chelator molecules of chelator (C), in an (M)-(C)-(X) configuration, includes the following sequence or order of sub-steps: removing metal surface blocking from metal surface (M) of medical implant component 12; activating (via ionizing and charging) metal surface (M), for forming an activated (ionized and charged) metal surface (M) which is capable of being chelated (complexed) to chelator (C), and therefore, for binding chelator (C); binding (via chelation) of chelator (C) to the activated (ionized and charged) metal surface (M) of medical implant component 12, for forming medical implant component 12 having metal surface (M) to which is chelated chelator (C) in an (M)-(C) chelate type of coordination compound configuration; reactively combining a first chemical entity specie of chemical entity (X), with a second chemical entity specie of chemical entity (X), for forming a third (combination) chemical entity specie of chemical entity (X); and, binding the third (combination) chemical entity specie of chemical entity (X) to the metal chelated (complexed) chelator (C) which is bound to metal surface (M).

The step, and sub-steps thereof, of binding to metal surface (M), chemical entity species of chemical entity (X), singly or/and in combination, via chelator molecules of chelator (C) which are chelated (complexed) to metal surface (M), are performed by using chemical or/and electrochemical types of procedures. General details and exemplary conditions of performing chemical and electrochemical types of procedures for implementing this step, and sub-steps thereof, are provided immediately below. Specific examples of implementing this step, and sub-steps thereof, are provided in Examples 1-11, in the Examples section hereinbelow.

Removing Metal Surface Blocking from the Metal Surface (M)

Typically, especially the case during the manufacture of stent types of medical implant devices, the metal surface, in general, and the uppermost or exposed surface metal atoms of the metal surface, in particular, of the stent, are initially blocked, shielded, or covered, for example, by a layer or coating of hydrocarbons or/and by a layer or coating of deposited phosphoric acid ions or/and sulfuric acid ions, as a result of the metal surface previously having been subjected to a surface electro-polishing procedure or/and to a surface passivation procedure.

Accordingly, for an embodiment of medical implant component 12, wherein metal surface (M), in general, and uppermost or exposed surface metal atoms m1-m11 of metal surface (M), in particular, are initially blocked, shielded, or covered, for example, by a layer or coating of hydrocarbons or/and by a layer or coating of deposited phosphoric acid ions or/and sulfuric acid ions, as a result of metal surface (M) previously having been subjected to a surface electro-polishing procedure or/and to a surface passivation procedure, then, preferably, as an initial sub-step of the manufacturing method, there is removing metal surface blocking from metal surface (M), prior to performing the step of binding chemical entity (X), via chelator (C), to metal surface (M) of medical implant component 12 in an (M)-(C)-(X) configuration.

The sub-step of removing metal surface blocking from metal surface (M) of medical implant component 12, is preferably performed by exposing metal surface (M) to a base (caustic reagent) in liquid phase at mild conditions, followed by fully washing the base treated metal surface (M) several times with water. The base is either an inorganic base or an organic base. Exemplary inorganic bases are ammonium hydroxide (NH₄OH), sodium hydroxide (NaOH), and potassium hydroxide (KOH). For such inorganic bases, exemplary mild conditions correspond to exposing metal surface (M) to a dilute aqueous solution of a concentrated base, with the final concentrated base concentration in a range of between about 5% and about 30% (vol/vol), at room temperature (20-25° C.), for about 30 minutes, followed by fully washing the base treated metal surface (M) several times with water. Exemplary liquid phase organic bases are piperidine, pyridine, triethylamine, propylamine, diisopropilamine, and dimethylaminoperidine. For such organic bases, exemplary mild conditions correspond to exposing metal surface (M) to the liquid phase base at room temperature (20-25° C.), for about 30 minutes, followed by fully washing the base treated metal surface (M) several times with water. Metal surface (M), absent of the metal surface blocking, is then ready for being subjected to the next sub-step of activating (via ionizing and charging) metal surface (M), for forming an activated (ionized and charged) metal surface (M).

A specific example of performing this sub-step is provided in Example 1, in the Examples section hereinbelow.

Alternatively, for an embodiment of medical implant component 12, wherein metal surface (M), in general, and uppermost or exposed surface metal atoms m1-m11 of metal surface (M), in particular, are ‘not’ initially blocked, shielded, or covered, for example, by a layer or coating of hydrocarbons or/and by a layer or coating of deposited phosphoric acid ions or/and sulfuric acid ions, as a result of metal surface (M) previously not having been subjected to a surface electro-polishing procedure or/and to a surface passivation procedure, then, preferably, instead of performing the just described sub-step for removing metal surface blocking from metal surface (M), there is performing the next sub-step of activating (via ionizing and charging) metal surface (M), for forming an activated (ionized and charged) metal surface (M).

Activating (Via Ionizing and Charging) the Metal Surface (M)

For metal surface (M) absent of metal surface blocking, then, in this sub-step of the method for manufacturing the ‘metal chelated surface’ medical device, for example, 10 or 10′ (FIGS. 1 and 2, respectively), of the present invention, there is activating (via ionizing and charging) metal surface (M), for forming an activated (ionized and charged) metal surface (M) which is capable of being chelated (complexed) to chelator (C), and therefore, for binding chelator (C). Accordingly, in this sub-step, there is activating metal surface (M), in general, and uppermost or exposed surface metal atoms m1-m11 of metal surface (M), in particular, of medical implant component 12.

Activating (via ionizing and charging) metal surface (M) is performed by using a suitable type of metal surface activation procedure, for example, a chemical type of metal surface activation procedure, or an electrochemical type of metal surface activation procedure. Either metal surface activation procedure is performed for the objective of oxidizing or reducing metal surface (M), in general, and for oxidizing or reducing at least a sub-population of exposed surface metal atoms m1-m11 of metal surface (M), in particular, for forming an activated (ionized and charged, oxidized or reduced) metal surface (M), in general, which is capable of being chelated (complexed) to chelator (C), and for forming a sub-population of exposed surface metal ions m1, m2, m4, m5, m7, m8, m10, and m11, of metal surface (M), in particular, each of which is ionized and charged (cationic or anionic) and capable of being chelated (complexed) to, and therefore, binding, one or more chelator molecules c1-c6 of chelator (C).

Exemplary chemical types of a metal surface activation procedure, which are suitable for implementing the present invention, are based on chemical oxidation involving use of at least one chemical oxidant (oxidizing reagent), or chemical reduction involving use of at least one chemical reducer (reducing reagent). The actual chemical type (oxidation or reduction) of metal surface activation procedure is selected according to, and for being electronically compatible with, the charge (negative or positive) and ionic state (anionic or cationic, respectively) of chelator molecules c1-c6, of chelator (C) which are to be used for chelating (complexing) and binding to the sub-population of exposed surface metal ions m1, m2, m4, m5, m7, m8, m10, and m11, of metal surface (M). Typically, in a non-limiting manner, a chemical oxidation type of metal surface activation procedure is used, involving the use of at least one chemical oxidant (oxidizing reagent), for activating (oxidizing) metal surface (M), in general, and for activating (oxidizing) at least a sub-population of exposed surface metal atoms m1-m11 of metal surface (M), for forming a positively charged metal surface (M) having a sub-population of positively charged (cationic) exposed surface metal ions (cations), for example, m1, m2, m4, m5, m7, m8, m10, and m11, which are to be chelated (complexed) to a negatively charged chelator (C) having negatively charged (anionic) chelator molecules (anions), for example, c1-c6.

Examples of chemical oxidants (oxidizing reagents) usable in a chemical oxidation type of a metal surface activation procedure, and which are suitable for implementing the present invention, are: chromates, for example, potassium dichromate+sulfuric acid (K₂Cr₂O₇+H₂SO₄); nitrates, for example, sodium nitrate (NaNO₃); nitrites, for example, sodium nitrite (NaNO₂); persulfates, for example, ammonium persulfate ((NH₄)₂S₂O₈), potassium persulfate (K₂S₂O₈); permanganates, for example, potassium permanganate (KMnO₄); periodates, for example, sodium periodate (NalO₄); oxygen (O₂); hydrogen peroxide (H₂O₂), and combinations thereof. Exemplary conditions of applying the chemical oxidation type of a metal surface activation procedure, include exposing metal surface (M) of medical implant component 12 to one or more liquid phase chemical oxidants (oxidizing reagents) at a temperature in a range of between about 20° C. and about 100° C., and preferably, in a range of between about 70° C. and about 100° C., for about 20 minutes, followed by fully washing the activated (ionized and charged, oxidized) metal surface (M) several times with water. A specific example of performing the chemical oxidation type of a metal surface activation procedure of this sub-step is provided in Example 2, in the Examples section hereinbelow.

Exemplary electrochemical types of a metal surface activation procedure, which are suitable for implementing the present invention, are based on electrochemical oxidation or reduction of metal surface (M) taking place in an electrochemical cell which houses an electrolytic fluid or bath including at least one chemical oxidant (oxidizing reagent), or, at least one chemical reducer (reducing reagent). Metal surface (M) of medical implant component 12 is electrochemically exposed to the electrolytic fluid or bath and is conductively attached or connected to the first electrode terminal (anode or cathode, for oxidation or reduction, respectively), with a non-corrosive metallic element being conductively attached or connected to the corresponding second electrode terminal (cathode or anode, for oxidation or reduction, respectively). Similar to the chemical type of metal surface activation procedure, the actual electrochemical type (oxidation or reduction) of metal surface activation procedure is selected according to, and for being electronically compatible with, the charge (negative or positive) and ionic state (anionic or cationic, respectively) of chelator molecules c1-c6, of chelator (C) which are to be used for chelating (complexing) and binding to the sub-population of exposed surface metal ions m1, m2, m4, m5, m7, m8, m10, and m11, of metal surface (M).

Exemplary conditions of applying the electrochemical oxidation type of a metal surface activation procedure, include immersing metal surface (M) of medical implant component 12 into an electrochemical cell which houses an aqueous electrolytic fluid or bath including at least one chemical oxidant (oxidizing reagent) each at a molar concentration of, for example, about 0.5-1 M. Metal surface (M) of medical implant component 12 is electrochemically exposed to the electrolytic fluid or bath and is conductively attached or connected to the anode electrode terminal (for oxidation), with a non-corrosive metallic element being conductively attached or connected to the corresponding cathode electrode terminal, with the ratio of cathode surface area and anode surface area preferably being at least about two to one. Current density in a range of between about 0.5 amps per square inch and about 200 amps per square inch is maintained between metal surface (M) of medical implant component 12 and the cathode, during the electrolysis procedure, which is performed for between about 5 and 60 minutes at a temperature in a range of between about −20° C. and about 80° C. Following the electrolysis procedure, metal surface (M) is fully washed in an appropriate solvent, for example, an alcohol/water, 1/1 (vol/vol), solution, and dried.

Examples of chemical oxidants (oxidizing reagents) usable in an electrochemical oxidation type of a metal surface activation procedure, and which are suitable for implementing the present invention, are: hydrochloric acid (HCl), hydrobromic acid (HBr), hydrofluoric acid (HF), sulfuric acid (H₂SO₄), phosphoric acid (H₃PO₄), perchloric acid (HClO₄), trifluoroacetic acid (CF₃COOH), oxalic acid (H₂C₂O₄), citric acid (C₆H₈O₇), and combinations thereof. Specific examples of performing the electrochemical type of metal surface activation of this sub-step, combined with an electrochemical type of chelator binding procedure of the next sub-step, are provided in Examples 4, 9, and 11, in the Examples section hereinbelow.

Completion of the sub-step of activating (via ionizing and charging, oxidizing or reducing) metal surface (M) of medical implant component 12, results in forming an activated (ionized and charged, oxidized or reduced) metal surface (M) which includes a sub-population of exposed surface metal ions m1, m2, m4, m5, m7, m8, m10, and m11, each of which is ionized and charged (cationic or anionic) and capable of being chelated (complexed) to, and therefore, binding, one or more chelator molecules c1-c6, of chelator (C).

Binding (Via Chelation) the Chelator (C) to the Activated Metal Surface (M)

In this sub-step of the method for manufacturing the ‘metal chelated surface’ medical device, 10 or 10′ (FIGS. 1 and 2, respectively), of the present invention, there is binding (via chelation) of chelator (C) to the activated (ionized and charged) metal surface (M) of medical implant component 12, for forming medical implant component 12 having metal surface (M) to which is chelated chelator (C) in an (M)-(C) chelate type of coordination compound configuration. Accordingly, in this sub-step, there is chelating (complexing) ionized and charged (cationic or anionic) exposed surface metal ions m1, m2, m4, m5, m7, m8, m10, and m11, of the activated metal surface (M), obtained from the previous sub-step, by chelator molecules c1-c6, of chelator (C), for forming, for example, metal surface (M)-chelator (C) chelate type of coordination compound configurations m1-c1, m2-c2, m4-c3, m7-c4, m8-c5, and m10-c6, as illustrated in FIG. 1, or, alternatively, for forming, for example, metal surface (M)-chelator (C) chelate type of coordination compound configurations c1-m1-c2, c4-m7-c5, m4-c3-m5, and m10-c6-m11, as illustrated in FIG. 2.

Binding (via chelation) of chelator (C) to metal surface (M) is performed by using a suitable type of chelator binding (chelating) procedure, for example, a chemical type or an electrochemical type of chelator binding (chelating) procedure. This sub-step is performed either separate from, or together with, the previous sub-step of activating metal surface (M) of medical implant component 12.

Exemplary conditions of separately applying the chemical type of chelator binding procedure, include exposing the activated (ionized and charged, oxidized or reduced) metal surface (M) of medical implant component 12 (obtained from the previously completed metal surface activation sub-step) to a liquid phase form of a chelator compound of chelator (C), for example, an aqueous solution within which the chelator compound molar concentration is between about 0.1 M and about 1 M, at room temperature (20-25° C.), for a time period in a range of between about 30 minutes and about 180 minutes. A specific example of performing this chemical type of chelator binding procedure of this sub-step is provided in Example 3, in the Examples section hereinbelow.

Exemplary conditions of applying the electrochemical type of chelator binding procedure together with the previously described electrochemical type of metal surface activation sub-step, include the same exemplary conditions of the electrochemical oxidation type of a metal surface activation procedure, but including an amount, for example, 1 mmole, of the chelator compound of chelator (C), and including an amount, for example, 1% (vol/vol) of an alcohol, for example, ethanol, in the electrolytic fluid or bath. Specific examples of performing the electrochemical type of metal surface activation of the previous sub-step, combined with the electrochemical type of chelator binding procedure of this sub-step, are provided in Examples 4, 9, and 11, in the Examples section hereinbelow.

Reactively Combining a First Chemical Entity Specie with a Second Chemical Entity Specie, for Forming a Third (combination) Chemical Entity Specie, of Chemical Entity (X)

In this sub-step, there is reactively combining a first type of a chemical entity specie, for example, a drug, a biological moiety, or other chemical entity specie, d2 or d4, of chemical entity (X), with a second type of a chemical entity specie, for example, a linker or spacer chemical entity specie, L1 or L2, respectively, of chemical entity (X), for forming a third type of a chemical entity specie, of chemical entity (X), for example, a linker-drug or a linker-biological moiety combination chemical entity specie, L1-d2 or L2-d4, respectively, of chemical entity (X). This sub-step is performed by using any suitable prior art wet chemistry techniques and procedures for reactively combining two chemical entity species for forming a third (combination) third chemical entity specie. Three examples of performing this sub-step are provided in Examples 5, 6, and 8, in the Examples section hereinbelow.

Binding the Third (Combination) Chemical Entity (X) to the Metal Chelated (Complexed) Chelator (C)

In this sub-step, the third type of chemical entity specie of chemical entity (X), in particular, one of the linker-drug or linker-biological moiety combinations, L1-d2 or L2-d4, of chemical entity (X), obtained from the previous sub-step, is reacted with a metal chelated (complexed) chelator molecule, for example, c3 or c5, respectively, of chelator (C), that is already bound on metal surface (M), for example, in a metal surface (M)-chelator (C) chelate type of coordination compound configuration m4-c3 or m8-c5, respectively, for forming the metal surface (M)-chelator (C)-chemical entity (X) chelate type of coordination compound configuration, for example, m4-c3-L1-d2 or m8-c5-L2-d4, respectively, as illustrated in FIGS. 1 and 2. This sub-step is performed by using any suitable prior art wet chemistry techniques and procedures for reacting (binding) a chemical entity with a metal chelated (complexed) chelator. An example of performing this sub-step is provided in Example 7, in the Examples section hereinbelow.

By implementing the just described method for manufacturing a ‘metal chelated surface’ medical device, for example, ‘metal chelated surface’ medical device 10 or 10′ (FIGS. 1 and 2, respectively), of the present invention, in accordance with the above illustratively described and quantified extent or amount of surface coverage or surface concentration of a chemical or of chemicals bound to metal surface (M), then any given chemical or chemicals, for example, metal chelated (complexed) chelator molecules, for example, c2, c3, c4, or/and c5, of chelator (C), directly bound onto metal surface (M), or/and any given chemical or chemicals, for example, chemical entity species (uncharged or charged atoms or/and molecules), for example, d1, L1, d2, d3, L2, or/and d4, of chemical entity (X), bound onto metal surface (M) via chelator molecules c2, c3, c4, and c5, respectively, of chelator (C), or/and any given combination of chemicals of chelator (C) and chemical entity (X), for example, c2-d1, c3-L1, c4-d3, c5-L2, c3-L1-d2, or/and c5-L2-d4, bound onto metal surface (M) via chelator molecules c2, c3, c4, c5, c3, and c5, respectively, of chelator (C), or/and any given combination of chemicals of chemical entity (X), for example, L1-d2 or/and L2-d4, bound onto metal surface (M) via chelator molecules c3 and c5, respectively, of chelator (C), in the metal surface (M)-chelator (C)-chemical entity (X) chelate type of coordination compound configurations m2-c2-d1, m4-c3-L1, m7-c4-d3, and m8-c5-L2, or/and, in the metal surface (M)-chelator (C)-chemical entity (X) chelate type of coordination compound configurations m4-c3-L1-d2, and m8-c5-L2-d4, as illustrated in FIGS. 1 and 2, is (are) thus bound on metal surface (M), in the surface coating region in the form of a surface coating, with respect to (per) an appropriate unit of surface area of metal surface (M) of medical implant component 12, to an extent or amount of surface coverage or surface concentration greater than 100 picograms (pg) and greater than 1 picomole (pmol), respectively, of the single component or the combination of components, per square centimeter (cm²) of metal surface (M).

Accordingly, for example, in the case that medical implant component 12 represents at least a section of a metal wire, a metal filament, or a metal thread, of a stent having an external (outer or abluminal) side facing a vessel wall and an internal (inner or luminal) side facing the hollow inside or lumen of the stent, then the just illustratively described minimal or lower limit quantity or amount of surface coverage or surface concentration of a chemical or of chemicals bound to metal surface (M) corresponds to the bound chemical or chemicals being in the form of a surface coating on at least a section of a metal wire, a metal filament, or a metal thread, of an external (outer or abluminal) side or/and an internal (inner or luminal) side, of the section of the metal wire, metal filament, or metal thread, of the stent.

For illustrative purposes only, in a non-limiting manner, as an example, as shown illustrated in FIGS. 1-2, for metal surface (M) of medical implant component 12 representing an external (outer or abluminal) side of at least a section of a metal wire, a metal filament, or a metal thread, of a stent, facing a vessel wall, for example, vessel wall 52, then the just illustratively described minimal or lower limit quantity or amount of surface coverage or surface concentration of a chemical or of chemicals bound to metal surface (M) corresponds to the bound chemical or chemicals being in the form of a surface coating on an external (outer or abluminal) side of at least a section of a metal wire, a metal filament, or a metal thread, of a stent, facing a vessel wall, for example, vessel wall 52.

According to another main aspect of the present invention, there is provided a medical implant system characterized by including: (a) a medical implant component having a metal surface (M) to which is bound a chemical entity (X) via a chelator (C) chelated to the metal surface in an (M)-(C)-(X) configuration; and (b) a delivery device for delivering the medical implant component to a pre-determined position in a subject.

In the medical implant system of the present invention, the medical implant component corresponds to the medical implant component of the medical device of the present invention. Accordingly, the medical implant component, in the medical implant system, corresponds to medical implant component 12 of ‘metal chelated surface’ medical device 10 or 10′ (FIGS. 1 and 2, respectively). Moreover, the medical implant component is any of the above illustratively described embodiments or configurations of medical implant component 12 to which is bound a chemical entity (X) via a chelator (C) chelated to the metal surface (M) in an (M)-(C)-(X) configuration, of any of the above illustratively described embodiments or configurations of ‘metal chelated surface’ medical device 10 or 10′.

It is to be fully understood that all physicochemical structural and functional aspects, characteristics, and features, which were previously illustratively described for ‘metal chelated surface’ medical device 10 or 10′ (FIGS. 1 and 2, respectively), regarding the various different possible embodiments and configurations of medical implant component 12 to which is bound a chemical entity (X) via a chelator (C) chelated to the metal surface (M) in an (M)-(C)-(X) configuration, and regarding the various different possible embodiments and configurations of the metal surface (M)-chelator (C)-chemical entity (X) chelate type of coordination compound configurations, are clearly fully applicable for illustratively describing the same for medical implant component 12 in the medical implant system of the present invention.

Additionally, it is to be fully understood that all aspects which were previously illustratively described regarding the electronic states and bonding configurations of metal surface (M), chelator (C), and chemical entity (X), singly or in combination, of the exemplary embodiments of ‘metal chelated surface’ medical device 10 or 10′, and regarding either stability (that is, non-cleavable or non-breakable), or selective cleavage or breakage of the various different types of bonding or bonding-like (affinity) interaction in the metal surface (M)-chelator (C)-chemical entity (X) chelate type of coordination compound configurations, of the exemplary embodiments of ‘metal chelated surface’ medical device 10 or 10′ (FIGS. 1 and 2, respectively), are clearly fully applicable for illustratively describing the same for medical implant component 12 in the medical implant system of the present invention.

Additionally, it is to be fully understood that that all aspects which were previously illustratively described regarding the extent or amount of surface coverage by, and surface concentration of, the chemical or chemicals, chelator (C) or/and chemical entity (X), singly or in combination, in any of the metal surface (M)-chelator (C)-chemical entity (X) chelate type of coordination compound configurations, of ‘metal chelated surface’ medical device 10 or 10′ (FIGS. 1 and 2, respectively), which is (are) bound on metal surface (M) of medical implant component 12 of ‘metal chelated surface’ medical device 10 or 10′, respectively, are clearly fully applicable for illustratively describing the same for medical implant component 12 in the medical implant system of the present invention.

Additionally, it is to be fully understood that that all aspects which were previously illustratively described regarding the structure, function, and composition, of each of metal surface (M), chelator (C), and chemical entity (X), singly or in combination, and, general and specific examples of each, in any of the metal surface (M)-chelator (C)-chemical entity (X) chelate type of coordination compound configurations, of ‘metal chelated surface’ medical device 10 or 10′ (FIGS. 1 and 2, respectively), are clearly fully applicable for illustratively describing the same for medical implant component 12 in the medical implant system of the present invention.

Additionally, it is to be fully understood that all aspects which were previously illustratively described regarding the method of manufacturing an implantable medical device, for example, ‘metal chelated surface’ medical device, 10 or 10′ (FIGS. 1 and 2, respectively), of the present invention, characterized by including the step of binding to metal surface (M) of medical implant component 12, chemical entity (X), via chelator (C), in an (M)-(C)-(X) configuration, sub-steps thereof, procedures thereof, exemplary conditions thereof, and Examples 1-7 (hereinbelow) thereof, are clearly fully applicable for illustratively describing the same for medical implant component 12 in the medical implant system of the present invention.

In the medical implant system of the present invention, the medical implant component, for example, medical implant component 12, is delivered to, and implanted at, a pre-determined position in a subject, by using an appropriate delivery device, herein, generally referred to as delivery device 60, as illustrated in FIGS. 1 and 2. The specific type and use, in terms of structure and function, of delivery device 60 for delivering medical implant component 12 to a pre-determined position in a subject, and for implanting the delivered medical implant component 12, is determined by the specific type and use, in terms of structure and function, of medical implant component 12 being delivered and implanted, as well as being determined by the specific location and physiological properties and characteristics of the pre-determined position to which medical implant component 12 is to be delivered, and implanted, in the subject.

As previously illustratively described hereinabove, in ‘metal chelated surface’ medical device 10 or 10′ (FIGS. 1 and 2, respectively), medical implant component 12 generally corresponds to, and is generally representative of, at least a section of at least a part or component having a metal surface (M), of an entire or whole medical implant, such as a stent or a prosthesis. Alternatively, medical implant component 12 may also generally correspond to, and be representative of, an entire or whole part or component having a metal surface (M), of a medical implant, such as a stent or a prosthesis, or, alternatively, an entire or whole medical implant having a metal surface (M), such as an entire or whole stent having a metal surface (M), or an entire or whole prosthesis having a metal surface (M).

Accordingly, for an exemplary embodiment of the present invention, wherein medical implant component 12 generally corresponds to, and is representative of, an entire or whole medical implant having a metal surface (M), such as an entire or whole stent having a metal surface (M), or an entire or whole prosthesis having a metal surface (M), to which is bound a chemical entity (X) via a chelator (C) chelated to the metal surface (M) in an (M)-(C)-(X) configuration, then the specific type and use, in terms of structure and function, of delivery device 60 for delivering medical implant component 12, for example, in the form of an entire stent or an entire prosthesis, and for implanting the delivered medical implant component 12, to a pre-determined position in a subject, is determined by the specific type and use, in terms of structure and function, of medical implant component 12, for example, as an entire stent or an entire prosthesis, being delivered and implanted, as well as being determined by the specific location in the subject, for example, inside the cavity of a blood vessel, in the case of a stent, or, inside a socket or connection of a limb, bone, or other body part, in the case of a prosthesis, and physiological properties and characteristics of the pre-determined position, for example, inside the cavity of the blood vessel, or inside the socket or connection of the limb, bone, or other body part, to which medical implant component 12 is to be delivered, and implanted, in the subject.

For illustrative purposes only, in a non-limiting manner, as an example, as shown illustrated in FIGS. 1 and 2, for medical implant component 12 corresponding to, and being representative of, an entire or whole stent having a metal surface (M) to which is bound a chemical entity (X) via a chelator (C) chelated to the metal surface (M) in an (M)-(C)-(X) configuration, then medical implant component 12 in the form of a metal chelated surface and chemically coated stent, is to be delivered to, and implanted at, a pre-determined position in a subject, for example, inside the cavity of a blood vessel (for example, cavity 50 of a blood vessel), for being longitudinally extended along the side of the blood vessel wall, for example, blood vessel wall 52, by using a stent type of delivery device 60, in general, and by using a drug (or a biological moiety) coated or drug (or a biological moiety) eluting stent type of delivery device 60, in particular.

A stent type, or, drug coated or drug eluting stent type, of delivery device, for example, delivery device 60, usable for delivering, and implanting, medical implant component 12, for example, in the form of a metal chelated surface and chemically coated stent, to a pre-determined position in a subject, for example, inside cavity 50 of a blood vessel, is well known and taught about in the prior art.

An exemplary stent, or, drug (or a biological moiety) coated or drug (or a biological moiety) eluting stent, type of delivery device 60, is in the form of a balloon catheter. Ordinarily, an inflatable balloon, for expanding (the initially collapsed) medical implant component 12 (in the form of a metal chelated surface and chemically coated stent) following delivery of medical implant component 12 to the pre-determined position in the subject, inside cavity 50 of a blood vessel, is located on the distal end of the balloon catheter type of delivery device 60, around which is positioned the initially collapsed medical implant component 12.

Upon positioning the non-inflated balloon of the balloon catheter type of delivery device 60 to the pre-determined position in the subject, the non-inflated balloon is inflated, thereby causing medical implant component 12 (in the form of a metal chelated surface and chemically coated stent) to radially expand outward toward blood vessel wall 52. Then, medical implant component 12 is set at the desired functional expanded state inside cavity 50 of the blood vessel. Subsequent deflation of the balloon and extraction of the balloon catheter type of delivery device 60, leaves the expanded medical implant component 12 (metal chelated surface and chemically coated stent) at the pre-determined position in the subject, inside cavity 50 of the blood vessel and longitudinally extended along the side of blood vessel wall 52. In addition to expanded and implanted medical implant component 12 structured and functioning as a stent for performing its main function of stenting or supporting blood vessel wall 52, the activity of bound chemical entity (X), for example, a drug or a biological moiety, or, a linker or spacer to which a drug or a biological moiety is bound, exhibits an efficacy for preventing or/and treating a medical condition, such as a cardiovascular type of medical condition, for example, restenosis, in general, and in-stent restenosis, in particular, or/and thrombosis, in the subject.

Regarding a balloon catheter type of delivery device 60, for example, each of the prior art CYPHER and TAXUS drug eluting stents, previously described in the Background section, is ordinarily delivered to, and implanted at, a pre-determined position in a subject, inside a blood vessel, using a balloon catheter type of delivery device. For the CYPHER drug eluting stent (Cordis/Johnson & Johnson, U.S. Pat. Nos. 6,585,764; 6,273,913), stent delivery can be performed using, for example, either the Raptor Rail rapid delivery system (device) with a usable or working length of 137 centimeters, or, the Over-the-Wire delivery system (device) with a usable or working length of 145 centimeters. Each delivery system (device) involves use of a single-layer nylon balloon, approximately 2 mm longer than the stent itself. Nominal pressure is rated at 11 atm, while burst pressure is approximately 16 atm. For the TAXUS drug eluting stent (Boston Scientific, U.S. Pat. Nos. 6,344,028; 6,197,051; 6,179,817), stent delivery is performed using, for example, either the Monorail stent delivery system (device) with a usable or working length of 140 cm, or, the Over-the-Wire stent delivery system (device) with a usable or working length of 135 cm. Each delivery system (device) involves use of a balloon with a rated nominal pressure of 9 atm and a rated burst pressure of 18 atm.

In a non-limiting manner, any of the just described CYPHER or TAXUS drug eluting stent type of delivery systems (devices), or any other prior art stent type, or, drug coated or drug eluting stent type, of delivery device, is suitable for implementing the present invention, as delivery device 60, for delivering medical implant component 12, for example, in the form of a metal chelated surface and chemically coated stent, to a pre-determined position in a subject, for example, inside cavity 50 of a blood vessel, for being longitudinally extended along the side of the blood vessel wall, for example, blood vessel wall 52.

According to another main aspect of the present invention, there is provided a method of implanting a medical device characterized by including the step of implanting in a subject in need thereof a medical device which includes a medical implant having a metal surface (M) to which is bound a chemical entity (X) via a chelator (C) chelated to the metal surface in an (M)-(C)-(X) configuration. Accordingly, in the present invention, there is provided a method of implanting a medical device characterized by including the step of implanting in a subject in need thereof a medical device, for example, ‘metal chelated surface’ medical device 10 or 10′ (FIGS. 1 and 2, respectively), which includes a medical implant component, in particular, medical implant component 12, having a metal surface (M) to which is bound a chemical entity (X) via a chelator (C) chelated to the metal surface (M) in an (M)-(C)-(X) configuration.

In general, there is a wide variety of different possible steps, sub-steps thereof, and, sequences and orders thereof, for performing the method of implanting a medical device characterized by including the step of implanting in a subject in need thereof ‘metal chelated surface’ medical device 10 or 10′, which includes medical implant component 12 having a metal surface (M) to which is bound a chemical entity (X) via a chelator (C) chelated to the metal surface (M) in an (M)-(C)-(X) configuration. For the objective of illustrating implementation of the present invention, in a non-limiting manner, the method of implanting ‘metal chelated surface’ medical device 10 or 10′ in a subject in need thereof, is performed in full accordance with the immediately preceding illustratively described implementation of the medical implant system of the present invention, characterized by including: (a) medical implant component 12 of ‘metal chelated surface’ medical device 10 or 10′ having a metal surface (M) to which is bound a chemical entity (X) via a chelator (C) chelated to the metal surface in an (M)-(C)-(X) configuration; and (b) delivery device 60 for delivering medical implant component 12 to a pre-determined position in a subject.

Accordingly, for illustrative purposes only, in a non-limiting manner, as an example, as shown illustrated in FIGS. 1 and 2, for medical implant component 12 of ‘metal chelated surface’ medical device 10 or 10′, respectively, corresponding to, and being representative of, an entire or whole stent having a metal surface (M) to which is bound a chemical entity (X) via a chelator (C) chelated to the metal surface (M) in an (M)-(C)-(X) configuration, then ‘metal chelated surface’ medical device 10 or 10′, including medical implant component 12 in the form of a metal chelated surface and chemically coated stent, is delivered to, and implanted at, a pre-determined position in a subject, for example, inside the cavity of a blood vessel (for example, cavity 50 of a blood vessel), for being longitudinally extended along the side of the blood vessel wall, for example, blood vessel wall 52, by using a stent type of delivery device 60, in general, and by using a drug (or a biological moiety) coated or drug (or a biological moiety) eluting stent type of delivery device 60, in particular, for example, in the form of a balloon catheter. In addition to expanded and implanted medical implant component 12 structured and functioning as a stent for performing its main function of stenting or supporting blood vessel wall 52, the activity of bound chemical entity (X), for example, a drug or a biological moiety, or, a linker or spacer to which a drug or a biological moiety is bound, exhibits an efficacy for preventing or/and treating a medical condition, such as a cardiovascular type of medical condition, for example, restenosis, in general, and in-stent restenosis, in particular, or/and thrombosis, in the subject.

According to another aspect of the present invention, there is provided a method of implanting a medical device characterized by including a step of implanting in a subject in need thereof a medical device which includes a medical implant component having a surface to which is bound a chemical at a surface concentration of greater than 100 picograms (pg) per cm².

By implementing the just described method of implanting a medical device characterized by including the step of implanting in a subject in need thereof ‘metal chelated surface’ medical device 10 or 10′ (FIGS. 1 and 2, respectively), of the present invention, in accordance with the above illustratively described and quantified extent or amount of surface coverage or surface concentration of a chemical or of chemicals bound to metal surface (M), then any given chemical or chemicals, for example, metal chelated (complexed) chelator molecules, for example, c2, c3, c4, or/and c5, of chelator (C), directly bound onto metal surface (M), or/and any given chemical or chemicals, for example, chemical entity species (uncharged or charged atoms or/and molecules), for example, d1, L1, d2, d3, L2, or/and d4, of chemical entity (X), bound onto metal surface (M) via chelator molecules c2, c3, c4, and c5, respectively, of chelator (C), or/and any given combination of chemicals of chelator (C) and chemical entity (X), for example, c2-d1, c3-L1, c4-d3, c5-L2, c3-L1-d2, or/and c5-L2-d4, bound onto metal surface (M) via chelator molecules c2, c3, c4, c5, c3, and c5, respectively, of chelator (C), or/and any given combination of chemicals of chemical entity (X), for example, L1-d2 or/and L2-d4, bound onto metal surface (M) via chelator molecules c3 and c5, respectively, of chelator (C), in the metal surface (M)-chelator (C)-chemical entity (X) chelate type of coordination compound configurations m2-c2-d1, m4-c3-L1, m7-c4-d3, and m8-c5-L2, or/and, in the metal surface (M)-chelator (C)-chemical entity (X) chelate type of coordination compound configurations m4-c3-L1-d2, and m8-c5-L2-d4, as illustrated in FIGS. 1 and 2, is (are) thus bound on metal surface (M), in the surface coating region in the form of a surface coating, with respect to (per) an appropriate unit of surface area of metal surface (M) of medical implant component 12, to an extent or amount of surface coverage or surface concentration greater than 100 picograms (pg) and greater than 1 picomole (pmol), respectively, of the single component or the combination of components, per square centimeter (cm²) of metal surface (M).

Thus, in accordance with another main aspect of the present invention, there is provision of a method of implanting a medical device characterized by including the step of implanting in a subject in need thereof a medical device, in particular, ‘metal chelated surface’ medical device 10 or 10′ (FIGS. 1 and 2, respectively), which includes medical implant component 12 having a surface, for example, a metal surface, in particular, metal surface (M), to which is bound a chemical at a surface concentration of greater than 100 picograms (pg) per cm².

According to another main aspect of the present invention, there is provided a method of preventing or/and treating a medical condition of a subject characterized by including the step of implanting in the subject a medical device which includes a medical implant having a metal surface (M) to which is bound a chemical entity (X) via a chelator (C) chelated to the metal surface in an (M)-(C)-(X) configuration, such that activity of the bound chemical entity exhibits an efficacy for preventing or/and treating the medical condition. Accordingly, in the present invention, there is provided a method of preventing or/and treating a medical condition of a subject characterized by including the step of implanting in the subject a medical device, for example, ‘metal chelated surface’ medical device 10 or 10′ (FIGS. 1 and 2, respectively), which includes a medical implant component, in particular, medical implant component 12, having a metal surface (M) to which is bound a chemical entity (X) via a chelator (C) chelated to the metal surface (M) in an (M)-(C)-(X) configuration, such that activity of the bound chemical entity (X) exhibits an efficacy for preventing or/and treating the medical condition.

In addition to implanted medical implant component 12 of ‘metal chelated surface’ medical device 10 or 10′ (FIGS. 1 and 2, respectively) structured and functioning, for example, as an implanted stent for performing its main function of stenting or supporting blood vessel wall 52, or as an implanted prosthesis for performing its main function of replacing, supplementing, or supporting, a limb, bone, or other body part, the activity of bound chemical entity (X), for example, a drug, or, a linker or spacer to which a drug is bound, exhibits an efficacy for preventing or/and treating a medical condition.

For the objective of illustrating implementation of the present invention, in a non-limiting manner, in the method of preventing or/and treating a medical condition of a subject, of the present invention, the step of implanting in the subject a medical device, for example, ‘metal chelated surface’ medical device 10 or 10′ (FIGS. 1 and 2, respectively), which includes a medical implant component, in particular, medical implant component 12, having a metal surface (M) to which is bound a chemical entity (X) via a chelator (C) chelated to the metal surface (M) in an (M)-(C)-(X) configuration, is performed in full accordance with the preceding illustratively described implementation of the medical implant system of the present invention, characterized by including: (a) medical implant component 12 of ‘metal chelated surface’ medical device 10 or 10′ having a metal surface (M) to which is bound a chemical entity (X) via a chelator (C) chelated to the metal surface in an (M)-(C)-(X) configuration; and (b) delivery device 60 for delivering medical implant component 12 to a pre-determined position in a subject.

Accordingly, for illustrative purposes only, in a non-limiting manner, as an example, as shown illustrated in FIGS. 1 and 2, for medical implant component 12 of ‘metal chelated surface’ medical device 10 or 10′, respectively, corresponding to, and being representative of, an entire or whole stent having a metal surface (M) to which is bound a chemical entity (X) via a chelator (C) chelated to the metal surface (M) in an (M)-(C)-(X) configuration, then ‘metal chelated surface’ medical device 10 or 10′, including medical implant component 12 in the form of a metal chelated surface and chemically coated stent, is delivered to, and implanted at, a pre-determined position in a subject, for example, inside the cavity of a blood vessel (for example, cavity 50 of a blood vessel), for being longitudinally extended along the side of the blood vessel wall, for example, blood vessel wall 52, by using a stent type of delivery device 60, in general, and by using a drug (or a biological moiety) coated or drug (or a biological moiety) eluting stent type of delivery device 60, in particular, for example, in the form of a balloon catheter.

In addition to expanded and implanted medical implant component 12 structured and functioning as a stent for performing its main function of stenting or supporting blood vessel wall 52, the activity of bound chemical entity (X), for example, a drug, or, a linker or spacer to which a drug is bound, exhibits an efficacy for preventing or/and treating a medical condition, such as a cardiovascular type of medical condition, for example, restenosis, in general, and in-stent restenosis, in particular, or/and thrombosis, in the subject.

For example, for each metal surface (M)-chelator (C)-chemical entity (X) chelate type of coordination compound configuration, m2-c2-X (wherein X=d1), and m7-c4-X (wherein X=d3), of ‘metal chelated surface’ medical device 10 or 10′ (FIGS. 1 and 2, respectively) implanted in cavity 50 of the blood vessel, the bonding, or the at least bonding-like (affinity) interaction, between the corresponding metal chelated (complexed) chelator molecule c2 and c4, respectively, of chelator (C), and the corresponding chemical entity specie d1 and d3, respectively, of chemical entity (X), is either stable (not ordinarily cleavable or breakable) in cavity 50 of the blood vessel, or is selectively cleavable or breakable via bond or bond-like cleaving or breaking mechanism 30 and 32, respectively, and an appropriately corresponding bond or bond-like cleaving or breaking agent v1 and v2, respectively, as illustrated in FIGS. 1 and 2. Such bond or bond-like cleavage or breakage results in separation, release or elution, and subsequent migration, of the corresponding chemical entity specie d1 and d3, respectively, of chemical entity (X), away from the corresponding metal chelated (complexed) chelator molecule c2 and c4, respectively, of chelator (C), through cavity 50 and towards blood vessel wall 52 of the blood vessel, as illustrated in FIG. 3.

For example, for each metal surface (M)-chelator (C)-chemical entity (X) chelate type of coordination compound configuration, m4-c3-X (wherein X=L1-d2), and m8-c5-X (wherein X=L2-d4), of ‘metal chelated surface’ medical device 10 or 10′ (FIGS. 1 and 2, respectively) implanted in cavity 50 of the blood vessel, the bonding, or the at least bonding-like (affinity) interaction, between the corresponding (chelator bonded or interacting) chemical entity specie L1 and L2, respectively, and the corresponding additional chemical entity specie d2 and d4, respectively, of chemical entity (X), is either stable (not ordinarily cleavable or breakable) in cavity 50 of the blood vessel, or is selectively cleavable or breakable via bond or bond-like cleaving or breaking mechanism 34 and 36, respectively, and an appropriately corresponding bond or bond-like cleaving or breaking agent v3 and v4, respectively, as illustrated in FIGS. 1 and 2. Such bond or bond-like cleavage or breakage results in separation, release or elution, and subsequent migration, of the corresponding additional chemical entity specie d2 and d4, respectively, away from the corresponding (chelator bonded or interacting) chemical entity specie L1 and L2, respectively, of chemical entity (X), through cavity 50 and towards blood vessel wall 52 of the blood vessel, as illustrated in FIG. 3.

Alternatively, for example, in each metal surface (M)-chelator (C)-chemical entity (X) chelate type of coordination compound configuration, m4-c3-L1-d2, and m8-c5-L2-d4, of ‘metal chelated surface’ medical device 10 or 10′ (FIGS. 1 and 2, respectively) implanted in cavity 50 of the blood vessel, the bonding, or the at least bonding-like (affinity) interaction, between the corresponding metal chelated (complexed) chelator molecule c3 and c5, respectively, of chelator (C), and the corresponding chemical entity specie L1 and L2, respectively, of chemical entity (X), is either stable (not ordinarily cleavable or breakable) in cavity 50 of the blood vessel, or is selectively cleavable or breakable via bond or bond-like cleaving or breaking mechanism 38 and 40, respectively, and an appropriately corresponding bond or bond-like cleaving or breaking agent v5 and v6, respectively, as illustrated in FIGS. 1 and 2. Such bond or bond-like cleavage or breakage results in separation, release or elution, and subsequent migration, of the corresponding chemical entity specie L1-d2 and L2-d4, respectively, of chemical entity (X), away from the corresponding metal chelated (complexed) chelator molecule c3 and c5, respectively, of chelator (C), through cavity 50 and towards blood vessel wall 52 of the blood vessel.

It is therefore clearly understood that in each of the above illustratively described examples of various possible embodiments of metal surface (M)-chelator (C)-chemical entity (X) chelate type of coordination compound configurations, m2-c2-X (wherein X=d1), m7-c4-X (wherein X=d3), m4-c3-X (wherein X=L1-d2), and m8-c5-X (wherein X=L2-d4), of ‘metal chelated surface’ medical device 10 or 10′ (FIGS. 1 and 2, respectively) implanted in cavity 50 of the blood vessel, any given chemical or chemicals, for example, chemical entity species (uncharged or charged atoms or/and molecules), for example, d1, L1, d2, d3, L2, or/and d4, of chemical entity (X), bound onto metal surface (M) via chelator molecules c2, c3, c4, and c5, respectively, of chelator (C), or/and any given combination of chemical entity species of chemical entity (X), for example, L1-d2 or/and L2-d4, bound onto metal surface (M) via chelator molecules c3 and c5, respectively, of chelator (C), is (are) thus bound on metal surface (M), in the surface coating region in the form of a surface coating, and is (are) either stable (not ordinarily cleavable or breakable) in cavity 50 of the blood vessel, or is (are) selectively cleavable or breakable via a bond or bond-like cleaving or breaking mechanism 30, 32, 34, 36, 38, or 40, and an appropriately corresponding bond or bond-like cleaving or breaking agent v1, v2, v3, v4, v5, and v6, respectively, resulting in separation, release or elution, and subsequent migration, of the corresponding chemical entity specie or combination of chemical entity species of chemical entity (X), away from the corresponding metal chelated (complexed) chelator molecule c2 and c4, respectively, of chelator (C), or away from the corresponding (chelator bonded or interacting) chemical entity specie L1 and L2, respectively, of chemical entity (X), or away from the corresponding metal chelated (complexed) chelator molecule c3 and c5, respectively, of chelator (C), through cavity 50 and towards blood vessel wall 52 of the blood vessel.

Moreover, for an exemplary preferred embodiment of the present invention wherein a given chemical entity specie, in particular, chemical entity specie d1, d2, d3, or d4, of chemical entity (X), is a drug (uncharged or charged molecule) or a biological moiety (uncharged or charged molecule), or/and wherein a given other chemical entity specie, in particular, chemical entity specie L1 or L2, of chemical entity (X), is a linker or spacer (uncharged or charged atom or molecule), bonded to, or at least bond-like (affinity) interacting with, a drug or a biological moiety type of chemical entity specie, for example, chemical entity specie d2 or d4, respectively, of chemical entity (X), then any of the above indicated chemical entity species, singly or in combination, of chemical entity (X), as part of either the stable (non-cleavable or non-breakable) type of bonding or the at least bonding-like (affinity) interaction configuration, or, as part of the selectively cleavable or breakable type of bonding or the at least bonding-like (affinity) interaction configuration, potentially has a therapeutic activity directed or located within cavity 50 or/and at blood vessel wall 52 of the blood vessel, which exhibits an efficacy for preventing or/and treating a medical condition in the subject.

An exemplary type of drug or a biological moiety, functioning as one or more chemical entity species d1, d2, d3, or/and d4, of chemical entity (X), which is suitable for implementing the present invention, is a drug or a biological moiety used for preventing or/and treating a medical condition, such as a cardiovascular type of medical condition, of a subject. Exemplary cardiovascular types of a medical condition of a subject are restenosis, in general, and in-stent restenosis, in particular, and thrombosis. Accordingly, an exemplary type of drug used for preventing or/and treating a cardiovascular type of medical condition of a subject is a cardiovascular drug. An exemplary type of cardiovascular drug is a drug that prevents or/and inhibits onset or/and progression of restenosis, in general, and in-stent restenosis, in particular. Another exemplary type of cardiovascular drug is a drug that prevents or/and inhibits onset or/and progression of thrombosis.

Numerous specific examples, of the composition of metal surface (M) of medical implant component 12, of chelator molecules or compounds of chelator (C), of drug or biological moiety types of chemical entity species of chemical entity (X), and of linker or spacer types of chemical entity species of chemical entity (X), for the above illustratively described exemplary embodiments of metal surface (M)-chelator (C)-chemical entity (X) chelate type of coordination compound configurations, m2-c2-X (wherein X=d1), m7-c4-X (wherein X=d3), m4-c3-X (wherein X=L1-d2), and m8-c5-X (wherein X=L2-d4), of ‘metal chelated surface’ medical device 10 or 10′ (FIGS. 1-3) implanted in cavity 50 of the blood vessel, which are well suitable for implementing the present invention, are clearly listed hereinabove.

Above illustratively described novel and inventive aspects and characteristics, and advantages thereof, of the present invention further become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

Reference is now made to the following examples, Examples 1-11, which together with the above description, illustrate the invention in a non-limiting fashion.

### EXAMPLE 1

### Removing Metal Surface Blocking from a Metal Surface (M)

Metal surface blocking, in the form of phosphoric acid and sulfuric acid ions, was removed from the metal surface of an electropolished metal stent made of 316L stainless steel wiring 0.2 mm thick.

An electropolished metal stent made of 316L stainless steel wiring 0.2 mm thick, was subjected to surface examination using an SEM (scanning electron microscope) and elemental analysis of selected elements using a spectrometer. The stent was found to have a smooth surface with some wrinkled and pitted areas. Elemental analysis of selected elements of the stent was as follows: Cr (17.8%), Ni (14.6%), Mo (2.8%), Mn (2.4%), and Si (0.2%), which conformed well to the same elemental analysis of a standard 316L stainless steel foil 0.2 mm thick.

The stent was exposed to a dilute aqueous solution of ammonium hydroxide (NH₄OH) having a concentration in a range of between about 5% and about 30% (vol/vol), at room temperature (20-25° C.), for about 30 minutes, followed by fully washing the NH₄OH treated stent for five times with water. Small white crystals, indicative of ammonium phosphate and ammonium sulfate salts, resulting from reaction between the ammonium hydroxide (NH₄OH) and the phosphoric acid and sulfuric acid ions, appeared on the surface of the stent. These salts were fully dissolved and washed off the surface of the stent using water.

### EXAMPLE 2

### Activating (Via Ionizing and Charging) a Metal Surface (M)

The metal surface of a stainless steel stent, absent of metal surface blocking, was activated by using a chemical oxidation type of a metal surface activation procedure.

An oxidizing reagent, 36 mg of ammonium persulfate ((NH₄)₂S₂O₈), was dissolved in 2 ml of a 10% solution of NaOH in water. The stainless steel stent (whose metal surface blocking was removed as described in Example 1) was exposed to this solution at a temperature between about 70° C. and about 100° C., for about 20 minutes. A visually noticeable different color (yellowish) appeared on the metal surface of the stent. The change in color indicated creation of activated charged metal ions, such as: [Fe⁺²O⁻], [Cr⁺³O⁻], [Ni⁺²O⁻], and [Cu⁺²O⁻], on the metal surface of the stent. This charging enabled the metal surface of the stent to be chelated to, and bind, chelator molecules of a chelator, via activated (ionized and charged, oxidized) metal ions having various possible coordination numbers, for example, 4 and 6. The activated (ionized and charged, oxidized) stent was washed several times with water and then dried.

### EXAMPLE 3

### Chemical Binding (Via Chelation) a Chelator (C) to an Activated Metal Surface (M)

In a chemical type of chelator binding procedure, a chelator was chemically bound (via chelation) to an activated (ionized and charged, oxidized) stainless steel stent, for forming a stainless steel stent having a metal surface chelated to the chelator in a metal surface—chelator chelate type of coordination compound configuration.

The activated (ionized and charged, oxidized) stainless steel stent (from Example 2) was exposed to an aqueous solution of edetic acid (EDTA) chelator having a molar concentration of 0.1 M, and including 0.1 M of oxalic acid, at room temperature (20-25° C.), for a time period of between about 30 minutes and about 180 minutes. Following the chemical binding procedure, the edetic acid (EDTA) chelator bound stainless steel stent was fully washed with water and then dried.

### EXAMPLE 4

### Combined Activating (Via Ionizing and Charging) a Metal Surface (M) and Electrochemical Binding (Via Chelation) a Chelator (C) to the Activated Metal Surface

In a combined metal surface activating and chelator binding (chelating) procedure, the metal surface of a stainless steel stent (whose metal surface blocking was removed as described in Example 1), absent of metal surface blocking, was activated by using an electrochemical oxidation type of metal surface activation procedure, following which a chelator was bound (via chelation) to the activated metal surface by using an electrochemical type of chelator binding (chelating) procedure, where both procedures were performed at the same time using the same electrochemical cell.

The stent was immersed into an electrochemical cell which housed an aqueous electrolytic fluid or bath having in it H₂C₂O₄, 0.5 M, and H₂SO₄, 0.5 M, as the chemical oxidants (oxidizing reagents), and 5-amino-8-hydroxyquinoline, 1 mmole, as the chelator, and including ethanol, 1% (vol/vol). The stent was conductively attached or connected to the anode electrode terminal (for oxidation), with a non-corrosive metallic element conductively attached or connected to the corresponding cathode electrode terminal, with the ratio of cathode surface area and anode surface area being about two to one. Current density of about 0.5 amps per square inch was maintained between the stent and the cathode during the electrolysis procedure, which was performed for 15 minutes at a temperature of 30° C. Following the electrolysis procedure, the 5-amino-8-hydroxyquinoline chelator bound stainless steel stent was fully washed in an ethanol/water, 1/1 (vol/vol), solution, and dried.

### EXAMPLE 5

### Reactively Combining a First Chemical Entity Specie with a Second Chemical Entity Specie, for Forming a Third (combination) Chemical Entity Specie

A first type of a chemical entity specie, rhodamine (synthetic red to pink dye), as an exemplary pseudo drug or pseudo biological moiety chemical entity specie (which upon reaction is visually detectable by the naked eye), was reactively combined with a second type of a chemical entity specie, a peptide, as an exemplary linker or spacer chemical entity specie, for forming a third type of a chemical entity specie, a peptide-rhodamine combination chemical entity specie, as an exemplary linker-pseudo drug or linker-pseudo biological moiety chemical entity specie.

An amount, 1 mmole, of rhodamine was dissolved in 20 ml of DMF (dimethyl formamide). The solution was cooled to 0° C., and then activated with 1.1 mmole of DCC (N,N′-dicyclohexylcarbodiimide) for 30 minutes on ice at 0° C., and continued for another 2 hours at room temperature. White precipitate crystals which appeared in the solution were filtered out.

An amount, 1 mmole, of dry peptide (Pro-Arg-Ser-Leu-Thr; synthesized according to the procedure herein described immediately following) was added to the solution containing the activated rhodamine dye. The reaction was carried out at room temperature for 20 hours. The reaction mixture was filtered, and the product was precipitated using diethyl ether. The product was washed several times with ether. The peptide-rhodamine (linker-pseudo drug or linker-pseudo biological moiety) chemical entity specie was separated from free rhodamine using size exclusion liquid chromatography (SEC).

The dry peptide (Pro-Arg-Ser-Leu-Thr), used in this example was synthesized according to the following procedure.

2-chlorotrityl chloride resin (100-200 mesh, 1% DVB), substitution of 1.2 g/mol, was swollen in DCM (dichloromethane) for 1 hour. The resin was washed several times with DCM. 2.4 mmole of Fmoc-Thr(tBu)-OH was dissolved in 20 ml of DCM and added to the resin. 4.8 mmole of diethyl-isopropylamine was added to the reaction. The reaction was carried out for 2 hours at room temperature. The resin was washed several times with DCM, methanol, DCM. Fmoc protecting group was removed by using a solution of 20% piperidine in DMF (dimethyl formamide) for 5 times, 5 minutes each time. The resin was washed several times with DMF, DCM, DMF.

2.4 mmole of Fmoc-Leu-OH were dissolved in DMF, with 2.4 mmole DIC (diisopropyl carbodiimide). The reaction was carried out for 2 hours at room temperature. The resin was washed several times with DMF, DCM, DMF. Fmoc protecting group was removed by incubation in 20% piperidine in DMF 3 times for 10 minutes each time. Further coupling and deprotection of Fmoc-Ser(tBu)-OH, Fmoc-Arg(Pbf)-OH, Fmoc-Pro-OH, were carried out as for Fmoc-Leu-OH.

At the end of peptide synthesis the resin was washed several times with DMF, DCM. The resin was dried in vacuum. The product was cleaved from the resin by using 1 ml of 1% of TFA (trifluoroacetate) in DCM+1% water for 30 minutes. The protecting groups were removed by using 95% of TFA with water and 2.5% of triisopropylsilane. The acid was evaporated in vacuum over KOH pellets. The final product was washed several times with diethyl-ether and dried. The dried peptide was dissolved in water and lyophilized.

### EXAMPLE 6

### Reactively Combining a First Chemical Entity Specie with a Second Chemical Entity Specie, for Forming a Third (combination) Chemical Entity Specie

A first type of a chemical entity specie, methotrexate, as an exemplary drug (or biological moiety) chemical entity specie, was reactively combined with a second type of a chemical entity specie, a peptide (Pro-Arg-Ser-Leu-Thr; synthesized according to the procedure herein described in Example 5), as an exemplary linker or spacer chemical entity specie, for forming a third type of a chemical entity specie, a peptide-methotrexate (combination) chemical entity specie, as an exemplary linker-drug (or linker-biological moiety) chemical entity specie.

An amount, 1 mmole, of methotrexate was dissolved in 10 ml of N-methyl-pyrrolidone. The solution was cooled to 0° C., and then activated with 1.1 mmole of DCC (N,N′-dicyclohexylcarbodiimide) for 30 minutes on ice at 0° C., and continued for another 2 hours at room temperature. White precipitate crystals which appeared in the solution were filtered out.

An amount, 1 mmole, of dry peptide (Pro-Arg-Ser-Leu-Thr; synthesized according to the procedure herein described immediately following) was added to the solution containing the activated methotrexate drug. The reaction was carried out at room temperature for 20 hours. The reaction mixture was filtered, and the product was precipitated using diethyl ether. The product was washed several times with ether. The peptide-methotrexate (linker-drug or linker-biological moiety) chemical entity specie was separated from free methotrexate using size exclusion liquid chromatography (SEC).

### EXAMPLE 7

### Binding the Third (combination) Chemical Entity (X) to the Metal Chelated (complexed) Chelator (C)

An amount, 155 mg of EDC (1-ethyl-3-(3′-dimethylaminopropyl)carbodiimide) was dissolved in 0.2 ml DMSO (dimethylsulfoxide), and the mixture was further diluted with water to 2 ml to achieve a final concentration of 0.5 M EDC. EDC is a water soluble carbodiimide that conjugates carboxylic and amino groups to create an amide bond.

The 5-amino-8-hydroxyquinoline chelator bound stainless steel stent (from Example 4) was exposed to the 0.5 M EDC solution, by agitation for 30 minutes at room temperature. An amount, 5 mg of the peptide-rhodamine (linker-pseudo drug or linker-pseudo biological moiety) chemical entity species (prepared as described in Example 5) was added to the EDC treated chelator bound stent and allowed to mix with agitation for one hour. The stainless steel stent whose metal surface was bound and coated with the peptide-rhodamine (linker-pseudo drug or linker-pseudo biological moiety) chemical entity species via the 5-amino-8-hydroxyquinoline chelator was washed three times with water and dried in air. Due to the presence of the rhodamine dye in the 5-amino-8-hydroxyquinoline-peptide-rhodamine coating on the stent, the stent was visually detectable by the naked eye as being of a red to pink color.

### EXAMPLE 8

### Reactively Combining a First Chemical Entity Specie with a Second Chemical Entity Specie, for Forming a Third (combination) Chemical Entity Specie, which is Reactively Combined with a Fourth Chemical Entity Specie, for Forming a Fifth (Combination) Chemical Entity Specie (a Chelate Type of Coordination Compound, (C)-(Y))

A first type of a chemical entity specie, doxorubicin, as an exemplary drug (or biological moiety) chemical entity specie, was reactively combined with a second type of a chemical entity specie, an amino acid (lysine), as an exemplary linker or spacer chemical entity specie, for forming a third type of a chemical entity specie, lysine-doxorubicin, as an exemplary linker-drug (or linker-biological moiety) (combination) chemical entity specie. The lysine-doxorubicin (combination) chemical entity specie was then reactively combined with a fourth type of a chemical entity specie, EDTA (as an exemplary chelator), for forming a fifth type of a chemical entity specie, EDTA-lysine-doxorubicin, being an exemplary chelate type of coordination compound.

The EDTA-lysine-doxorubicin (combination) chemical entity specie is a sub-group (ii) type of chelator (C)-chemical entity (X=Y=linker-drug, or linker-biological moiety) chelate type of coordination compound configuration, and is characterized by the structure of general formula: (C)-(X=Y), wherein (C) is the EDTA chelator, and (X=Y) is the lysine-doxorubicin linker-drug (or linker-biological moiety) (combination) chemical entity specie.

An amount, 1 mmole, of Fmoc-Lysine(Fmoc)-OH was dissolved in 10 ml of N-methyl-pyrrolidone, and activated with 1.1 mmole of DIC (diisopropylcarbodiimide) for 30 minutes at room temperature. An amount, 1 mmole, of dry doxorubicin was added to the solution containing the activated Fmoc-lysin(Fmoc)-OH amino acid. The reaction was carried out at room temperature for 20 hours. The reaction mixture was filtered, and the product was precipitated, and then washed with hexane. The Fmoc protecting group was removed from Fmoc-lysine(Fmoc)-doxorubicin using a solution of 20% piperidine in DCM (dichloromethane), and the lysine-doxorubicin product was precipitated using ether.

An amount, 1 mmole, of EDTA was dissolved in 10 ml of water, and activated with 1.1 mmole of EDC for 3 minutes at room temperature. Lysine-doxorubicin, 1 mmole, was added to the solution containing the activated EDTA, and the reaction was carried out at room temperature for 20 hours. The EDTA-lysine-doxorubicin product was separated from the reaction mixture by liquid chromatography.

### EXAMPLE 9

### Combined Activating (Via Ionizing and Charging) a Metal Surface (M) and Electrochemical Binding (Via Chelation) a Chelator-Linker-Drug (or Biological Moiety) (combination) Chemical Entity Specie (a Chelate Type of Coordination Compound, (C)-(Y)), to the Activated Metal Surface

In a combined metal surface activating and chelator binding (chelating) procedure, the metal surface of a stainless steel stent (whose metal surface blocking was removed as described in Example 1), absent of metal surface blocking, was activated by using an electrochemical oxidation type of metal surface activation procedure, following which an exemplary chelator-linker-drug (or chelator-linker-biological moiety) (combination) chemical entity specie (a chelate type of coordination compound, (C)-(Y)), was bound (via chelation) to the activated metal surface by using an electrochemical type of chelator binding (chelating) procedure.

The stainless steel stent (from Example 1) was immersed into an electrochemical cell which housed an aqueous electrolytic fluid having 1 mmol of the EDTA-lysine-doxorubicin (combination) chemical entity specie (chelate type of coordination compound), (prepared as described in Example 8), at acidic conditions (pH of 2-3). The stent was conductively attached or connected to the anode electrode terminal (for oxidation), with a non-corrosive metallic element conductively attached or connected to the corresponding cathode electrode terminal, with the ratio of cathode surface area and anode surface area being about two to one. Current density of about 0.5 amps per square inch was maintained between the stent and the cathode during the electrolysis procedure, which was performed for 15 minutes at a temperature of 30° C. Following the electrolysis procedure, the free EDTA-lysine-doxorubicin that was not bound to stainless steel stent was fully washed in an acid water, and dried.

### EXAMPLE 10

### Synthesis of, and Reactively Combing, a Peptide Chelator (C), with a Biological Moiety Type Chemical Entity Specie of Chemical Entity (X), for Forming a Chelator-Biological Moiety (Combination) Chemical Entity Specie (a Chelate Type of Coordination Compound, (C)-(Y))

A first type of a chemical entity specie, a peptide, as an exemplary chelator, was synthesized by solid phase peptide synthesis and then reactively combined with a second type of a chemical entity specie, a protein, as an exemplary biological moiety chemical entity specie, for forming a third type of a chemical entity specie, a peptide-protein, as an exemplary chelator-biological moiety (combination) chemical entity specie, being an exemplary chelate type of coordination compound.

The peptide chelator, poly-histidine (or poly-His peptide) (His-His-His-His-His-His), was synthesized according to the following procedure:

2-chlorotrityl chloride resin (100-200 mesh, 1% DVB), substitution of 1.2 g per mole, was swollen in DCM (dichloromethane) for 1 hour. The resin washed several times with DCM. 2.4 mmole of Fmoc-His(Trt)-OH were dissolved in 20 ml of DCM and added to the resin. 4.8 mmole of diethyl-isopropylamine were added to the reaction mixture. The reaction was carried out for 2 hours at room temperature. The resin was washed several times with DCM, methanol, DCM. Fmoc protecting group was removed by using a solution of 20% piperidine in DMF (dimethyl formamide) for 5 times, 5 minutes each time. The resin was washed several times with DMF, DCM, DMF.

2.4 mmole of Fmoc-His(Trt)-OH were dissolved in DMF, with 2.4 mmole DIC (diisopropyl carbodiimide). The reaction was carried out for 2 hours at room temperature. The resin was washed several times with DMF, DCM, DMF. Fmoc protecting group was removed by incubation in 20% piperidine in DMF for 3 times, 10 minutes each time. Further coupling and deprotection of Fmoc-His(Trt)-OH were carried out in the same manner.

At the end of the poly-His peptide chelator synthesis, the resin was washed several times with DMF, DCM, and dried in vacuum. The product was cleaved from the resin by using 1 ml of 1% of TFA (trifluoroacetate) in DCM+1% water for 30 minutes. The protecting groups were removed by using 95% of TFA with water and 2.5% of triisopropylsilane. The acid was evaporated in vacuum over KOH pellets. The final product was washed several times with diethyl-ether and dried. The dried poly-His peptide chelator was dissolved in water and lyophilized.

An amount, 1 mg, of poly-His peptide chelator was dissolved in 2 ml of water, and activated with 1 mg of EDC for 30 minutes on ice at 0° C. The mixture was added to 1 mg of VEGF (vascular endothelial growth factor), and continued for another 2 hours at room temperature. The poly-His peptide-VEGF (chelator-biological moiety) product was separated from the reaction mixture using size exclusion liquid chromatography.

The poly-His peptide-VEGF (combination) chemical entity specie is a sub-group (i) type of chelator (C)-chemical entity (X=Y=drug or biological moiety) chelate type of coordination compound configuration, and is characterized by the structure of general formula: (C)-(Y), wherein (C) is the poly-His peptide chelator, and (Y) is the VEGF (vascular endothelial growth factor) (biological moiety) chemical entity specie.

### EXAMPLE 11

### Combined Activating (Via Ionizing and Charging) a Metal Surface (M) and Electrochemical Binding (Via Chelation) a Chelator-Biological Moiety (Combination) Chemical Entity Specie (a Chelate Type of Coordination Compound, (C)-(Y)), to the Activated Metal Surface

In a combined metal surface activating and chelator binding (chelating) procedure, the metal surface of a nickel-titanium (Ni—Ti) alloy stent (whose metal surface blocking was removed as described in Example 1), absent of metal surface blocking, was activated by using an electrochemical oxidation type of metal surface activation procedure, following which an exemplary chelator-biological moiety (combination) chemical entity specie (a chelate type of coordination compound) was bound (via chelation) to the activated metal surface by using an electrochemical type of chelator binding (chelating) procedure.

The nickel-titanium (Ni—Ti) stent was immersed into an electrochemical cell which housed an aqueous electrolytic fluid having 2 mg of poly-His peptide-VEGF (chelator-biological moiety) (prepared as described in Example 10) at neutral conditions (pH of 6-7). The nickel-titanium (Ni—Ti) stent was conductively attached or connected to the anode electrode terminal (for oxidation), with a non-corrosive metallic element conductively attached or connected to the corresponding cathode electrode terminal, with the ratio of cathode surface area and anode surface area being about two to one. Current density of about 0.5 amps per square inch was maintained between the stent and the cathode during the electrolysis procedure, which was performed for 15 minutes at a temperature of 30° C. Following the electrolysis procedure, the free poly-His peptide-VEGF (chelator-biological moiety) that was not bound to the stent was fully washed in water, and dried.

The present invention, as illustratively described and exemplified hereinabove, has several beneficial and advantageous aspects, characteristics, and features, which are based on or/and a consequence of, the above illustratively described main aspects of novelty and inventiveness. Moreover, the present invention as illustratively described and exemplified hereinabove, widens the scope of the field of medical implant technology, in general, and especially in the sub-fields of drug coated stent and drug eluting stent technologies, relating to the need for finding and providing a sufficiently effective, consistent, robust, and safe, solution to restenosis, in general, and in-stent restenosis, in particular. More particularly, with respect to aspects focusing on the types and physicochemical properties, characteristics, and behaviors, of coatings coated onto medical implants, such as stents, as an important part of producing drug (or biological moiety) coated or drug (or biological moiety) eluting medical implant devices and systems. Especially, regarding possible alternatives or substitutions, such as ‘polymer-free’ based types of coatings, to currently known and applied ‘polymer’ based types of coatings.

It is appreciated that certain aspects and characteristics of the invention, which are, for clarity, described in the context of separate embodiments, may also be provided in combination in a single embodiment. Conversely, various aspects and characteristics of the invention, which are, for brevity, described in the context of a single embodiment, may also be provided separately or in any suitable sub-combination.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the spirit and broad scope of the appended claims.

### BIBLIOGRAPHY

Batchelor, W., B., et al., Prog. Cardiovasc. Dis., 1998, 41: 35-49.
Bendeck, M. P., et al., Circ. Res., 1994, 75: 539-545.
Bhatia, V., Bhatia, R., and Dhindsa, S., Drug-Eluting Intra-Coronary Stents: Have We Got The Magic Bullet?, J. Postgrad. Med., 2003, 49: 291-296. On-line at: http://www.jpgmonline.com/article.asp?issn=0022-3859;year=2003;volume=49;issue=3;spage=29;epage=296;aulast=Bhatia.
Brown, D. L., et al., Circulation, 1995, 15: 2125-2131.
Cardiac. Consult., Fall 2001, Vol. XI, No. 3, Brachytherapy for In-Stent Restenosis.
Clowes, A. W., et al., Circ. Res., 1990, 67: 61-67.
Coats, W. D., et al., Semin. Interv. Cardiol., 1997, 2: 167-176.
Feldman, L. J., et al., Circulation, 2001, 103: 3117-3122.
Frake, P., Kazanjian, M., Mark, N., and Yu, L., Drug Eluting Stents, Spring 2004 Semester, BI 108 Web Pages Project, Class Projects in the course on Organ Replacement (BI 108) by the Division of Biology and Medicine at Brown University, USA. Lysaght, M. (Course Instructor). On-line at: http://biomed.brown.edu/Courses/BI108/BI1108.html.
Galis, Z. S., et al., J. Clin. Invest., 1994, 94: 2493-2503.
Gershlick, A., et al., Inhibition of Restenosis With a Paclitaxel-Eluting, Polymer-Free Coronary Stent—The European evaLUation of pacliTaxel Eluting Stent (ELUTES) Trial, Circulation, 2004, 109: 487-493. On-line at: http://www.ncbi.nlm.nih.gov/entrez/query.fcgi?cmd=Retrieve&db=PubMed&List_uids=14 744971 &dopt=Abstract.
Hay, E. D., ed., Cell Biology of Extracellular Matrix, New York, Plenum, 1981.
Henney, A., et al., Proc. Nat'l. Acad. Sci. USA, 1991, 88: 8154-8158.
Jackson, C. L., et al., Am. J. Pathol., 1993, 143: 1024-1031.
Kridel, S. J., JBC, 2001, 276: 20572-20578.
McClean, D. R., Eigler, N. L., Rev. Cardiovasc. Med., 2002, 3(suppl 5): S16-S22.
McDonald, J. A., Cell Biol., 1988, 4: 183-208.
Mucha, A., et. al., JBC, 1998, 273: 2763.
Murphy, G., et. al., JBC, 1994, 269: 6632.
Netzel-Arnett, S., et al., JBC, 1991, 266: 6747-6755.
Netzel-Arnett, S., Anal. Biochem., 1991, 195: 86-92.
Netzel-Arnett, S., et. al., Biochemistry, 1993, 32: 6427-6432.
Niedzwiecki, L., et al., Biochemistry, 1992, 31: 12618-12623.
Piez, K. A., Reddi, A. H., eds., Extracellular Matrix Biochemistry, New York, Elsevier, 1984.
Porter, K. E., et al., Biochemical Society Transactions, 2002, 30: 120-126.
Reidy, M. A., et al., Circ. Res., 1996, 78: 405-414.
Ross, R., Atherosclerosis, 1997, 131: S3-S4.
Sawada, M., et al., Stroke, 1999, 30: 644-650.
Shofuda, K., et al., Lab. Invest., 1998, 78: 915-923.
Smouse, H. Bob, “Drug Eluting Stents”, MIIT, 2003. On-line at: http://www.miit.com/PDF/MIIT %202003/Drug %20eluting %20stents.pdf.
Whittaker, M., et al., Chem. Rev., 1999, 99: 2735-2776.
Zempo, N., et al., J. Vasc. Surg., 1994, 20: 209-217.
Zorina, S., et al., Circ. Res., 2002, 90: 251-262.

What is claimed is:

1. A medical device comprising a medical implant component having a metal surface (M) to which is bound a chemical entity (X) via a chelator (C) chelated to said metal surface in an (M)-(C)-(X) configuration.

2. The medical device of claim 1, wherein said medical implant component corresponds to at least a section of at least a part having said metal surface of a whole medical implant.

3. The medical device of claim 2, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

4. The medical device of claim 2, wherein said medical implant is a stent and said part is selected from the group consisting of a metal wire, a metal filament, a metal thread, of said stent; a metal film, a metal plating, and a metal coating, deposited upon at least a section of another part of said stent.

5. The medical device of claim 2, wherein said medical implant is a prosthesis and said part is selected from the group consisting of a metal plate, a metal joint, a metal fin, a metal screw, a metal spike, a metal wire, a metal filament, a metal thread, a metal anchor, another metallic bone fixation element, of said prosthesis; a metal film, a metal plating, and a metal coating, deposited upon at least a section of another part of said prosthesis.

6. The medical device of claim 1, wherein said metal surface corresponds to an external side or/and an internal side of said medical implant component.

7. The medical device of claim 1, wherein said metal surface (M) there is a sub-population of exposed surface metal ions and atoms each being charged, uncharged, or polarized, and each being chelated to at least one chelator molecule of said chelator (C) in a form of a said metal surface (M)—said chelator (C) chelate type of coordination compound configuration.

8. The medical device of claim 7, wherein a population of said metal chelated chelator molecules of said chelator (C), there is a sub-population of said metal chelated chelator molecules each being bonded to, or at least interacting in a bonding-like manner with, at least one chemical entity specie of said chemical entity (X) in a form of a said metal surface (M)—said chelator (C)—said chemical entity (X) chelate type of coordination compound configuration.

9. The medical device of claim 8, wherein a population of said chelator bonded or interacting chemical entity species of said chemical entity (X), there is a sub-population of said chelator bonded or interacting chemical entity species each being additionally bonded to, or at least interacting in a bonding-like manner with, at least one other chemical entity specie of said chemical entity (X) in said form of said metal surface (M)—said chelator (C)—said chemical entity (X) chelate type of coordination compound configuration.

10. The medical device of claim 1, wherein said medical implant component includes a chelate type of coordination compound characterized by having a structure of general formula (C)-(X), wherein said (C) is said chelator and said (X) is said chemical entity chelated to said chelator in a chelate type of coordination compound configuration.

11. The medical device of claim 1, wherein said metal surface (M) each chelated surface metal ion or atom is chelated to at least one chelator molecule of said chelator (C) in a form of a said metal surface (M)—said chelator (C) chelate type of coordination compound configuration.

12. The medical device of claim 1, wherein said (M)-(C)-(X) configuration each chelator molecule of said chelator (C) has a negative charge, a zero charge, or a positive charge.

13. The medical device of claim 1, wherein said (M)-(C)-(X) configuration each said metal surface (M)—said chelator (C) chelate type of coordination compound configuration formed between at least one surface metal ion or atom of said metal surface (M) and at least one chelator molecule of said chelator (C) has a total zero, positive, or negative, net charge.

14. The medical device of claim 1, wherein coordination number of each chelated surface metal ion or atom of said metal surface (M) is in a range of between two and twelve.

15. The medical device of claim 1, wherein said (M)-(C)-(X) configuration, bonding or at least bonding-like interaction between a metal chelated chelator molecule of said chelator (C) and a said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

16. The medical device of claim 1, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

17. The medical device of claim 1, wherein said (M)-(C)-(X) configuration, bonding or at least bonding-like interaction between a metal chelated chelator molecule of said chelator (C) and a chemical entity specie of said chemical entity (X) is selected from the group consisting of being stable, and being selectively cleavable via an appropriate bond cleaving mechanism and a corresponding bond cleaving agent.

18. The medical device of claim 17, wherein said bond cleavage results in separation, elution, and migration, of said chemical entity specie of said chemical entity (X) away from said metal chelated chelator molecule of said chelator (C).

19. The medical device of claim 1, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of being stable, and being selectively cleavable via an appropriate bond cleaving mechanism and a corresponding bond cleaving agent.

20. The medical device of claim 19, wherein said bond cleavage results in separation, elution, and migration, of said additional chemical entity specie away from said chemical entity specie of said chemical entity (X).

21. The medical device of claim 1, wherein mass and molar quantities of at least a sub-combination of a component of said chelator (C) or/and of said chemical entity (X) in said (M)-(C)-(X) configuration bound on said metal surface (M) in a form of a surface coating are greater than 100 picograms and greater than 1 picomole, respectively, per square centimeter of said metal surface (M).

22. The medical device of claim 1, wherein said metal surface (M) is composed of a material selected from the group consisting of a metallic material, a semi-metallic material, and a combination thereof.

23. The medical device of claim 22, wherein said material includes at least one metal element, at least one metal alloy each of at least two metal elements, or a combination thereof.

24. The medical device of claim 23, wherein said at least one metal element is selected from the group consisting of titanium [Ti], vanadium [V], chromium [Cr], iron [Fe], cobalt [Co], nickel [Ni], copper [Cu], zinc [Zn], niobium [Nb], molybdenum [Mo], rhodium [Rh], palladium [Pd], silver [Ag], tantalum [Ta], tungsten [W], rhenium [Re], osmium [Os], iridium [Ir], platinum [Pt], gold [Au], beryllium [Be], and aluminum [Al].

25. The medical device of claim 23, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

26. The medical device of claim 1, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

27. The medical device of claim 1, wherein a type of chemical entity specie of said chemical entity (X) is a drug or a biological moiety.

28. The medical device of claim 27, wherein said drug is used for preventing or/and treating a cardiovascular type of medical condition of a subject.

29. The medical device of claim 28, wherein said medical condition of said subject is selected from the group consisting of restenosis, in-stent restenosis, thrombosis, and a combination thereof.

30. The medical device of claim 27, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

31. The medical device of claim 27, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

32. The medical device of claim 27, wherein said biological moiety is selected from the group consisting of proteins, lipids (fats), sugars, nucleic acids, antibodies, cells, cellular structures, cellular components, and combinations thereof.

33. The medical device of claim 32, wherein said protein is selected from the group consisting of enzymes, growth factors, hormones, cytokines, and combinations thereof.

34. The medical device of claim 32, wherein said lipid (fat) is selected from the group consisting of phospholipids, glycolipids, steroids, and combinations thereof.

35. The medical device of claim 32, wherein said sugar is selected from the group consisting of heparin, chondritin, glycogen, and combinations thereof.

36. The medical device of claim 32, wherein said nucleic acid is selected from the group consisting of deoxoribonucleic acid (DNA), ribonucleic acid (RNA), peptide nucleic acid (PNA), and combinations thereof.

37. The medical device of claim 32, wherein said antibody is selected from the group consisting of polyclonal antibodies, monoclonal antibodies, Fab fragments, and combinations thereof.

38. The medical device of claim 1, wherein a type of chemical entity specie of said chemical entity (X) is a linker.

39. The medical device of claim 38, wherein said linker is selected from the group consisting of peptides, lipids, and sugars.

40. The medical device of claim 38, wherein said linker is a substrate to, and is cleavable by, at least one type of an enzyme whose activity is induced or expressed during onset of a cardiovascular type of medical condition of a subject.

41. The medical device of claim 39, wherein said peptide type of said linker is a substrate to, and is cleavable by, a matrix metalloproteinase protease type of enzyme whose activity is induced or expressed during onset of a cardiovascular type of medical condition of a subject.

42. The medical device of claim 39, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

43. The medical device of claim 39, wherein said peptide type of said linker is a substrate to, and is cleavable by, a type of peptidase selected from the group consisting of serine-type peptidases, threonine-type peptidases, aspartic-type peptidases, and cystein-type peptidases.

44. The medical device of claim 39, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

45. The medical device of claim 39, wherein said sugar type of said linker is a substrate to, and is cleaved by, a type of sugar degrading enzyme selected from the group consisting of heparinase and hyaloronidase.

46. The medical device of claim 39, wherein said sugar type of said linker is selected from the group consisting of polysaccharide glycosaminoglycans, chlondroitin sulfate, dermatan sulfate, heparan sulfate, heparin, and keratan sulfate.

47. The medical device of claim 38, wherein said linker is a biocompatible synthetic polymer that is a substrate to, and is cleavable by, at least one type of a chemical whose activity is induced or expressed during onset of a cardiovascular type of medical condition of a subject.

48. The medical device of claim 47, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

49. The medical device of claim 38, wherein said linker is a biocompatible synthetic bi-functional cross-linker that is a substrate to, and is cleavable by, at least one type of a chemical whose activity is induced or expressed during onset of a cardiovascular type of medical condition of a subject.

50. The medical device of claim 49, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

51. A medical device comprising a medical implant component having a surface to which is bound a chemical at a surface concentration of greater than 100 picograms per cm².

52. The medical device of claim 51, wherein said medical implant component corresponds to at least a section of at least a part having said surface of a whole medical implant.

53. The medical device of claim 51, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

54. The medical device of claim 51, wherein said medical implant is a stent and said part is selected from the group consisting of a wire, a filament, a thread, of said stent; a film, a plating, and a coating, deposited upon at least a section of another part of said stent.

55. The medical device of claim 51, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

56. The medical device of claim 51, wherein said surface corresponds to an external side or/and an internal side of said medical implant component.

57. The medical device of claim 51, wherein said surface is composed of a material selected from the group consisting of a metallic material, a semi-metallic material, and a combination thereof.

58. The medical device of claim 57, wherein said material includes at least one metal element, at least one metal alloy each of at least two metal elements, or a combination thereof.

59. The medical device of claim 58, wherein said at least one metal element is selected from the group consisting of titanium [Ti], vanadium [V], chromium [Cr], iron [Fe], cobalt [Co], nickel [Ni], copper [Cu], zinc [Zn], niobium [Nb], molybdenum [Mo], rhodium [Rh], palladium [Pd], silver [Ag], tantalum [Ta], tungsten [W], rhenium [Re], osmium [Os], iridium [Ir], platinum [Pt], gold [Au], beryllium [Be], and aluminum [Al].

60. The medical device of claim 58, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

61. The medical device of claim 51, wherein said chemical is selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid, 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

62. The medical device of claim 51, wherein a type of chemical entity specie of said chemical is a drug or a biological moiety.

63. The medical device of claim 62, wherein said drug is used for preventing or/and treating a cardiovascular type of medical condition of a subject.

64. The medical device of claim 63, wherein said medical condition of said subject is selected from the group consisting of restenosis, in-stent restenosis, thrombosis, and a combination thereof.

65. The medical device of claim 62, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

66. The medical device of claim 62, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

67. The medical device of claim 62, wherein said biological moiety is selected from the group consisting of proteins, lipids (fats), sugars, nucleic acids, antibodies, cells, cellular structures, cellular components, and combinations thereof.

68. The medical device of claim 67, wherein said protein is selected from the group consisting of enzymes, growth factors, hormones, cytokines, and combinations thereof.

69. The medical device of claim 67, wherein said lipid (fat) is selected from the group consisting of phospholipids, glycolipids, steroids, and combinations thereof.

70. The medical device of claim 67, wherein said sugar is selected from the group consisting of heparin, chondritin, glycogen, and combinations thereof.

71. The medical device of claim 67, wherein said nucleic acid is selected from the group consisting of deoxoribonucleic acid (DNA), ribonucleic acid (RNA), peptide nucleic acid (PNA), and combinations thereof.

72. The medical device of claim 67, wherein said antibody is selected from the group consisting of polyclonal antibodies, monoclonal antibodies, Fab fragments, and combinations thereof.

73. The medical device of claim 51, wherein a type of chemical entity specie of said chemical is a linker.

74. The medical device of claim 73, wherein said linker is selected from the group consisting of peptides, lipids, and sugars.

75. The medical device of claim 73, wherein said linker is a substrate to, and is cleavable by, at least one type of an enzyme whose activity is induced or expressed during onset of a cardiovascular type of medical condition of a subject.

76. The medical device of claim 74, wherein said peptide type of said linker is a substrate to, and is cleavable by, a matrix metalloproteinase protease type of enzyme whose activity is induced or expressed during onset of a cardiovascular type of medical condition of a subject.

77. The medical device of claim 74, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

78. The medical device of claim 74, wherein said peptide type of said linker is a substrate to, and is cleavable by, a type of peptidase selected from the group consisting of serine-type peptidases, threonine-type peptidases, aspartic-type peptidases, and cystein-type peptidases.

79. The medical device of claim 74, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

80. The medical device of claim 74, wherein said sugar type of said linker is a substrate to, and is cleaved by, a type of sugar degrading enzyme selected from the group consisting of heparinase and hyaloronidase.

81. The medical device of claim 74, wherein said sugar type of said linker is selected from the group consisting of polysaccharide glycosaminoglycans, chlondroitin sulfate, dermatan sulfate, heparan sulfate, heparin, and keratan sulfate.

82. The medical device of claim 73, wherein said linker is a biocompatible synthetic polymer that is a substrate to, and is cleavable by, at least one type of a chemical whose activity is induced or expressed during onset of a cardiovascular type of medical condition of a subject.

83. The medical device of claim 82, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

84. The medical device of claim 73, wherein said linker is a biocompatible synthetic bi-functional cross-linker that is a substrate to, and is cleavable by, at least one type of a chemical whose activity is induced or expressed during onset of a cardiovascular type of medical condition of a subject.

85. The medical device of claim 84, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

86. A method of manufacturing a medical device comprising binding to a metal surface (M) of a medical implant component a chemical entity (X) via a chelator (C) in an (M)-(C)-(X) configuration.

87. The method of claim 86, further comprising the step of removing metal surface blocking from exposed surface metal atoms of said metal surface (M).

88. The method of claim 87, wherein said removing is performed by exposing said metal surface (M) to a base in liquid phase, followed by washing said base treated metal surface (M) with water.

89. The method of claim 88, wherein said base is an inorganic base selected from the group consisting of ammonium hydroxide, sodium hydroxide, and potassium hydroxide.

90. The method of claim 88, wherein said base is an organic base selected from the group consisting of piperidine, pyridine, triethylamine, propylamine, diisopropilamine, and dimethylaminoperidine.

91. The method of claim 86, further comprising the step of activating via ionizing and charging said metal surface (M), for forming an activated ionized and charged metal surface (M) capable of being chelated to said chelator (C) and for binding said chelator (C).

92. The method of claim 91, wherein said activating is performed by using a metal surface activation procedure selected from the group consisting of a chemical type of metal surface activation procedure, and an electrochemical type of metal surface activation procedure.

93. The method of claim 92, wherein a said chemical type of metal surface activation procedure is based on chemical oxidation involving use of at least one chemical oxidant or oxidizing reagent.

94. The method of claim 93, wherein said at least one chemical oxidant or oxidizing reagent is selected from the group consisting of chromates, nitrates, nitrites, persulfates, permanganates, periodates, oxygen, hydrogen peroxide, and combinations thereof.

95. The method of claim 92, wherein a said chemical type of metal surface activation procedure is based on chemical reduction involving use of at least one chemical reducer or reducing reagent.

96. The method of claim 92, wherein a said electrochemical type of metal surface activation procedure is based on electrochemical oxidation of said metal surface (M) taking place in an electrochemical cell housing an electrolytic fluid including at least one chemical oxidant or oxidizing reagent.

97. The method of claim 96, wherein a said chemical oxidant or oxidizing reagent is selected from the group consisting of hydrochloric acid, hydrobromic acid, hydrofluoric acid, sulfuric acid, phosphoric acid, perchloric acid, trifluoroacetic acid, oxalic acid, citric acid, and a combination thereof.

98. The method of claim 92, wherein a said electrochemical type of metal surface activation procedure is based on electrochemical reduction of said metal surface (M) taking place in an electrochemical cell housing an electrolytic fluid including at least one chemical reducer or reducing reagent.

99. The method of claim 91, further comprising the step of binding via chelation of said chelator (C) to said activated ionized and charged metal surface (M), for forming said metal surface (M) to which is chelated said chelator (C) in an (M)-(C) chelate type of coordination compound configuration.

100. The method of claim 99, wherein said binding is performed by using a chelator binding procedure selected from the group consisting of a chemical type of chelator binding procedure, and an electrochemical type of chelator binding procedure.

101. The method of claim 100, wherein a said chemical type of chelator binding procedure includes exposing said activated metal surface (M) to a liquid phase form of a chelator compound of said chelator (C).

102. The method of claim 99, wherein the step of binding is performed together with the step of activating said metal surface (M).

103. The method of claim 102, wherein said activating and said binding are performed together by using an electrochemical oxidation type of procedure.

104. The method of claim 86, further comprising the step of reactively combining a first chemical entity specie of said chemical entity (X), with a second chemical entity specie of said chemical entity (X), for forming a third chemical entity specie of said chemical entity (X).

105. The method of claim 104, wherein said first type of said chemical entity specie is a drug or a biological moiety and said second type of said chemical entity specie is a linker, such that said formed third type of said chemical entity specie is a linker-drug or a linker-biological moiety combination chemical entity specie.

106. The method of claim 105, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

107. The method of claim 105, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

108. The method of claim 105, wherein said biological moiety is selected from the group consisting of proteins, lipids (fats), sugars, nucleic acids, antibodies, cells, cellular structures, cellular components, and combinations thereof.

109. The method of claim 108, wherein said protein is selected from the group consisting of enzymes, growth factors, hormones, cytokines, and combinations thereof.

110. The method of claim 108, wherein said lipid (fat) is selected from the group consisting of phospholipids, glycolipids, steroids, and combinations thereof.

111. The method of claim 108, wherein said sugar is selected from the group consisting of heparin, chondritin, glycogen, and combinations thereof.

112. The method of claim 108, wherein said nucleic acid is selected from the group consisting of deoxoribonucleic acid (DNA), ribonucleic acid (RNA), peptide nucleic acid (PNA), and combinations thereof.

113. The method of claim 108, wherein said antibody is selected from the group consisting of polyclonal antibodies, monoclonal antibodies, Fab fragments, and combinations thereof.

114. The method of claim 105, wherein said linker is selected from the group consisting of peptides, lipids, and sugars.

115. The method of claim 105, wherein said linker is a substrate to, and is cleavable by, at least one type of an enzyme whose activity is induced or expressed during onset of a cardiovascular type of medical condition of a subject.

116. The method of claim 114, wherein said peptide type of said linker is a substrate to, and is cleavable by, a matrix metalloproteinase protease type of enzyme whose activity is induced or expressed during onset of a cardiovascular type of medical condition of a subject.

117. The method of claim 114, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

118. The method of claim 114, wherein said peptide type of said linker is a substrate to, and is cleavable by, a type of peptidase selected from the group consisting of serine-type peptidases, threonine-type peptidases, aspartic-type peptidases, and cystein-type peptidases.

119. The method of claim 114, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

120. The method of claim 114, wherein said sugar type of said linker is a substrate to, and is cleaved by, a type of sugar degrading enzyme selected from the group consisting of heparinase and hyaloronidase.

121. The method of claim 114, wherein said sugar type of said linker is selected from the group consisting of polysaccharide glycosaminoglycans, chlondroitin sulfate, dermatan sulfate, heparan sulfate, heparin, and keratan sulfate.

122. The method of claim 105, wherein said linker is a biocompatible synthetic polymer that is a substrate to, and is cleavable by, at least one type of a chemical whose activity is induced or expressed during onset of a cardiovascular type of medical condition of a subject.

123. The method of claim 122, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

124. The method of claim 105, wherein said linker is a biocompatible synthetic bi-functional cross-linker that is a substrate to, and is cleavable by, at least one type of a chemical whose activity is induced or expressed during onset of a cardiovascular type of medical condition of a subject.

125. The method of claim 124, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

126. The method of claim 104, further comprising the step of binding said third chemical entity specie of said chemical entity (X) to said chelator (C) bound to said metal surface (M).

127. The method of claim 86, wherein said medical implant component corresponds to at least a section of at least a part having said metal surface of a whole medical implant.

128. The method of claim 127, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

129. The method of claim 127, wherein said medical implant is a stent and said part is selected from the group consisting of a wire, a filament, a thread, of said stent; a film, a plating, and a coating, deposited upon at least a section of another part of said stent.

130. The method of claim 127, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

131. The method of claim 86, wherein said metal surface corresponds to an external side or/and an internal side of said medical implant component.

132. The method of claim 86, wherein said metal surface (M) there is a sub-population of exposed surface metal ions and atoms each being charged, uncharged, or polarized, and each being chelated to at least one chelator molecule of said chelator (C) in a form of a said metal surface (M)—said chelator (C) chelate type of coordination compound configuration.

133. The method of claim 132, wherein a population of said metal chelated chelator molecules of said chelator (C), there is a sub-population of said metal chelated chelator molecules each being bonded to, or at least interacting in a bonding-like manner with, at least one chemical entity specie of said chemical entity (X) in a form of a said metal surface (M)—said chelator (C)—said chemical entity (X) chelate type of coordination compound configuration.

134. The method of claim 133, wherein a population of said chelator bonded or interacting chemical entity species of said chemical entity (X), there is a sub-population of said chelator bonded or interacting chemical entity species each being additionally bonded to, or at least interacting in a bonding-like manner with, at least one other chemical entity specie of said chemical entity (X) in said form of said metal surface (M)—said chelator (C)—said chemical entity (X) chelate type of coordination compound configuration.

135. The method of claim 86, wherein said medical implant component includes a chelate type of coordination compound characterized by having a structure of general formula (C)-(X), wherein said (C) is said chelator and said (X) is said chemical entity chelated to said chelator in a chelate type of coordination compound configuration.

136. The method of claim 86, wherein said metal surface (M) each chelated surface metal ion or atom is chelated to at least one chelator molecule of said chelator (C) in a form of a said metal surface (M)—said chelator (C) chelate type of coordination compound configuration.

137. The method of claim 86, wherein said (M)-(C)-(X) configuration each chelator molecule of said chelator (C) has a negative charge, a zero charge, or a positive charge.

138. The method of claim 86, wherein said (M)-(C)-(X) configuration each said metal surface (M)—said chelator (C) chelate type of coordination compound configuration formed between at least one surface metal ion or atom of said metal surface (M) and at least one chelator molecule of said chelator (C) has a total zero, positive, or negative, net charge.

139. The method of claim 86, wherein coordination number of each chelated surface metal ion or atom of said metal surface (M) is in a range of between two and twelve.

140. The method of claim 86, wherein said (M)-(C)-(X) configuration, bonding or at least bonding-like interaction between a metal chelated chelator molecule of said chelator (C) and a said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

141. The method of claim 86, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

142. The method of claim 86, wherein said (M)-(C)-(X) configuration, bonding or at least bonding-like interaction between a metal chelated chelator molecule of said chelator (C) and a chemical entity specie of said chemical entity (X) is selected from the group consisting of being stable, and being selectively cleavable via an appropriate bond cleaving mechanism and a corresponding bond cleaving agent.

143. The method of claim 142, wherein said bond cleavage results in separation, elution, and migration, of said chemical entity specie of said chemical entity (X) away from said metal chelated chelator molecule of said chelator (C).

144. The method of claim 86, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of being stable, and being selectively cleavable via an appropriate bond cleaving mechanism and a corresponding bond cleaving agent.

145. The method of claim 144, wherein said bond cleavage results in separation, elution, and migration, of said additional chemical entity specie away from said chemical entity specie of said chemical entity (X).

146. The method of claim 86, wherein mass and molar quantities of at least a sub-combination of a component of said chelator (C) or/and of said chemical entity (X) in said (M)-(C)-(X) configuration bound on said metal surface (M) in a form of a surface coating are greater than 100 picograms and greater than 1 picomole, respectively, per square centimeter of said metal surface (M).

147. The method of claim 86, wherein said metal surface (M) is composed of a material selected from the group consisting of a metallic material, a semi-metallic material, and a combination thereof.

148. The method of claim 147, wherein said material includes at least one metal element, at least one metal alloy each of at least two metal elements, or a combination thereof.

149. The method of claim 148, wherein said at least one metal element is selected from the group consisting of titanium [Ti], vanadium [V], chromium [Cr], iron [Fe], cobalt [Co], nickel [Ni], copper [Cu], zinc [Zn], niobium [Nb], molybdenum [Mo], rhodium [Rh], palladium [Pd], silver [Ag], tantalum [Ta], tungsten [W], rhenium [Re], osmium [Os], iridium [Ir], platinum [Pt], gold [Au], beryllium [Be], and aluminum [Al].

150. The method of claim 148, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

151. The method of claim 86, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

152. The method of claim 86, wherein a type of chemical entity specie of said chemical entity (X) is a drug or a biological moiety.

153. The method of claim 152, wherein said drug is used for preventing or/and treating a cardiovascular type of medical condition of a subject.

154. The method of claim 153, wherein said medical condition of said subject is selected from the group consisting of restenosis, in-stent restenosis, thrombosis, and a combination thereof.

155. The method of claim 152, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

156. The method of claim 152, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

157. The method of claim 152, wherein said biological moiety is selected from the group consisting of proteins, lipids (fats), sugars, nucleic acids, antibodies, cells, cellular structures, cellular components, and combinations thereof.

158. The method of claim 157, wherein said protein is selected from the group consisting of enzymes, growth factors, hormones, cytokines, and combinations thereof.

159. The method of claim 157, wherein said lipid (fat) is selected from the group consisting of phospholipids, glycolipids, steroids, and combinations thereof.

160. The method of claim 157, wherein said sugar is selected from the group consisting of heparin, chondritin, glycogen, and combinations thereof.

161. The method of claim 157, wherein said nucleic acid is selected from the group consisting of deoxoribonucleic acid (DNA), ribonucleic acid (RNA), peptide nucleic acid (PNA), and combinations thereof.

162. The method of claim 157, wherein said antibody is selected from the group consisting of polyclonal antibodies, monoclonal antibodies, Fab fragments, and combinations thereof.

163. The method of claim 86, wherein a type of chemical entity specie of said chemical entity (X) is a linker.

164. The method of claim 163, wherein said linker is selected from the group consisting of peptides, lipids, and sugars.

165. The method of claim 163, wherein said linker is a substrate to, and is cleavable by, at least one type of an enzyme whose activity is induced or expressed during onset of a cardiovascular type of medical condition of a subject.

166. The method of claim 164, wherein said peptide type of said linker is a substrate to, and is cleavable by, a matrix metalloproteinase protease type of enzyme whose activity is induced or expressed during onset of a cardiovascular type of medical condition of a subject.

167. The method of claim 164, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

168. The method of claim 164, wherein said peptide type of said linker is a substrate to, and is cleavable by, a type of peptidase selected from the group consisting of serine-type peptidases, threonine-type peptidases, aspartic-type peptidases, and cystein-type peptidases.

169. The method of claim 164, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

170. The method of claim 164, wherein said sugar type of said linker is a substrate to, and is cleaved by, a type of sugar degrading enzyme selected from the group consisting of heparinase and hyaloronidase.

171. The method of claim 164, wherein said sugar type of said linker is selected from the group consisting of polysaccharide glycosaminoglycans, chlondroitin sulfate, dermatan sulfate, heparan sulfate, heparin, and keratan sulfate.

172. The method of claim 163, wherein said linker is a biocompatible synthetic polymer that is a substrate to, and is cleavable by, at least one type of a chemical whose activity is induced or expressed during onset of a cardiovascular type of medical condition of a subject.

173. The method of claim 172, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

174. The method of claim 163, wherein said linker is a biocompatible synthetic bi-functional cross-linker that is a substrate to, and is cleavable by, at least one type of a chemical whose activity is induced or expressed during onset of a cardiovascular type of medical condition of a subject.

175. The method of claim 174, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

176. A medical implant system comprising:
(a) a medical implant component having a metal surface (M) to which is bound a chemical entity (X) via a chelator (C) chelated to said metal surface in an (M)-(C)-(X) configuration; and
(b) a delivery device for delivering said medical implant component to a pre-determined position in a subject.

177. The medical implant system of claim 176, wherein said medical implant component corresponds to at least a section of at least a part having said metal surface of a whole medical implant.

178. The medical implant system of claim 177, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

179. The medical implant system of claim 177, wherein said medical implant is a stent and said part is selected from the group consisting of a wire, a filament, a thread, of said stent; a film, a plating, and a coating, deposited upon at least a section of another part of said stent.

180. The medical implant system of claim 177, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

181. The medical implant system of claim 176, wherein said metal surface corresponds to an external side or/and an internal side of said medical implant component.

182. The medical implant system of claim 176, wherein said metal surface (M) there is a sub-population of exposed surface metal ions and atoms each being charged, uncharged, or polarized, and each being chelated to at least one chelator molecule of said chelator (C) in a form of a said metal surface (M)—said chelator (C) chelate type of coordination compound configuration.

183. The medical implant system of claim 182, wherein a population of said metal chelated chelator molecules of said chelator (C), there is a sub-population of said metal chelated chelator molecules each being bonded to, or at least interacting in a bonding-like manner with, at least one chemical entity specie of said chemical entity (X) in a form of a said metal surface (M)—said chelator (C)—said chemical entity (X) chelate type of coordination compound configuration.

184. The medical implant system of claim 183, wherein a population of said chelator bonded or interacting chemical entity species of said chemical entity (X), there is a sub-population of said chelator bonded or interacting chemical entity species each being additionally bonded to, or at least interacting in a bonding-like manner with, at least one other chemical entity specie of said chemical entity (X) in said form of said metal surface (M)—said chelator (C)—said chemical entity (X) chelate type of coordination compound configuration.

185. The medical implant system of claim 176, wherein said medical implant component includes a chelate type of coordination compound characterized by having a structure of general formula (C)—(X), wherein said (C) is said chelator and said (X) is said chemical entity chelated to said chelator in a chelate type of coordination compound configuration.

186. The medical implant system of claim 176, wherein said metal surface (M) each chelated surface metal ion or atom is chelated to at least one chelator molecule of said chelator (C) in a form of a said metal surface (M)—said chelator (C) chelate type of coordination compound configuration.

187. The medical implant system of claim 176, wherein said (M)-(C)-(X) configuration each chelator molecule of said chelator (C) has a negative charge, a zero charge, or a positive charge.

188. The medical implant system of claim 176, wherein said (M)-(C)-(X) configuration each said metal surface (M)—said chelator (C) chelate type of coordination compound configuration formed between at least one surface metal ion or atom of said metal surface (M) and at least one chelator molecule of said chelator (C) has a total zero, positive, or negative, net charge.

189. The medical implant system of claim 176, wherein coordination number of each chelated surface metal ion or atom of said metal surface (M) is in a range of between two and twelve.

190. The medical implant system of claim 176, wherein said (M)-(C)-(X) configuration, bonding or at least bonding-like interaction between a metal chelated chelator molecule of said chelator (C) and a said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

191. The medical implant system of claim 176, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

192. The medical implant system of claim 176, wherein said (M)-(C)-(X) configuration, bonding or at least bonding-like interaction between a metal chelated chelator molecule of said chelator (C) and a chemical entity specie of said chemical entity (X) is selected from the group consisting of being stable, and being selectively cleavable via an appropriate bond cleaving mechanism and a corresponding bond cleaving agent.

193. The medical implant system of claim 192, wherein said bond cleavage results in separation, elution, and migration, of said chemical entity specie of said chemical entity (X) away from said metal chelated chelator molecule of said chelator (C).

194. The medical implant system of claim 176, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of being stable, and being selectively cleavable via an appropriate bond cleaving mechanism and a corresponding bond cleaving agent.

195. The medical implant system of claim 194, wherein said bond cleavage results in separation, elution, and migration, of said additional chemical entity specie away from said chemical entity specie of said chemical entity (X).

196. The medical implant system of claim 176, wherein mass and molar quantities of at least a sub-combination of a component of said chelator (C) or/and of said chemical entity (X) in said (M)-(C)-(X) configuration bound on said metal surface (M) in a form of a surface coating are greater than 100 picograms and greater than 1 picomole, respectively, per square centimeter of said metal surface (M).

197. The medical implant system of claim 176, wherein said metal surface (M) is composed of a material selected from the group consisting of a metallic material, a semi-metallic material, and a combination thereof.

198. The medical implant system of claim 197, wherein said material includes at least one metal element, at least one metal alloy each of at least two metal elements, or a combination thereof.

199. The medical implant system of claim 198, wherein said at least one metal element is selected from the group consisting of titanium [Ti], vanadium [V], chromium [Cr], iron [Fe], cobalt [Co], nickel [Ni], copper [Cu], zinc [Zn], niobium [Nb], molybdenum [Mo], rhodium [Rh], palladium [Pd], silver [Ag], tantalum [Ta], tungsten [W], rhenium [Re], osmium [Os], iridium [Ir], platinum [Pt], gold [Au], beryllium [Be], and aluminum [Al].

200. The medical implant system of claim 198, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

201. The medical implant system of claim 176, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

202. The medical implant system of claim 176, wherein a type of chemical entity specie of said chemical entity (X) is a drug or a biological moiety.

203. The medical implant system of claim 202, wherein said drug is used for preventing or/and treating a cardiovascular type of medical condition of the subject.

204. The medical implant system of claim 203, wherein said medical condition of the subject is selected from the group consisting of restenosis, in-stent restenosis, thrombosis, and a combination thereof.

205. The medical implant system of claim 202, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

206. The medical implant system of claim 202, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

207. The medical implant system of claim 202, wherein said biological moiety is selected from the group consisting of proteins, lipids (fats), sugars, nucleic acids, antibodies, cells, cellular structures, cellular components, and combinations thereof.

208. The medical implant system of claim 207, wherein said protein is selected from the group consisting of enzymes, growth factors, hormones, cytokines, and combinations thereof.

209. The medical implant system of claim 207, wherein said lipid (fat) is selected from the group consisting of phospholipids, glycolipids, steroids, and combinations thereof.

210. The medical implant system of claim 207, wherein said sugar is selected from the group consisting of heparin, chondritin, glycogen, and combinations thereof.

211. The medical implant system of claim 207, wherein said nucleic acid is selected from the group consisting of deoxoribonucleic acid (DNA), ribonucleic acid (RNA), peptide nucleic acid (PNA), and combinations thereof.

212. The medical implant system of claim 207, wherein said antibody is selected from the group consisting of polyclonal antibodies, monoclonal antibodies, Fab fragments, and combinations thereof.

213. The medical implant system of claim 176, wherein a type of chemical entity specie of said chemical entity (X) is a linker.

214. The medical implant system of claim 213, wherein said linker is selected from the group consisting of peptides, lipids, and sugars.

215. The medical implant system of claim 213, wherein said linker is a substrate to, and is cleavable by, at least one type of an enzyme whose activity is induced or expressed during onset of a cardiovascular type of medical condition of the subject.

216. The medical implant system of claim 214, wherein said peptide type of said linker is a substrate to, and is cleavable by, a matrix metalloproteinase protease type of enzyme whose activity is induced or expressed during onset of a cardiovascular type of medical condition of the subject.

217. The medical implant system of claim 214, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

218. The medical implant system of claim 214, wherein said peptide type of said linker is a substrate to, and is cleavable by, a type of peptidase selected from the group consisting of serine-type peptidases, threonine-type peptidases, aspartic-type peptidases, and cystein-type peptidases.

219. The medical implant system of claim 214, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

220. The medical implant system of claim 214, wherein said sugar type of said linker is a substrate to, and is cleaved by, a type of sugar degrading enzyme selected from the group consisting of heparinase and hyaloronidase.

221. The medical implant system of claim 214, wherein said sugar type of said linker is selected from the group consisting of polysaccharide glycosaminoglycans, chlondroitin sulfate, dermatan sulfate, heparan sulfate, heparin, and keratan sulfate.

222. The medical implant system of claim 213, wherein said linker is a biocompatible synthetic polymer that is a substrate to, and is cleavable by, at least one type of a chemical whose activity is induced or expressed during onset of a cardiovascular type of medical condition of the subject.

223. The medical implant system of claim 222, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

224. The medical implant system of claim 213, wherein said linker is a biocompatible synthetic bi-functional cross-linker that is a substrate to, and is cleavable by, at least one type of a chemical whose activity is induced or expressed during onset of a cardiovascular type of medical condition of the subject.

225. The medical implant system of claim 224, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

226. The medical implant system of claim 176, wherein said delivery device is selected from the group consisting of a stent type of delivery device, and a prosthesis type of delivery device.

227. The medical implant system of claim 176, wherein said delivery device is selected from the group consisting of a drug coated stent type of delivery device, and a drug eluting stent type of delivery device.

228. The medical implant system of claim 176, wherein said delivery device is in a form of a balloon catheter.

229. The medical implant system of claim 176, wherein said pre-determined position in the subject is at a location inside a cavity of a blood vessel of the subject.

230. The medical implant system of claim 176, wherein said pre-determined position in the subject is at a location inside a socket or connection of a limb, bone, or other body part of the subject.

231. A method of implanting a medical device comprising, implanting in a subject in need thereof a medical device which comprises a medical implant component having a metal surface (M) to which is bound a chemical entity (X) via a chelator (C) chelated to said metal surface in an (M)-(C)-(X) configuration.

232. The method of claim 231, further comprising delivering said medical implant component to a pre-determined position in the subject.

233. The method of claim 232, wherein said pre-determined position in the subject is at a location inside a cavity of a blood vessel of the subject.

234. The method of claim 232, wherein said pre-determined position in the subject is at a location inside a socket or connection of a limb, bone, or other body part of the subject.

235. The method of claim 232, wherein a delivery device is used for said delivering.

236. The method of claim 235, wherein said delivery device is selected from the group consisting of a stent type of delivery device, and a prosthesis type of delivery device.

237. The method of claim 235, wherein said delivery device is selected from the group consisting of a drug coated stent type of delivery device, and a drug eluting stent type of delivery device.

238. The method of claim 235, wherein said delivery device is in a form of a balloon catheter.

239. The method of claim 231, wherein said medical implant component corresponds to at least a section of at least a part having said metal surface of a whole medical implant.

240. The method of claim 239, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

241. The method of claim 239, wherein said medical implant is a stent and said part is selected from the group consisting of a wire, a filament, a thread, of said stent; a film, a plating, and a coating, deposited upon at least a section of another part of said stent.

242. The method of claim 239, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

243. The method of claim 231, wherein said metal surface corresponds to an external side or/and an internal side of said medical implant component.

244. The method of claim 231, wherein said metal surface (M) there is a sub-population of exposed surface metal ions and atoms each being charged, uncharged, or polarized, and each being chelated to at least one chelator molecule of said chelator (C) in a form of a said metal surface (M)—said chelator (C) chelate type of coordination compound configuration.

245. The method of claim 244, wherein a population of said metal chelated chelator molecules of said chelator (C), there is a sub-population of said metal chelated chelator molecules each being bonded to, or at least interacting in a bonding-like manner with, at least one chemical entity specie of said chemical entity (X) in a form of a said metal surface (M)—said chelator (C)—said chemical entity (X) chelate type of coordination compound configuration.

246. The method of claim 245, wherein a population of said chelator bonded or interacting chemical entity species of said chemical entity (X), there is a sub-population of said chelator bonded or interacting chemical entity species each being additionally bonded to, or at least interacting in a bonding-like manner with, at least one other chemical entity specie of said chemical entity (X) in said form of said metal surface (M)—said chelator (C)—said chemical entity (X) chelate type of coordination compound configuration.

247. The method of claim 231, wherein said medical implant component includes a chelate type of coordination compound characterized by having a structure of general formula (C)-(X), wherein said (C) is said chelator and said (X) is said chemical entity chelated to said chelator in a chelate type of coordination compound configuration.

248. The method of claim 231, wherein said metal surface (M) each chelated surface metal ion or atom is chelated to at least one chelator molecule of said chelator (C) in a form of a said metal surface (M)—said chelator (C) chelate type of coordination compound configuration.

249. The method of claim 231, wherein said (M)-(C)-(X) configuration each chelator molecule of said chelator (C) has a negative charge, a zero charge, or a positive charge.

250. The method of claim 231, wherein said (M)-(C)-(X) configuration each said metal surface (M)—said chelator (C) chelate type of coordination compound configuration formed between at least one surface metal ion or atom of said metal surface (M) and at least one chelator molecule of said chelator (C) has a total zero, positive, or negative, net charge.

251. The method of claim 231, wherein coordination number of each chelated surface metal ion or atom of said metal surface (M) is in a range of between two and twelve.

252. The method of claim 231, wherein said (M)-(C)-(X) configuration, bonding or at least bonding-like interaction between a metal chelated chelator molecule of said chelator (C) and a said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

253. The method of claim 231, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

254. The method of claim 231, wherein said (M)-(C)-(X) configuration, bonding or at least bonding-like interaction between a metal chelated chelator molecule of said chelator (C) and a chemical entity specie of said chemical entity (X) is selected from the group consisting of being stable, and being selectively cleavable via an appropriate bond cleaving mechanism and a corresponding bond cleaving agent.

255. The method of claim 254, wherein said bond cleavage results in separation, elution, and migration, of said chemical entity specie of said chemical entity (X) away from said metal chelated chelator molecule of said chelator (C).

256. The method of claim 231, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of being stable, and being selectively cleavable via an appropriate bond cleaving mechanism and a corresponding bond cleaving agent.

257. The method of claim 256, wherein said bond cleavage results in separation, elution, and migration, of said additional chemical entity specie away from said chemical entity specie of said chemical entity (X).

258. The method of claim 231, wherein mass and molar quantities of at least a sub-combination of a component of said chelator (C) or/and of said chemical entity (X) in said (M)-(C)-(X) configuration bound on said metal surface (M) in a form of a surface coating are greater than 100 picograms and greater than 1 picomole, respectively, per square centimeter of said metal surface (M).

259. The method of claim 231, wherein said metal surface (M) is composed of a material selected from the group consisting of a metallic material, a semi-metallic material, and a combination thereof.

260. The method of claim 259, wherein said material includes at least one metal element, at least one metal alloy each of at least two metal elements, or a combination thereof.

261. The method of claim 260, wherein said at least one metal element is selected from the group consisting of titanium [Ti], vanadium [V], chromium [Cr], iron [Fe], cobalt [Co], nickel [Ni], copper [Cu], zinc [Zn], niobium [Nb], molybdenum [Mo], rhodium [Rh], palladium [Pd], silver [Ag], tantalum [Ta], tungsten [W], rhenium [Re], osmium [Os], iridium [Ir], platinum [Pt], gold [Au], beryllium [Be], and aluminum [Al].

262. The method of claim 260, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

263. The method of claim 231, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

264. The method of claim 231, wherein a type of chemical entity specie of said chemical entity (X) is a drug or a biological moiety.

265. The method of claim 254, wherein said drug is used for preventing or/and treating a cardiovascular type of medical condition of the subject.

266. The method of claim 265, wherein said medical condition of the subject is selected from the group consisting of restenosis, in-stent restenosis, thrombosis, and a combination thereof.

267. The method of claim 264, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

268. The method of claim 264, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

269. The method of claim 264, wherein said biological moiety is selected from the group consisting of proteins, lipids (fats), sugars, nucleic acids, antibodies, cells, cellular structures, cellular components, and combinations thereof.

270. The method of claim 269, wherein said protein is selected from the group consisting of enzymes, growth factors, hormones, cytokines, and combinations thereof.

271. The method of claim 269, wherein said lipid (fat) is selected from the group consisting of phospholipids, glycolipids, steroids, and combinations thereof.

272. The method of claim 269, wherein said sugar is selected from the group consisting of heparin, chondritin, glycogen, and combinations thereof.

273. The method of claim 269, wherein said nucleic acid is selected from the group consisting of deoxoribonucleic acid (DNA), ribonucleic acid (RNA), peptide nucleic acid (PNA), and combinations thereof.

274. The method of claim 269, wherein said antibody is selected from the group consisting of polyclonal antibodies, monoclonal antibodies, Fab fragments, and combinations thereof.

275. The method of claim 231, wherein a type of chemical entity specie of said chemical entity (X) is a linker.

276. The method of claim 275, wherein said linker is selected from the group consisting of peptides, lipids, and sugars.

277. The method of claim 275, wherein said linker is a substrate to, and is cleavable by, at least one type of an enzyme whose activity is induced or expressed during onset of a cardiovascular type of medical condition of the subject.

278. The method of claim 276, wherein said peptide type of said linker is a substrate to, and is cleavable by, a matrix metalloproteinase protease type of enzyme whose activity is induced or expressed during onset of a cardiovascular type of medical condition of the subject.

279. The method of claim 276, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

280. The method of claim 276, wherein said peptide type of said linker is a substrate to, and is cleavable by, a type of peptidase selected from the group consisting of serine-type peptidases, threonine-type peptidases, aspartic-type peptidases, and cystein-type peptidases.

281. The method of claim 276, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

282. The method of claim 276, wherein said sugar type of said linker is a substrate to, and is cleaved by, a type of sugar degrading enzyme selected from the group consisting of heparinase and hyaloronidase.

283. The method of claim 276, wherein said sugar type of said linker is selected from the group consisting of polysaccharide glycosaminoglycans, chlondroitin sulfate, dermatan sulfate, heparan sulfate, heparin, and keratan sulfate.

284. The method of claim 275, wherein said linker is a biocompatible synthetic polymer that is a substrate to, and is cleavable by, at least one type of a chemical whose activity is induced or expressed during onset of a cardiovascular type of medical condition of the subject.

285. The method of claim 284, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

286. The method of claim 275, wherein said linker is a biocompatible synthetic bi-functional cross-linker that is a substrate to, and is cleavable by, at least one type of a chemical whose activity is induced or expressed during onset of a cardiovascular type of medical condition of the subject.

287. The method of claim 286, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

288. A method of implanting a medical device comprising, implanting in a subject in need thereof a medical device which comprises a medical implant component having a surface to which is bound a chemical at a surface concentration of greater than 100 picograms per cm².

289. The method of claim 288, further comprising delivering said medical implant component to a pre-determined position in the subject.

290. The method of claim 289, wherein said pre-determined position in the subject is at a location inside a cavity of a blood vessel of the subject.

291. The method of claim 289, wherein said pre-determined position in the subject is at a location inside a socket or connection of a limb, bone, or other body part of the subject.

292. The method of claim 289, wherein a delivery device is used for said delivering.

293. The method of claim 292, wherein said delivery device is selected from the group consisting of a stent type of delivery device, and a prosthesis type of delivery device.

294. The method of claim 292, wherein said delivery device is selected from the group consisting of a drug coated stent type of delivery device, and a drug eluting stent type of delivery device.

295. The method of claim 292, wherein said delivery device is in a form of a balloon catheter.

296. The method of claim 288, wherein said medical implant component corresponds to at least a section of at least a part having said metal surface of a whole medical implant.

297. The method of claim 296, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

298. The method of claim 296, wherein said medical implant is a stent and said part is selected from the group consisting of a wire, a filament, a thread, of said stent; a film, a plating, and a coating, deposited upon at least a section of another part of said stent.

299. The method of claim 296, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

300. The method of claim 288, wherein said metal surface corresponds to an external side or/and an internal side of said medical implant component.

301. The method of claim 288, wherein said surface is composed of a material selected from the group consisting of a metallic material, a semi-metallic material, and a combination thereof.

302. The method of claim 301, wherein said material includes at least one metal element, at least one metal alloy each of at least two metal elements, or a combination thereof.

303. The method of claim 302, wherein said at least one metal element is selected from the group consisting of nickel [Ni], titanium [Ti], platinum [Pt], iridium [Ir], tantalum [Ta], iron [Fe], cobalt [Co], molybdenum [Mo], chromium [Cr], beryllium [Be], copper [Cu], tungsten [W], vanadium [V], niobium [Nb], palladium [Pd], gold [Au], silver [Ag], zinc [Zn], aluminum [Al], iron [Fe], and a combination thereof.

304. The method of claim 302, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

305. The method of claim 288, wherein said chemical is selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxi (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

306. The method of claim 288, wherein a type of chemical entity specie of said chemical is a drug or a biological moiety.

307. The method of claim 306, wherein said drug is used for preventing or/and treating a cardiovascular type of medical condition of the subject.

308. The method of claim 307, wherein said medical condition of the subject is selected from the group consisting of restenosis, in-stent restenosis, thrombosis, and a combination thereof.

309. The method of claim 306, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

310. The method of claim 306, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

311. The method of claim 306, wherein said biological moiety is selected from the group consisting of proteins, lipids (fats), sugars, nucleic acids, antibodies, cells, cellular structures, cellular components, and combinations thereof.

312. The method of claim 311, wherein said protein is selected from the group consisting of enzymes, growth factors, hormones, cytokines, and combinations thereof.

313. The method of claim 311, wherein said lipid (fat) is selected from the group consisting of phospholipids, glycolipids, steroids, and combinations thereof.

314. The method of claim 311, wherein said sugar is selected from the group consisting of heparin, chondritin, glycogen, and combinations thereof.

315. The method of claim 311, wherein said nucleic acid is selected from the group consisting of deoxoribonucleic acid (DNA), ribonucleic acid (RNA), peptide nucleic acid (PNA), and combinations thereof.

316. The method of claim 311, wherein said antibody is selected from the group consisting of polyclonal antibodies, monoclonal antibodies, Fab fragments, and combinations thereof.

317. The method of claim 288, wherein a type of chemical entity specie of said chemical is a linker.

318. The method of claim 317, wherein said linker is selected from the group consisting of peptides, lipids, and sugars.

319. The method of claim 317, wherein said linker is a substrate to, and is cleavable by, at least one type of an enzyme whose activity is induced or expressed during onset of a cardiovascular type of medical condition of the subject.

320. The method of claim 318, wherein said peptide type of said linker is a substrate to, and is cleavable by, a matrix metalloproteinase protease type of enzyme whose activity is induced or expressed during onset of a cardiovascular type of medical condition of the subject.

321. The method of claim 318, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

322. The method of claim 318, wherein said peptide type of said linker is a substrate to, and is cleavable by, a type of peptidase selected from the group consisting of serine-type peptidases, threonine-type peptidases, aspartic-type peptidases, and cystein-type peptidases.

323. The method of claim 318, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

324. The method of claim 318, wherein said sugar type of said linker is a substrate to, and is cleaved by, a type of sugar degrading enzyme selected from the group consisting of heparinase and hyaloronidase.

325. The method of claim 318, wherein said sugar type of said linker is selected from the group consisting of polysaccharide glycosaminoglycans, chlondroitin sulfate, dermatan sulfate, heparan sulfate, heparin, and keratan sulfate.

326. The method of claim 317, wherein said linker is a biocompatible synthetic polymer that is a substrate to, and is cleavable by, at least one type of a chemical whose activity is induced or expressed during onset of a cardiovascular type of medical condition of the subject.

327. The method of claim 326, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

328. The method of claim 317, wherein said linker is a biocompatible synthetic bi-functional cross-linker that is a substrate to, and is cleavable by, at least one type of a chemical whose activity is induced or expressed during onset of a cardiovascular type of medical condition of the subject.

329. The method of claim 318, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

330. A method of preventing or/and treating a medical condition of a subject, comprising implanting in the subject a medical device which comprises a medical implant component having a metal surface (M) to which is bound a chemical entity (X) via a chelator (C) chelated to said metal surface in an (M)-(C)-(X) configuration, such that activity of said bound chemical entity exhibits an efficacy for preventing or/and treating the medical condition.

331. The method of claim 330, further comprising delivering said medical implant component to a pre-determined position in the subject.

332. The method of claim 331, wherein said pre-determined position in the subject is at a location inside a cavity of a blood vessel of the subject.

333. The method of claim 331, wherein said pre-determined position in the subject is at a location inside a socket or connection of a limb, bone, or other body part of the subject.

334. The method of claim 331, wherein a delivery device is used for said delivering.

335. The method of claim 334, wherein said delivery device is selected from the group consisting of a stent type of delivery device, and a prosthesis type of delivery device.

336. The method of claim 334, wherein said delivery device is selected from the group consisting of a drug coated stent type of delivery device, and a drug eluting stent type of delivery device.

337. The method of claim 334, wherein said delivery device is in a form of a balloon catheter.

338. The method of claim 330, wherein said medical implant component corresponds to at least a section of at least a part having said metal surface of a whole medical implant.

339. The method of claim 338, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

340. The method of claim 338, wherein said medical implant is a stent and said part is selected from the group consisting of a wire, a filament, a thread, of said stent; a film, a plating, and a coating, deposited upon at least a section of another part of said stent.

341. The method of claim 338, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

342. The method of claim 330, wherein said metal surface corresponds to an external side or/and an internal side of said medical implant component.

343. The method of claim 330, wherein said metal surface (M) there is a sub-population of exposed surface metal ions and atoms each being charged, uncharged, or polarized, and each being chelated to at least one chelator molecule of said chelator (C) in a form of a said metal surface (M)—said chelator (C) chelate type of coordination compound configuration.

344. The method of claim 343, wherein a population of said metal chelated chelator molecules of said chelator (C), there is a sub-population of said metal chelated chelator molecules each being bonded to, or at least interacting in a bonding-like manner with, at least one chemical entity specie of said chemical entity (X) in a form of a said metal surface (M)—said chelator (C)—said chemical entity (X) chelate type of coordination compound configuration.

345. The method of claim 344, wherein a population of said chelator bonded or interacting chemical entity species of said chemical entity (X), there is a sub-population of said chelator bonded or interacting chemical entity species each being additionally bonded to, or at least interacting in a bonding-like manner with, at least one other chemical entity specie of said chemical entity (X) in said form of said metal surface (M)—said chelator (C)—said chemical entity (X) chelate type of coordination compound configuration.

346. The method of claim 330, wherein said medical implant component includes a chelate type of coordination compound characterized by having a structure of general formula (C)-(X), wherein said (C) is said chelator and said (X) is said chemical entity chelated to said chelator in a chelate type of coordination compound configuration.

347. The method of claim 330, wherein said metal surface (M) each chelated surface metal ion or atom is chelated to at least one chelator molecule of said chelator (C) in a form of a said metal surface (M)—said chelator (C) chelate type of coordination compound configuration.

348. The method of claim 330, wherein said (M)-(C)-(X) configuration each chelator molecule of said chelator (C) has a negative charge, a zero charge, or a positive charge.

349. The method of claim 330, wherein said (M)-(C)-(X) configuration each said metal surface (M)—said chelator (C) chelate type of coordination compound configuration formed between at least one surface metal ion or atom of said metal surface (M) and at least one chelator molecule of said chelator (C) has a total zero, positive, or negative, net charge.

350. The method of claim 330, wherein coordination number of each chelated surface metal ion or atom of said metal surface (M) is in a range of between two and twelve.

351. The method of claim 330, wherein said (M)-(C)-(X) configuration, bonding or at least bonding-like interaction between a metal chelated chelator molecule of said chelator (C) and a said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

352. The method of claim 330, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of at least one covalent bond, at least one ionic bond, at least one hydrogen bond, at least one van der Waals bond, at least one coordinate covalent bond, and a combination thereof.

353. The method of claim 330, wherein said (M)-(C)-(X) configuration, bonding or at least bonding-like interaction between a metal chelated chelator molecule of said chelator (C) and a chemical entity specie of said chemical entity (X) is selected from the group consisting of being stable, and being selectively cleavable via an appropriate bond cleaving mechanism and a corresponding bond cleaving agent.

354. The method of claim 353, wherein said bond cleavage results in separation, elution, and migration, of said chemical entity specie of said chemical entity (X) away from said metal chelated chelator molecule of said chelator (C).

355. The method of claim 330, wherein said (M)-(C)-(X) configuration, bonding or at least bonding-like interaction between a chelator bonded or interacting chemical entity specie of said chemical entity (X) and an additional said chemical entity specie of said chemical entity (X) is selected from the group consisting of being stable, and being selectively cleavable via an appropriate bond cleaving mechanism and a corresponding bond cleaving agent.

356. The method of claim 355, wherein said bond cleavage results in separation, elution, and migration, of said additional chemical entity specie away from said chemical entity specie of said chemical entity (X).

357. The method of claim 330, wherein mass and molar quantities of at least a sub-combination of a component of said chelator (C) or/and of said chemical entity (X) in said (M)-(C)-(X) configuration bound on said metal surface (M) in a form of a surface coating are greater than 100 picograms and greater than 1 picomole, respectively, per square centimeter of said metal surface (M).

358. The method of claim 330, wherein said metal surface (M) is composed of a material selected from the group consisting of a metallic material, a semi-metallic material, and a combination thereof.

359. The method of claim 358, wherein said material includes at least one metal element, at least one metal alloy each of at least two metal elements, or a combination thereof.

360. The method of claim 359, wherein said at least one metal element is selected from the group consisting of nickel [Ni], titanium [Ti], platinum [Pt], iridium [Ir], tantalum [Ta], iron [Fe], cobalt [Co], molybdenum [Mo], chromium [Cr], beryllium [Be], copper [Cu], tungsten [W], vanadium [V], niobium [Nb], palladium [Pd], gold [Au], silver [Ag], zinc [Zn], aluminum [Al], iron [Fe], and a combination thereof.

361. The method of claim 359, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

362. The method of claim 330, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

363. The method of claim 330, wherein a type of chemical entity specie of said chemical entity (X) is a drug or a biological moiety.

364. The method of claim 363, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

365. The method of claim 363, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

366. The method of claim 363, wherein said biological moiety is selected from the group consisting of proteins, lipids (fats), sugars, nucleic acids, antibodies, cells, cellular structures, cellular components, and combinations thereof.

367. The method of claim 366, wherein said protein is selected from the group consisting of enzymes, growth factors, hormones, cytokines, and combinations thereof.

368. The method of claim 366, wherein said lipid (fat) is selected from the group consisting of phospholipids, glycolipids, steroids, and combinations thereof.

369. The method of claim 366, wherein said sugar is selected from the group consisting of heparin, chondritin, glycogen, and combinations thereof.

370. The method of claim 366, wherein said nucleic acid is selected from the group consisting of deoxoribonucleic acid (DNA), ribonucleic acid (RNA), peptide nucleic acid (PNA), and combinations thereof.

371. The method of claim 366, wherein said antibody is selected from the group consisting of polyclonal antibodies, monoclonal antibodies, Fab fragments, and combinations thereof.

372. The method of claim 330, wherein a type of chemical entity specie of said chemical entity (X) is a linker.

373. The method of claim 372, wherein said linker is selected from the group consisting of peptides, lipids, and sugars.

374. The method of claim 372, wherein said linker is a substrate to, and is cleavable by, at least one type of an enzyme whose activity is induced or expressed during onset of a cardiovascular type of medical condition of the subject.

375. The method of claim 373, wherein said peptide type of said linker is a substrate to, and is cleavable by, a matrix metalloproteinase protease type of enzyme whose activity is induced or expressed during onset of a cardiovascular type of medical condition of the subject.

376. The method of claim 373, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

377. The method of claim 373, wherein said peptide type of said linker is a substrate to, and is cleavable by, a type of peptidase selected from the group consisting of serine-type peptidases, threonine-type peptidases, aspartic-type peptidases, and cystein-type peptidases.

378. The method of claim 373, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

379. The method of claim 373, wherein said sugar type of said linker is a substrate to, and is cleaved by, a type of sugar degrading enzyme selected from the group consisting of heparinase and hyaloronidase.

380. The method of claim 373, wherein said sugar type of said linker is selected from the group consisting of polysaccharide glycosaminoglycans, chlondroitin sulfate, dermatan sulfate, heparan sulfate, heparin, and keratan sulfate.

381. The method of claim 372, wherein said linker is a biocompatible synthetic polymer that is a substrate to, and is cleavable by, at least one type of a chemical whose activity is induced or expressed during onset of a cardiovascular type of medical condition of the subject.

382. The method of claim 381, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

383. The method of claim 382, wherein said linker is a biocompatible synthetic bi-functional cross-linker that is a substrate to, and is cleavable by, at least one type of a chemical whose activity is induced or expressed during onset of a cardiovascular type of medical condition of the subject.

384. The method of claim 383, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

385. The method of claim 330, wherein the medical condition of the subject is a cardiovascular type of medical condition.

386. The method of claim 330, wherein the medical condition of the subject is selected from the group consisting of restenosis, in-stent restenosis, thrombosis, and a combination thereof.

387. A chelate type of coordination compound comprising a structure of general formula:
(C)-(Y),
wherein (C) is a chelator and (Y) is a chemical entity selected from the group consisting of (i) a drug chelated to said chelator or a biological moiety chelated to said chelator, and, (ii) a linker having a first part chelated to said chelator and having a second part bonded to a drug or a biological moiety.

388. The coordination compound of claim 387, wherein said chemical entity (Y) is said drug or said biological moiety, such that configuration of said (C)-(Y) is characterized by having at least two coordinate covalent bonds between at least two coordinating groups of a chelator molecule of said chelator (C) and a chelated drug molecule or a biological moiety molecule of said chemical entity (Y).

389. The coordination compound of claim 387, wherein said chemical entity (Y) is said linker, such that configuration of said (C)-(Y) is characterized by having at least two coordinate covalent bonds between at least two coordinating groups of a chelator molecule of said chelator (C) and a first part of a chelated linker molecule of said chemical entity (Y).

390. The coordination compound of claim 389, wherein said configuration of said (C)-(Y) is further characterized by having at least one bond between second part of said chelated linker molecule and a drug molecule or a biological moiety molecule of said chemical entity (Y).

391. The coordination compound of claim 390, wherein bonding between said second part of said chelated linker molecule and said drug molecule or said biological moiety molecule of said chemical entity (Y) is selected from the group consisting of at least one covalent bond, at least one ionic bond, at least one hydrogen bond, at least one van der Waals bond, at least one coordinate covalent bond, and a combination thereof.

392. The coordination compound of claim 391, wherein bonding or at least bonding-like interaction between said second part of said chelated linker molecule and said drug molecule or said biological moiety molecule of said chemical entity (Y) is selected from the group consisting of being stable, and being selectively cleavable via an appropriate bond cleaving mechanism and a corresponding bond cleaving agent.

393. The coordination compound of claim 392, wherein said bond cleavage results in separation, elution, and migration, of said drug molecule or of said biological moiety molecule away from said second part of said chelated linker molecule.

394. The coordination compound of claim 387, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

395. The coordination compound of claim 387, wherein said drug is used for preventing or/and treating a cardiovascular type of medical condition of a subject.

396. The coordination compound of claim 395, wherein said medical condition of said subject is selected from the group consisting of restenosis, in-stent restenosis, thrombosis, and a combination thereof.

397. The coordination compound of claim 387, wherein said drug is selected from the group consisting of alpha-adrenergic blocking drugs, angiotensin converting enzyme inhibitor drugs, antiarrhythmic drugs, anticoagulant and antiplatelet drugs, antithrombotic or thrombin inhibitor drugs, beta-adrenergic blocking drugs, calcium channel blocking drugs, centrally acting drugs, cholesterol lowering agent drugs, digitalis drugs, diuretic drugs, nitrate drugs, peripheral adrenergic antagonist drugs, vasodilator drugs, and combination drugs thereof.

398. The coordination compound of claim 387, wherein said drug is selected from the group consisting of anti-neoplastic or anti-inflammatory drugs, immunosupressive or anti-proliferative drugs, migration inhibitor or ECM modulator drugs, and enhanced healing or re-endothelialization drugs.

399. The coordination compound of claim 387, wherein said biological moiety is selected from the group consisting of proteins, lipids (fats), sugars, nucleic acids, antibodies, cells, cellular structures, cellular components, and combinations thereof.

400. The coordination compound of claim 399, wherein said protein is selected from the group consisting of enzymes, growth factors, hormones, cytokines, and combinations thereof.

401. The coordination compound of claim 399, wherein said lipid (fat) is selected from the group consisting of phospholipids, glycolipids, steroids, and combinations thereof.

402. The coordination compound of claim 399, wherein said sugar is selected from the group consisting of heparin, chondritin, glycogen, and combinations thereof.

403. The coordination compound of claim 399, wherein said nucleic acid is selected from the group consisting of deoxoribonucleic acid (DNA), ribonucleic acid (RNA), peptide nucleic acid (PNA), and combinations thereof.

404. The coordination compound of claim 399, wherein said antibody is selected from the group consisting of polyclonal antibodies, monoclonal antibodies, Fab fragments, and combinations thereof.

405. The coordination compound of claim 387, wherein said linker is selected from the group consisting of peptides, lipids, and sugars.

406. The coordination compound of claim 387, wherein said linker is a substrate to, and is cleavable by, at least one type of an enzyme whose activity is induced or expressed during onset of a cardiovascular type of medical condition of a subject.

407. The coordination compound of claim 405, wherein said peptide type of said linker is a substrate to, and is cleavable by, a matrix metalloproteinase protease type of enzyme whose activity is induced or expressed during onset of a cardiovascular type of medical condition of a subject.

408. The coordination compound of claim 405, wherein said peptide type of said linker is a matrix metalloproteinase substrate selected from the group consisting of (1) a substrate of MMP-9, (2) a substrate of MMP-2, (3) a substrate of MMP-3, (4) a substrate of MMP-14, and (5) a substrate of MMP-1.

409. The coordination compound of claim 405, wherein said peptide type of said linker is a substrate to, and is cleavable by, a type of peptidase selected from the group consisting of serine-type peptidases, threonine-type peptidases, aspartic-type peptidases, and cystein-type peptidases.

410. The coordination compound of claim 405, wherein said lipid type of said linker is selected from the group consisting of glutaric acid, adipic acid, pimelic acid, suberic acid, azelaic acid, sebacic acid, roccellic acid, 5-aminopentanoic acid, 11-aminodecanoic acid, 4-aminophenylacetic acid, 4-(aminomethyl)benzoic acid, 7-aminoheptanoic acid, 6-aminohexanoic acid, and 4-aminobutyric acid.

411. The coordination compound of claim 405, wherein said sugar type of said linker is a substrate to, and is cleaved by, a type of sugar degrading enzyme selected from the group consisting of heparinase and hyaloronidase.

412. The coordination compound of claim 405, wherein said sugar type of said linker is selected from the group consisting of polysaccharide glycosaminoglycans, chlondroitin sulfate, dermatan sulfate, heparan sulfate, heparin, and keratan sulfate.

413. The coordination compound of claim 387, wherein said linker is a biocompatible synthetic polymer that is a substrate to, and is cleavable by, at least one type of a chemical whose activity is induced or expressed during onset of a cardiovascular type of medical condition of a subject.

414. The coordination compound of claim 413, wherein said biocompatible synthetic polymer is selected from the group consisting of synthetic polyethylene glycols, wherein a said synthetic polyethylene glycol is selected from the group consisting of polyethylene glycol 400, polyethylene glycol 200, polyethylene glycol-distearoylphosphatidylethanolamine, polyethylene glycol-caprolactone/trimethylenecarbonate copolymers, polyethylene glycol-(poly-lactic acid), S-nitrosylated polyethylene glycol, methoxy-polyethylene glycol, and dimyristoylphosphatidylethanolamine-N-[methoxy(polyethylene glycol)].

415. The coordination compound of claim 387, wherein said linker is a biocompatible synthetic bi-functional cross-linker that is a substrate to, and is cleavable by, at least one type of a chemical whose activity is induced or expressed during onset of a cardiovascular type of medical condition of a subject.

416. The coordination compound of claim 415, wherein said biocompatible synthetic bi-functional cross-linker is selected from the group consisting of synthetic m-maleimido-N-hydroxysuccinimide, bis[beta-(4-azidosalicylamido)ethyl]disulfide, bis-maleimidohexane, and sulfosuccinimidyl-[perfluoroazidobenzamido]-ethyl-1,3-dinitropropionate.

417. A medical device comprising a medical implant component having a metal surface (M) to which is chelated a chelator (C).

418. The medical device of claim 417, wherein said medical implant component corresponds to at least a section of at least a part having said metal surface of a whole medical implant.

419. The medical device of claim 418, wherein said medical implant is selected from the group consisting of a stent, a prosthesis, a catheter, a balloon, a shunt, a valve, a pacemaker, a pulse generator, a cardiac defibrillator, a spinal stimulator, a brain stimulator, a sacral nerve stimulator, an inducer, a sensor, a seed, an anti-adhesion sheet, a plate, a joint, a fin, a screw, a spike, a wire, a filament, a thread, an anchor, and a bone fixation element.

420. The medical device of claim 418, wherein said medical implant is a stent and said part is selected from the group consisting of a wire, a filament, a thread, of said stent; a film, a plating, and a coating, deposited upon at least a section of another part of said stent.

421. The medical device of claim 418, wherein said medical implant is a prosthesis and said part is selected from the group consisting of a plate, a joint, a fin, a screw, a spike, a wire, a filament, a thread, an anchor, another bone fixation element, of said prosthesis; a film, a plating, and a coating, deposited upon at least a section of another part of said prosthesis.

422. The medical device of claim 417, wherein said metal surface corresponds to an external side or/and an internal side of said medical implant component.

423. The medical device of claim 417, wherein said metal surface (M) there is a sub-population of exposed surface metal ions and atoms each being charged, uncharged, or polarized, and each being chelated to at least one chelator molecule of said chelator (C) in a form of a said metal surface (M)—said chelator (C) chelate type of coordination compound configuration.

424. The medical device of claim 417, wherein each chelator molecule of said chelator (C) has a negative charge, a zero charge, or a positive charge.

425. The medical device of claim 417, wherein each said metal surface (M)—said chelator (C) chelate type of coordination compound configuration formed between at least one surface metal ion or atom of said metal surface (M) and at least one chelator molecule of said chelator (C) has a total zero, positive, or negative, net charge.

426. The medical device of claim 417, wherein coordination number of each chelated surface metal ion or atom of said metal surface (M) is in a range of between two and twelve.

427. The medical device of claim 417, wherein mass and molar quantities of said chelator (C) bound on said metal surface (M) in a form of a surface coating are greater than 100 picograms and greater than 1 picomole, respectively, per square centimeter of said metal surface (M).

428. The medical device of claim 417, wherein metal chelated chelator molecules of said chelator (C) have a bonding potential or affinity and capacity for selectively binding, via chelating, free metal ions originating from a free metal ion source (W).

429. The medical device of claim 428, wherein said free metal ion source (W) is blood circulating through a cavity of a blood vessel.

430. The medical device of claim 417, wherein metal chelated chelator molecules of said chelator (C) have a bonding potential or affinity and capacity for selectively binding, via chelating, free metal ions originating from a free metal ion source (W), for forming an (M)-(C)-(W) chelate type of coordination compound configuration.

431. The medical device of claim 430, wherein said free metal ion source (W) is blood circulating through a cavity of a blood vessel.

432. The medical device of claim 430, wherein a said formed (M)-(C)-(W) chelate type of coordination compound configuration is firstly characterized by having at least two coordinate covalent bonds between a chelated surface metal ion or atom of said metal surface (M) and at least two coordinating groups of a said metal chelated chelator molecule of said chelator (C), and is secondly characterized by having at least two coordinate covalent bonds between at least two coordinating groups of said metal chelated chelator molecule of said chelator (C) and a chelated metal ion or atom previously being said free metal ion from said free metal ion source (W).

433. The medical device of claim 432, wherein mass and molar quantities of said chelator (C) or/and of said chelated metal ion or atom from said free metal ion source (W) in said (M)-(C)-(W) configuration bound on said metal surface (M) in a form of a surface coating are greater than 100 picograms and greater than 1 picomole, respectively, per square centimeter of said metal surface (M).

434. The medical device of claim 417, wherein said metal surface (M) is composed of a material selected from the group consisting of a metallic material, a semi-metallic material, and a combination thereof.

435. The medical device of claim 434, wherein said material includes at least one metal element, at least one metal alloy each of at least two metal elements, or a combination thereof.

436. The medical device of claim 435, wherein said at least one metal element is selected from the group consisting of nickel [Ni], titanium [Ti], platinum [Pt], iridium [Ir], tantalum [Ta], iron [Fe], cobalt [Co], molybdenum [Mo], chromium [Cr], beryllium [Be], copper [Cu], tungsten [W], vanadium [V], niobium [Nb], palladium [Pd], gold [Au], silver [Ag], zinc [Zn], aluminum [Al], iron [Fe], and a combination thereof.

437. The medical device of claim 435, wherein said at least one metal alloy is selected from the group consisting of a shape memory alloy, a stainless steel alloy, a nickel-titanium [Ni—Ti] alloy, a cobalt-molybdenum-chromium [Co—Mo—Cr] alloy, a beryllium-copper [Be—Cu] alloy, a cobalt-chromium [Co—Cr] alloy, a cobalt-tungsten [Co—W] alloy, a cobalt-chromium-tungsten [Co—Cr—W] alloy, a nickel-titanium-vanadium [Ni—Ti—V] alloy, a platinum-iridium [Pt—Ir] alloy, a copper-zinc-aluminum [Cu—Zn—Al] alloy, a platinum-tungsten [Pt—W] alloy, a cobalt-chromium-nickel [Co—Cr—Ni] alloy, a nickel-cobalt-chromium-molybdenum [Ni—Co—Cr—Mo] alloy, a titanium-aluminum-vanadium [Ti—Al—V] alloy, and a titanium-aluminum-nickel [Ti—Al—Ni] alloy.

438. The medical device of claim 417, wherein compounds of said chelator (C) are selected from the group consisting of bifunctional acids, amino acids, peptides, proteins, ethylenediamine, propylenediamine, diethylenetriamine, triethylenetetraamine, ethylenediaminetetraaceto, hydroxyquinolates, hydroxyquinones, aminoquinones, phenanthroline, acetylacetone, oxalic acid; 4,5-dihydroxy-naphthalene disulfonic acid; N-nitrosophenylhydroxyamine ammonium salt; diantipyrylmethane; 8-hydroxyquinoline; 5-amino-8-hydroxyquinoline; 2′,4′,5,7-tetrahydroxy-3,4-di-flavone; 3,5-pyrocatecholdisulfonic acid; nitrilotriacetic acid (NTA); diethylenetriamine-penta-acetic acid (DTPA); quinoline-2-carboxylate; histidine (amino acid); 6His (6 histidine peptide); N-acetylcystein amide (amino acid); D-penicillamine; RGD (peptide); Cu/Zn superoxide dismutase (protein); Atoxl (protein); hemoplexin (protein); 2,3-dimercapto-1-propansulfonic acid (DMPS); mecaptosuccinic acid (DMSA); S-cystaminyl-EDTA; amino tris methylenephosphoric acid (ATMA); 1-hydroxyethylidene-1-bisphosphonate (HEBP), and combinations thereof.

439. The medical device of claim 417, wherein said chelator (C) is used for preventing or/and treating a cardiovascular type of medical condition of a subject.

440. The medical device of claim 439, wherein said medical condition of said subject is selected from the group consisting of restenosis, in-stent restenosis, thrombosis, and a combination thereof.
